(19) United States
(12) Patent Application Publication
(10) Pub. No.: US 2026/0273081 A1
(43) Pub. Date: Sep. 17, 2026

(54) INHIBITORS OF ETS2-ACTIVATING MOLECULES FOR THE TREATMENT OF INFLAMMATORY DISEASES

(71) Applicant: The Francis Crick Institute Limited, London (GB)

(72) Inventors: James Christopher Lee, London (GB); Christophe Jean-Marie Bourges, London (GB); Christina Therese Stankey, London (GB)

(21) Appl. No.: 19/471,756

(22) PCT Filed: Mar. 27, 2024
(86) PCT No.: PCT/EP2024/058343

§ 371 Date: Oct. 2, 2025

(30) Foreign Application Priority Data
Apr. 2, 2023 (GB) 2304900.0
May 5, 2023 (GB) 2306675.6

(51) Int. Cl. A61K 47/68; A61K 31/4184; A61K 31/4523; A61K 31/519; C07K 16/28; C12N 9/22; C12N 15/11; C12Q 1/34; C12Q 1/6825; C12Q 1/6883; G01N 33/50

(52) U.S. Cl. CPC A61K 47/6803; A61K 31/4184; A61K 31/4523; A61K 31/519; A61K 47/6849; A61K 47/6889; C07K 16/2896; C12N 9/226; C12N 15/111; C12Q 1/34; C12Q 1/6825; C12Q 1/6883; G01N 33/5023; G01N 33/5055; C12N 2310/20; C12Q 2600/142; G01N 2333/4703; G01N 2333/922; G01N 2500/10

(57) ABSTRACT

The present invention provides inhibitors of proteins that activate Erythroblast Transformation Specific proto-oncogene (2) (ETS2), and their use in treating or preventing inflammatory and/or autoimmune diseases. The inhibitors are generally MEK inhibitors, HSP90 inhibitors, RAF inhibitors, SRC inhibitors, and/or ERK inhibitors. The invention also encompasses methods of diagnosis and methods of screening for agents that reduce macrophage activation.

### FIELD OF THE INVENTION

The present invention relates to the treatment and/or prevention of inflammatory and/or autoimmune diseases or disorders with Erythroblast Transformation Specific proto-oncogene 2 (ETS2) inhibitors and/or inhibitors of an enhancer region at chr21q22. The invention also encompasses methods of diagnosis and methods of screening for agents that reduce macrophage activation.

### BACKGROUND

The global incidence of autoimmunity is steadily rising with nearly 5% of the world's population affected by one or more autoimmune/inflammatory disease. These heterogeneous conditions, which range from inflammatory bowel disease (IBD) to systemic lupus erythematosus and multiple sclerosis, share an urgent need for better treatments, but only ~10% of drugs entering clinical development ever become approved therapies [1]. This high failure rate is principally due to a lack of efficacy [2]—exposing an increasingly urgent need to better understand disease mechanisms. Genetics provides a unique opportunity to do this, with hundreds of regions of the human genome now directly linked to the pathogenesis of at least one autoimmune disease [3]. Indeed, drugs targeting pathways identified by genetics are much more likely to constitute effective therapies [4,5].

To fully realise the potential of genetics, however, knowledge of where disease risk variants lie must first be translated into an understanding of how they contribute to disease [3]. This is a formidable challenge since most disease-associated genetic variants do not lie in coding DNA, where effects on protein sequence/structure can be easily determined, but rather in the enigmatic non-coding genome where the same DNA sequence can have different biological consequences depending on the cell-type and/or external stimuli [3]. Most risk variants are thought to affect gene regulation [6], but the need to identify the causal gene(s)—which may lie up to one million bases away- and the causal cell-type(s), which may only express the causal gene under specific conditions, have so far hindered attempts to discover disease mechanisms. For example, although over 240 risk loci have been reported in genome-wide association studies (GWAS) for IBD7, fewer than 10 have been mechanistically resolved and, to date, none have led to new therapies.

### SUMMARY OF THE INVENTION

Here, the inventors demonstrate that an intergenic “gene desert” on chr21q22, which predisposes to several chronic inflammatory diseases, is a monocyte/macrophage-specific distal enhancer of ETS2 and delineate the gain-of-function molecular mechanism by which the risk haplotype alters ETS2 expression. Moreover, by combining CRISPR-based loss-of-function approaches in primary human macrophages cultured under chronic inflammatory conditions-targeting both the intergenic region and ETS2—with methods to deliver dose-dependent overexpression, we show that ETS2, a gene mostly studied for its role in cancer, is a central controller of macrophage inflammatory responses.

Consistent with this, the inventors have identified that the transcriptional footprint of ETS2 is detectable in the diseased tissues of multiple inflammatory conditions and is more enriched for IBD risk variants than most previously described disease processes, including autophagy. By screening a database of over 30,000 cellular signatures, the inventors have discovered potential means of therapeutically targeting this pathway and validate one class of approved small molecules using both in vitro and in vivo models—thereby illustrating the potential of genetics to uncover novel disease biology.

The present invention provides a method of treating or preventing a disease in a subject, the method comprising administering an inhibitor of a protein that activates Erythroblast Transformation Specific proto-oncogene 2 (ETS2) to the subject, wherein the protein that activates Erythroblast Transformation Specific proto-oncogene 2 (ETS2) is selected from the list consisting of: MEK, HSP90, RAF, SRC and ERK, and wherein the disease is an inflammatory and/or autoimmune disease. The present invention provides a method of treating or preventing a disease in a subject, the method comprising administering an Erythroblast Transformation Specific proto-oncogene 2 (ETS2) inhibitor to the subject. The present invention provides a method of treating or preventing a disease in a subject, the method comprising administering an inhibitor of the chr21q22 enhancer (SEQ ID NO:1) to the subject. The present invention provides a method of treating or preventing a disease in a subject, the method comprising modifying or deleting a portion of the chr21q22 enhancer (SEQ ID NO:1) to the subject, optionally wherein the entire chr21q22 enhancer (SEQ ID NO:1) is deleted. The present invention provides a method of reducing macrophage activation by contacting said macrophage with an inhibitor of Erythroblast Transformation Specific proto-oncogene 2 (ETS2). The present invention provides a method of reducing macrophage activation by contacting said macrophage with an inhibitor of the chr21q22 enhancer (SEQ ID NO:1). The present invention provides a method of reducing macrophage activation by modifying or deleting a portion of the chr21q22 enhancer (SEQ ID NO:1), optionally wherein the entire chr21q22 enhancer (SEQ ID NO:1) is deleted. The present invention provides a method of reducing proinflammatory cytokine production by contacting a macrophage with an inhibitor of Erythroblast Transformation Specific proto-oncogene 2 (ETS2). The present invention provides a method of reducing proinflammatory cytokine production by contacting a macrophage with an inhibitor of the chr21q22 enhancer (SEQ ID NO:1). The present invention provides a method of reducing proinflammatory cytokine production by modifying or deleting a portion of the chr21q22 enhancer (SEQ ID NO:1), optionally wherein the entire chr21q22 enhancer (SEQ ID NO:1) is deleted. The present invention provides a method of reducing reactive oxygen species (ROS) production by contacting a macrophage with an inhibitor of Erythroblast Transformation Specific proto-oncogene 2 (ETS2). The present invention provides a method of reducing reactive oxygen species (ROS) production by contacting a macrophage with an inhibitor of the chr21q22 enhancer (SEQ ID NO:1). The present invention provides a method of reducing reactive oxygen species (ROS) production by modifying or deleting a portion of the chr21q22 enhancer (SEQ ID NO:1), optionally wherein the entire chr21q22 enhancer (SEQ ID NO:1) is deleted. The present invention provides a method of reducing macrophage phagocytosis by contacting said macrophage with an inhibitor of Erythroblast Transformation Specific proto-oncogene 2 (ETS2).

The present invention provides a method of reducing macrophage phagocytosis by contacting said macrophage with an inhibitor of the chr21q22 enhancer (SEQ ID NO:1). The present invention provides a method of reducing macrophage phagocytosis by modifying or deleting a portion of the chr21q22 enhancer (SEQ ID NO:1), optionally wherein the entire chr21q22 enhancer (SEQ ID NO: 1) is deleted. The present invention provides a method of reducing macrophage migration by contacting said macrophage with an inhibitor of Erythroblast Transformation Specific proto-oncogene 2 (ETS2). The present invention provides a method of reducing macrophage migration by contacting said macrophage with an inhibitor of the chr21q22 enhancer (SEQ ID NO: 1). The present invention provides a method of reducing macrophage migration by modifying or deleting a portion of the chr21q22 enhancer (SEQ ID NO: 1), optionally wherein the entire chr21q22 enhancer (SEQ ID NO:1) is deleted.

In some embodiments, a reduction in macrophage activation is indicated by one or more of a reduction in cellular activation, a reduction in proinflammatory cytokine production, metabolic reprogramming, a reduction in reactive oxygen species production, and/or a reduction in macrophage migration. In some embodiments, a reduction in proinflammatory cytokine production comprises a reduction in the level of one or more of TNFα, IL-1, IL-6, IL-8, IL-12, IL-23 and/or IL-18. In some embodiments, a reduction in reactive oxygen species (ROS) production comprises a reduction in the level of one or more of hydrogen peroxide, hydroxyl radical, superoxide anion and/or singlet oxygen. In some embodiments, a reduction in macrophage phagocytosis is indicated by a reduction in uptake of particulate matter (for example bacteria) by macrophages, quantified by an in vitro or in vivo assay. In some embodiments, a reduction in macrophage migration is indicated by and reduction in in vitro measures of macrophage motility (for example quantified by a chemotaxis assay) and/or a reduction in the total number of macrophages that accumulate at sites of inflammation in vivo.

The present invention provides a method of treating a disease in a subject in need thereof, the method comprising administering to the subject an Erythroblast Transformation Specific proto-oncogene 2 (ETS2) inhibitor, thereby reducing macrophage activation in the subject. The present invention provides a method of treating a disease in a subject in need thereof, the method comprising administering to the subject a chr21q22 enhancer (SEQ ID NO:1) inhibitor, thereby reducing macrophage activation in the subject. The present invention provides a method of treating a disease in a subject in need thereof, the method comprising modifying or deleting a portion of the chr21q22 enhancer (SEQ ID NO:1), thereby reducing macrophage activation in the subject.

In some embodiments, the subject has an inflammatory disease and/or autoimmune disease. In some embodiments, the inflammatory disease is selected from the list consisting of: Crohn's disease, ulcerative colitis, inflammatory bowel disease (Crohn's disease and/or ulcerative colitis), ankylosing spondylitis, primary sclerosing cholangitis, Takayasu arteritis and rheumatoid arthritis. In some embodiments, the disease is an autoinflammatory disease. In some embodiments, the disease is a chr21q22-associated disease. In some embodiments, the disease is selected from the group consisting of Crohn's disease, ulcerative colitis, primary sclerosing cholangitis, ankylosing spondylitis, and Takayasu arteritis.

The present invention provides an inhibitor of a protein that activates Erythroblast Transformation Specific proto-oncogene 2 (ETS2) for use in a method of treating or preventing a disease in a subject, the method comprising administering an inhibitor of a protein that activates Erythroblast Transformation Specific proto-oncogene 2 (ETS2) to the subject, wherein the protein that activates Erythroblast Transformation Specific proto-oncogene 2 (ETS2) is selected from the list consisting of: MEK, HSP90, RAF, SRC and ERK, and wherein the disease is an inflammatory and/or autoimmune disease. The present invention provides an Erythroblast Transformation Specific proto-oncogene 2 (ETS2) inhibitor for use in a method of treating or preventing a disease in a subject. The present invention provides an inhibitor of the chr21q22 enhancer (SEQ ID NO: 1) for use in a method of treating or preventing a disease in a subject. The present invention provides an Erythroblast Transformation Specific proto-oncogene 2 (ETS2) inhibitor for use in a method of reducing macrophage activation by contacting said macrophage with an inhibitor of Erythroblast Transformation Specific proto-oncogene 2 (ETS2). The present invention provides an inhibitor of the chr21q22 enhancer (SEQ ID NO:1) for use in a method of reducing macrophage activation by contacting said macrophage with an inhibitor of the chr21q22 enhancer (SEQ ID NO:1). The present invention provides an Erythroblast Transformation Specific proto-oncogene 2 (ETS2) inhibitor for use in a method of reducing proinflammatory cytokine production by contacting a macrophage with an inhibitor of Erythroblast Transformation Specific proto-oncogene 2 (ETS2). The present invention provides an inhibitor of the chr21q22 enhancer (SEQ ID NO: 1) for use in a method of reducing proinflammatory cytokine production by contacting a macrophage with an inhibitor of the chr21q22 enhancer (SEQ ID NO:1). The present invention provides an Erythroblast Transformation Specific proto-oncogene 2 (ETS2) inhibitor for use in a method of reducing reactive oxygen species (ROS) production by contacting a macrophage with an inhibitor of Erythroblast Transformation Specific proto-oncogene 2 (ETS2). The present invention provides an inhibitor of the chr21q22 enhancer (SEQ ID NO:1) for use in a method of reducing reactive oxygen species (ROS) production by contacting a macrophage with an inhibitor of the chr21q22 enhancer (SEQ ID NO:1). The present invention provides an Erythroblast Transformation Specific proto-oncogene 2 (ETS2) inhibitor for use in a method of reducing macrophage phagocytosis by contacting said macrophage with an inhibitor of Erythroblast Transformation Specific proto-oncogene 2 (ETS2). The present invention provides an inhibitor of the chr21q22 enhancer (SEQ ID NO:1) for use in a method of reducing macrophage phagocytosis by contacting said macrophage with an inhibitor of the chr21q22 enhancer (SEQ ID NO:1). The present invention provides an Erythroblast Transformation Specific proto-oncogene 2 (ETS2) inhibitor for use in a method of reducing macrophage migration by contacting said macrophage with an inhibitor of Erythroblast Transformation Specific proto-oncogene 2 (ETS2). The present invention provides an inhibitor of the chr21q22 enhancer (SEQ ID NO: 1) for use in a method of reducing macrophage migration by contacting said macrophage with an inhibitor of the chr21q22 enhancer (SEQ ID NO: 1).

In some embodiments, a reduction in macrophage activation is indicated by one or more of a reduction in cellular activation, a reduction in proinflammatory cytokine production, metabolic reprogramming, a reduction in reactive oxygen species production, and/or a reduction in macrophage migration. In some embodiments, a reduction in proinflammatory cytokine production comprises a reduction in the level of one or more of TNFα, IL-1, IL-6, IL-8, IL-12, IL-23 and/or IL-18. In some embodiments, a reduction in reactive oxygen species (ROS) production comprises a reduction in the level of one or more of hydrogen peroxide, hydroxyl radical, superoxide anion and/or singlet oxygen. In some embodiments, a reduction in macrophage phagocytosis is indicated by a reduction in uptake of particulate matter (for example bacteria) by macrophages, quantified by an in vitro or in vivo assay. In some embodiments, a reduction in macrophage migration is indicated by a reduction in in vitro measures of macrophage motility (for example quantified by a chemotaxis assay) and/or a reduction in the total number of macrophages that accumulate at sites of inflammation in vivo.

The present invention provides an Erythroblast Transformation Specific proto-oncogene 2 (ETS2) inhibitor for use in a method of treating a disease in a subject in need thereof, the method comprising administering to the subject an Erythroblast Transformation Specific proto-oncogene 2 (ETS2) inhibitor, thereby reducing macrophage activation in the subject. The present invention provides an inhibitor of the chr21q22 enhancer (SEQ ID NO:1) for use in a method of treating a disease in a subject in need thereof, the method comprising administering to the subject a chr21q22 enhancer (SEQ ID NO: 1) inhibitor, thereby reducing macrophage activation in the subject.

In some embodiments, the subject has an inflammatory disease and/or autoimmune disease. In some embodiments, the inflammatory disease is selected from the list consisting of: Crohn's disease, ulcerative colitis, inflammatory bowel disease (Crohn's disease and/or ulcerative colitis), ankylosing spondylitis, primary sclerosing cholangitis, Takayasu arteritis and rheumatoid arthritis. In some embodiments, the disease is an autoinflammatory disease. In some embodiments, the disease is a chr21q22-associated disease. In some embodiments, the disease is selected from the group consisting of Crohn's disease, ulcerative colitis, primary sclerosing cholangitis, ankylosing spondylitis, and Takayasu arteritis.

In some embodiments, the ETS2 inhibitor or chr21q22 enhancer inhibitor comprises an antisense nucleic acid molecule, a small interfering RNA (siRNA), or a short hairpin RNA (shRNA). In some embodiments, the ETS2 inhibitor or chr21q22 enhancer inhibitor comprises a small molecule, inhibitory peptide, an antibody or a nanobody. In some embodiments, the antibody is a monoclonal antibody that targets surface markers specific to ETS2-positive macrophages to inhibit effector function and/or induce destruction. In some embodiments, the nanobody targets surface markers specific to ETS2-positive macrophages to inhibit effector function and/or induce destruction.

In some embodiments, the ETS2 inhibitor or chr21q22 enhancer inhibitor comprises a proteolysis-targeting chimera (PROTAC) that targets ETS2 for degradation, a molecular glue that targets ETS2 for degradation, an antibody-drug conjugate, an antibody-RNA conjugate, an esterase-sensitive motif-conjugated drug or a nanobody-drug conjugate. In some embodiments, the antibody-drug conjugate comprises an anti-CD163 antigen binding molecule, optionally wherein the antigen binding molecule is an antibody. In some embodiments, the antibody-drug conjugate comprises a MEK inhibitor, optionally wherein the MEK inhibitor is selected from the list consisting of Selumetinib, Trametinib and Cobimetinib, preferably wherein the MEK inhibitor is Selumetinib.

In some embodiments, the antibody-drug conjugate comprises a cleavable linker, optionally wherein the cleavable linker is glucuronide-based (e.g. GlyPro). In some embodiments, the antibody-drug conjugate comprises a non-cleavable linker, optionally wherein the non-cleavable linker is maleimide-PEG3-based.

In some embodiments, the esterase-sensitive motif-conjugated drug comprises cyclopentyl L-leucinate or cyclopentyl(S)-2-amino-2-cyclohexylacetate. In some embodiments, the esterase-sensitive motif-conjugated drug comprises a MEK inhibitor, optionally wherein the MEK inhibitor is selected from the list consisting of Selumetinib, Trametinib and Cobimetinib. In some embodiments, the esterase-sensitive motif-conjugated drug comprises a linker. In some embodiments, the linker is one or two carbons in length.

In some embodiments, the ETS2 inhibitor comprises a CRISPR nuclease system comprising a CRISPR-associated protein (Cas protein) and a guide RNA (gRNA) that specifically binds to an ETS2 genomic nucleic acid sequence, optionally wherein the ETS2 genomic nucleic acid sequence comprises the sequence of SEQ ID NO:5. In some embodiments, the chr21q22 enhancer inhibitor comprises a CRISPR nuclease system comprising a CRISPR-associated protein (Cas protein) and a guide RNA (gRNA) that specifically binds to the chr21q22 enhancer nucleic acid sequence, optionally wherein the chr21q22 enhancer nucleic acid sequence comprises the sequence of SEQ ID NO:1.

SEQ ID NO: 5 (chr21: 40176507-40197479) is shown below.
(SEQ ID NO: 5)
GTGATTGTTTCAGCCGTATAGATTCTGACTGTGACTCATGCCACGGTGATACCCGGAGACCTTC
GGGGACCCAGAAACATTAAGGTGCACCGATATCTCAATTATCCGGATAACTCTAGACCTAGAGT
CACCATGGAAACCTAGAACCCTGAGTCACAGAGAAGCTGGGCCATTCCTCTCCCTTCCCGGTGG
TGCCGCAGTTCTTGAGATGTGAGCAGCTTTCAGCCCCGTGGCCTCGCCTAAGCTGGGGTTTCCT
ATTTCATCCACAACTTCAGGAATCTAAACTTTCCTGCAAAATATTGAGCATGTGACCTGAGTCC
AAACAGCCATGGACTCAAGCACCTTCTGATAAAAACGGAAACCACAGTGTAGGGGGTGGTTTTG
ACCAGGGTTTCGGAGTCAGATAAACTCAGGTTTCCACCTTGAATTTCTCATCAATCTGACGTGG
CAGGTTATTCATCCTAAGGTTCAGTTCCCACCTGTGTAAAGTGGGAGCCGCAAGTCCTCCGAGA
GTGACGATGATGTGCGTGGAGTGCCCAGCCCAGATTGAAGCGCGGCCAAGGCGGGTCGCTATCT
GGGCACCGCTCAGCTCCAGAGGGCGCCACTCCCGCGGAGCCTGCGGGATCGGGGCTTCCCGGGA
GCAGCGCGATCAGCACCACGACTCGGGGACACAGCCAGGGCCCGGTTTCTACAGGAAGCGCCTC
ATTTGGAGCCTTTTTGTGATAGAATGATCATTAGTCCTAAGCCCATTCAGAGGTTCAAGAATGG
GGTCGGCTCAATTTCAGGGCCTTATTACCCAAGCCCGGCTGCCCTTCGGTGCCACCAGCACCAC
TGCTCCGTCGCTGCGGAATTCCAAAGGCAGGTTTGGCGTTAGGGCCTTGGCCCCAGAGAGGACG
CCGAGCGCTCCACGGAAAGTCTCCGCCCGGCTCCCAGGGCGCACACTCGCGCGCACGTGGGGCC
GAGGCCCTGCTCCCGGGGCCTCAGGGCCAGCCGGCGAGGGACCCAGCCGAGTGACAGCAGGAGG
CGGAGGGAAGGTTGGGCCGGAAGGTGTCAGCCCCGCCCCGCGCTCCCTCGCCGCCTCCGCCCTC
CTCTTCTCTCCCCTCGTGCGTTCCCTCTCCTCTCCCTCCGCTCCCCCAACCCTCCTGCTGCCCC
CTTCCCTCTCCTCCCGCTTCTCCCCATCCTGCCTACCTCCCTTCCCCTCCTCTTCTCTCTCCTC
CCCTTCCCTCCCCTCTCTCTTCTCTCCTCCCTCGTTTCCTCCCCTCCCCTCCACTCGGCCGTCC
CTCCTTCCTCCTCCCTCCTCCCTCCTCCTCCCGCTCCTGAAGAGCGCGCCGCGTGGGGGACGGC
CCGGTTACTTCCTCCAGAGACTGACGAGTGCGGTGTCGCTCCAGCTCAGAGCTCCCGGAGCCGC
CCGGCCAGCGTCCGGCCTCCCTGATCGTCTCTGGCCGGCGCCCTCGCCCTCGCCCGGCGCGCAC
CGAGCAGCCGCGGGCGCCGAGCAGCCACCGTCCCGACCAAGCGCCGGCCCTGCCCGCAGCGGCA
GGGTAAGAGCTGGGCCCGCAGAGAGCGCCCGGCGCGCGGCTCCAGTCCCATGGAGGGTCACCCG
GGGCCTGGGGGGGGGTCGCGGGGGGCACTGACACGCAGATCTCGGGGCGCTGCCGGGGGTGCAG
GTGGGGGTGGCGGCTGCTGCGAGGACTCTAGGGGCGCGCGTCTGAGTTCCGCGCCGGCTCGTTT
TCCGGTTATGGAGTGGCCTCCGGGGCTGGCGGGGTCGGCCGGGGGGTTCCTGCGTGCTAGGGCC
GCTGTCTTCGGGGTCGCCTAGCGGCGGGCGCGGCCAGGGCGCGCTGGCTTGTTTCGCTCGCTTT
TGTTTTTAAAAGGAAACGCAGGCCTGGTAGGGGGTCCTGCCCAGTGGATGTCCCGGCGAACATG
ATTTCGCGAACGGGAGTGGGGGCACAGGAGAGCGTGTCCGAGGTGGCCTGGCGCCCCGGCTTTG
AGGGTGACTTCCTGGAGCGGCGCCGGGCCCGGAGGATCTGGGGCGCCCAAGACACCTGAAGGCT
GCGGCACCGCGGGAACCTGCGGGGCGCGGGGTGCCATGGTCACCTGCTCGCCGCGTCCAGGGCC
CGGGCTGGGGACCCCTCGGTCGTGCGAGGAGAGCGTGGGGAACCTGTCGGAAATGAGATCTGGT
TGCGCTGGGCTGCCTTTATTTTCTCGTTCCTACAGCATTTGAATGAAGAGGTAACTGAGTGTTT
GCTTGTGTGTGTTTGGGTTGCGTGTGTGTTATCCTATTTTATTTTTTACGGCAGGAGACCTTTT
ATGTTAGCCTGTACACAATTTCAGGGTAGCCTAAAACAGTAGTTGACGCGCTAGTTATTTAGAA
AGTAAAGAAATGAGCAGTCCCCTTTGGAGACAGGAGTAATTTATTTTAATTCGCCAGTAAGAGA
TTATATTTGTTCCATACAATGGAAGCGCCTGTGCGTTATCTGCCTGCCCACACCACCACCAATC
AGAGAGATAAATCTCCACGTTAAGCTTTATTAAAATTCTGAGTAGTAGCGCAGCACAGTAAATG
TTAGCACCGACCATCCGCAGGGAAATGCTCAGATACTCATTTCTGGGATTAGTCATTCATCTCT
CTAAACTGATCGCTTTCAAAGCGAAAAAAGCAAACTACCTTCAAATGTGTGTTACAAGGTTGTG
TTGTTTCTTTTTTTTGTTTGTTTTCAAATTTATCTAAATAATCTTACATAAGTGATTACTTGTT
CCAATAAGGGTACTGTTGTCAGGCAAATTCTCCTTGTTTTTAAATGTAACCATTCTCAGCTGTT
ACATTTCTGCTCTTTATCCTTTTTTTTTTTTTTTTTTGGTTTAAAAGCAGCGATCCATCAGCAA
CACCAAACTTGAAATTGATTTATGTGGAAAAACTTGGCTTGTCTGCCATCTAACAAGCCCTGTT
GAGTAAAATAAGCAAGCTTAAATTTGATTAGTTGTGTGTCTGCCTGAAACCATTTAGTAGAGCA
CTTTAATTCTGCATGGTTTTTACAAAACTCATTAAAAGCTGGACAACAAAAGAATTCTATTCTG
TAGCTAGTAATTACAGCTCTTTATGTGGGAGTGGTAGGCTGCCTTTTCTCCTGGTATTTGTACA
CAAAAGCTGGGGAGAGCTTTTCCACTGCCTTCCACATTCATTCTCTGTCCATCTCAGAGAAGCT
GAAAAACAGAATCAAGTCAGCAAGCAGTCTGCAAGCCTAAGGAAGAAAAAGAGCTGACCCATCC
GGCTCTGGATGGATAACTGGCAACACCCAAGATGTCTATGATTTTGGCCTTCTGTTTGAGGCTT
GTTTATTTCTTTAATCAGCAGCTACCAGGACTTATTTCATGCAGAGAAATGACATAAAGCTCTA
GAGGAAGGAGCATTTCACATTGGAAGGAGTAGTGAGAAGGGCCTGATCTGATAATATCAGGATG
GGTGGGAAGGGTCCATTCCTCATCATTCATGTTCTCCCGGCACAGAGGACTGTGTGGAGGACTC
AGGATTTAGGACCATACTCAACTGGGGTTTGATCTCCACTTTGCCCTGCGGTGGCCCCTCTGGT
AGCTATGTGGATCCTGGGCAGATTGTCCATGTTGCTGGGCCTCCGTTGCTTCACTGTATGACGT
CTGCCTCTCAGGGATGCAGGAAGATTCAATTAGGTAGGAGTGGATGGTCCCCCAGCACGGGGCT
TAGCTCTTAGTAACTAGGCATTCTGGCAATGGCAGCCATCAATAGGAGAGACTATTGATTGTTG
AGTGAATATAAGTGGCATAATCCACCAAGTGGAGATTCTGAGTGAATGTGGCCACTTTGGACCC
AAGACTGCTGCCCCTCTCTGTCCTCTCATAGGACTATTATTTATTGCCAGTGCTGACCAGCCTT
TGTTGGCTACCATGATCATTACCTGCACTCCGAGTGTTTTAACAGAATTTTCTGATGCCTCTGG
CCTCACCTGCTGCCTTTTAGCCAAGAGGGGTGTGTGTGTATGTGTGTGTGTGTGTGTGTGTGTG
TGTTGGGTGTGTGTTGGGTAGCAGGGTTCTTGGAAGGTCTGGTTTAGCCATAATGTTGATCATA
AAGAGAAAACAAGAAAATAAATGCAAATTAAATAGTTCGGTGAGCCTTTGTTATTTCGCCACAT
CCAGTAAGTTCTATCAGTTGAATGTTGTCTGAATTACTTAAAAAGTTCTCAGTTGTAAACAGGT
AAGAGCACCCAGTGGAGGCCATGGAGCTAGGCTTAGAGCTTTGCCACCAGCCTTCAGTTCCATA
GCCATGAACCTGGGAGGGCAAGGAATCACCCTAGAGGTGAAGGCCATTCATCGGTGCTAACCCA
AGGTCTCTGAAAATGAGTTTTAGTTACTCTATTATCTAAGTATCTTTCTTACTGATATGTAACT
TACCTGGCTGAACAATTACATTTTAGAGCTCCAAATAAATTTTCAGCCTATTATGTCATTGTCC
AATTACAAGCCTCATTGTAATAGCCATGTTAAAAACAATAAAGCCGGTGAAATTGATTTCAGTG
TATTTCAAATTAACCTAATATATTCGAAATATTATGTATGTATAAAAATTATTGAGCTTTTTTC
TTTTTTCCACTAAGTCTTTGAGAGCTAGTGTGTATGTGTGGATTTGTACTAGCCACTGGTTGAG
AGCTCTGTAGCCACACATGGCAAGTGGGTACCATATTGCTTAGCCCAGGCCTAGTAGAAGATTA
AGACTCAGTCAGAGGAAGTAAGGCAGATCCGTGCTTGGAATCAGTTCCTCTTGAAGAAGTGCAC
AGCCAAGCAACACACAAAGCAGAAATATTTTGAGCTCTGTATTCAAAGCAGGAAGATCTGGGAC
CATGTAAGGGGGTTTGGTCTTCAGTTCTCCCTTTGATCCCTTCCCTGGGTGTGTGCTTTCTTAC
AGCCTGGAGAGTGGAAGGGAAGAGAGGGGGCTTCCTGGACTCCTTTCCTGTCTCTAGAAATGAC
CCCTGCAGCTGTCCTGAAGCTCTCAGGTTAAGCTGATTGTGTCCAGAGCAGACAGGAGGCGGCC
CACTGCCCCACTGTACCCACCCAAGCACTCACCTAAGCTGGCCATGGAAATGTGAATGAAAAGC
CTGACCCAGGCCCTCTTGCCCTGCTAACCCAGCAAGGAGGCTCTGACCAGCCAGCCAGCAAGTG
ACTTTTTCTGTGACATCTAAGTGGAATTCAGTGAGGAATCAGCATTATTATTCAATCACTGAAA
CAGCATATCCCATTGTGATACTGAGAAATGCTTTCCTCTTGAGACCTTTGGGAGGACAGCCATT
TAAACAAGAATTCCCTGCATTCACTTTTGAATTACCTGTTAGCTGTAGAACTCAGCCAGGATCA
AGTCTTGATGCCACCAATGATGAGTTTAGTGTACTTAATCTTTTGCCTCTTTGACTTTTTTTTT
TTCTTTTTTAAGATGAATGATTTCGGAATCAAGAATATGGACCAGGTAGCCCCTGTGGCTAACA
GTTACAGAGGGACACTCAAGGTGAGTGGGCAAGTCTTAATTTTTTTTTTTAATTGGAAAACTCG
ATCTCTAGGAGGAAAGAAAAAAAAGGCCTGGGTCCCAGAAAACTGGTTGTAGCCCTAGCTTCTT
AATTTATTGGCCATGTGACTATAGACTATTTGCTTACTTGCTCTGGTCCTCAGTTTTCTGGTCT
GCAGAATGGGTATCTTGATGTGTGTTGTTCTTCTCCTCCACGATATGAAACCAAAGGCCTTTAT
AGCTTGTTTTCTGAGGCTCCTTCCAGGTGAAAATCCTATAACTCCCAATAAAACAGCTGGAGAG
GCTGTCTCTACTGCCTTTTCTGTCTACCCAGGAGTTTCCTTGGTTGATGGGAAGCTGTTTAAGA
ACTGAAATGAGAGAGAAGAGGCAGACAGACCCAACCGCTACAGCTCCTATCAGAAAGGGCAGCG
CCCGTGGAATTGTAGGTAGCCTGTTACTGGCTCAGAGAACAAGCAGGCCTACTTATGTTAAACC
AAAAACCAGATGAAAAATGAAAGCGCCCTCTCTGCTGCATAAACATTTTATGTAGCTTCTATCA
TCATTCCTTTTGGGAACATTCTTTAGTAGCACAAACTGGTAATCTCCCACTTGTCTTCTCCAAA
TATGACAGCAAGATTTGATTGTTTTCAGTATTATGTAGTAAACATGTTTCAGAAGCATGATTTT
AAAATTGGCCTCCTCAAAGTTTAGCGTCTTGCATAATGATGATGTACGTCTCTGGCATATTACA
TTTTCCTTTGTATATCATTATTGAGGTTATTTGTCTGATATGACCCAAAGAGGCAAAACTCAGC
ACAGTCCTTTCTGCAGTATTCTAAAGGTCATCAAACTTCAGCCTAGTGAGTCTGCTTGTTTGAT
TTGGCCGGACATTTTAAGCATGGCAGAAGTGGTACAAGAAATCATGGTATTAAGTTGAAACCAC
ACCCCTTAGAAAAATCCTTCTATTAATTCAAATAATTTGACGATGCTTATGCGGTTTCTGAAAA
GAAGCAGTCGTTGCTGAAATTGATGTGTTGAAATAGGAAGCACAGATTTGTATTGTCTTTTGGC
TTCCTGGCTTTAAAAAAAAAAAAAAAGAATTTACGGTTAGGAAAGGCATTTCTTAGTCACCTGG
GCAATGCTTGTGGGAACTTTAAAAGTTTATTGGAGGTTTCACCAAAATTAGCTCTGCTCTAAAG
TAGTTTGTAAATCCAGATGAGTAATTTGCACTTGCCTGATTGATCGGTTTTCCTCGTAGATCTC
AGGCAGTTGGTCATCATTGACTTGGATTTTTTTGGGCTGGGCTCCAGCCTCTGCCTGCCCTCTT
TTGTAATGCGTCATTCATTGTTCTGCATTTCTAAAAAGAAAGCGGGTAACCGATTTTATTTGTA
AATGTGTTACACATTTTCTTATTGATGACTCAACAGATGGGTCATTGTATTACACATAACATGG
GATTTCCAGCTTATGTTGGAAAAATATAGTCTTTCTTCACGTGTTCCTCAACAATTTAGGCGAT
GATGAATCCAGATATTATCGGTTACTTTCAAATGAGTCTTACATTAAAAGGTATTTCTTTCATA
AATGTTATGTAAGATGCTCAGAAATCGAGTTAGTGTAAGGTTTTATCTTTCTGTGTGTCTCTTC
TTTTCTGCAGATAGTTTAGTTTAACTTTAAACCTATTTCCTTTGGAAATCTTTCCCCTTAGTAG
GTGATTTGGGTTTTTTTTGTTTTGTTTTATTTGTTTTTTCGTTTTTGGTTTTAAGCAACAGGGT
CCTTCTCTGTTGCCCAGGCGGGAGTACAGTGGCTTGATCGTAGTTCACCGCAGACTTGAACTCC
TGGGCTCAAGTGATCCTCTGGCCTCAACCTCATAAAGCACTGGGGTTACAGGCATAAGCCACCC
CGCCTGGCCTGGGGGCGGGGAGGGGGTTCATTTTTAAGACACTTTACTTGTTATAAAATGAAAC
GTTAAGGAATTTTGTCATGTATTACTATTTTCCAAACCATTGGAATTGATGTGGAAAAGCATCT
CTAGTTTCAGTTAAATTGTTTCATGGCAAACATTTCAAAATAAATGCTTCCAAAGATAAAGTAG
TGTATTTCATTGTTTGATTTTTCAGTGTTTATCGGGTCATTTGCCTTTTAACTGCATTGTGCTC
AGACGGCCTTGTCAAGGGTCATTTTTCTTACACACACAGCCTAGTTGAATTTTTCTCAATGTAA
CAGGAGGGTCAGGTGTTTGGTAAAGGGGTCTCTGAGGCTCCTCTCGACACCATTGTAAGATTCT
GTTATTTTCAGATTTGTGTATTTAATATATTAACTTAAGTGGATTAGGCCACTAAAACAAATTC
CCAGTCACTCCGACTCTGTGAGGTTACTTAAATAAATCCACAGTGGCCGGGCACCGTGGCTCAC
GCCTGTAATCCCAGCACTTTGGGAGGCCAAGGCGGGTGGATTACACGAGGTCAGGAGTTTGAGA
CCAGCCTGGCCAACATGGTGAAACCCTGTCTGTACTAAAAATGCAAAAATTATCCAGGCGTGGT
GGTGGGCGCCTGTAGTCCCAGCTACTCGGGAGGCTGAGGCAGGAGAATCGCTTGAACCCAGGAG
GGGGAGGTTGCAGTGAGCCAAGATTGCACCACTGCACTCTGGCCTGTGTGACAGAGTAAGACCT
GTCTCAAAATAATAATAATAATAATCCACAGTAAAATACAATCATTTCAGTCACTTTGACCATT
TTACGAGTCTGTTTTAAGGACTGACTGGTTGATTCCATGTTTGCTTCAAAATTGCCAGTTTAAT
CAGGGACTATTGGTAGGATTGCCCACAGACCACTTTTACAACAGGTAATTCAATCAGTTTAAAT
ATTGTATTTCCATTTTTTTTCCATCTGTTTTTGCAGCGCCAGCCAGCCTTTGACACCTTTGATG
GGTCCCTGTTTGCTGTTTTTCCTTCTCTAAATGAAGAGCAAACACTGCAAGAAGTGCCAACAGG
CTTGGATTCCATTTCTCATGGTAATTGGTTCCTCAGACTTGACAAATTGTGCATGATTTTCCTA
AGTAGTTCAGTTAATAAAGAGATGACAGTTCCTAAGTGAGAAATGTTGGTATCTGGAATCTTTG
ATATGAATAGCCAGAAGTTCTGGACCTGGGAGAAGGCAGTTTTTCATTTGTATCAACTTGGTGA
TTCTGATGTGGATTCTGATGTCTTGAGTGCCAGACGCCCCCCAGTGAGCCGCTGTTAGGAAACT
GCAGGGGAACCCCTGAGCTGCCCCTCCTGGATGTTTTCAACATTGGCAAGCAGGTCCTCATCTC
CCTGCTCTGCCCTGGAAAACAGACAGAAGAGTAGACATTAAAACACACCCAAACTTGTAATTTG
GGTTTGCATCATTAGTTAAATCATCTCCCCTGCCCTGAGTTTTGGGGCAACAAATTGTTCTTTG
TAATCCTTACCCATCGGTAGTTCTTTCTGTAGTTCTTCGTGTCATTGTTTATTCTTCTTAAAGG
AATAGCACGTCAGATAATGTTCATACAATTACTGTGACAGAGCTATTAGTTGGAAACATGCCAG
AGATGACTGTGTCATGTGAAGTAGGTGCTGAGAATACAGGCCAGAAAATTAATTAAAACGCATA
CACTGGCGTAAGCATAACCCTGCATGGCAGCCATTTATCATTCTTGAAATTGCTGAGGGTGTCT
GGAGAAGATTGTTTTTCAATAAAAATGGAAGGTGTGGTTCCATTACCGTGCTAAGTGCGGCACA
TTCATATCACACTCACTTCTCAGCTCTACGCCTAGTTTAATGGAAGTGACTTCCAAATGATACA
GTCCCTTCAGCCTTTTGACCAGAAACCATGCAACTGTTTTCATTGCCTACAATTTTAAAATAGA
CTTAGCAGTTTTCCCTACGCACAACTGCTTTGTGACGCTTTGCAAAATAGAATCAAATCTGAGT
TTTATTTTAGATTTTTTTAATAAGTATAGTTCTGAGATATATATTTTCTCAATTATAGTCAGAA
AAGTTTTTTGTTAATAGTTACACTGTTTTAAGGAATCATGCCAAGGTTTGAGATCAAAATTGTT
CTTTTCCAAAAACTAAGATGTCTCTCCTAAATCTCCACCTGATATCACCAACTTGAAGTCCTAA
TGTCCCCATGGGGGGTTTCCTTCCAGACTCCGCCAACTGTGAATTGCCTTTGTTAACCCCGTGC
AGCAAGGCTGTGATGAGTCAAGCCTTAAAAGCTACCTTCAGTGGCTTCAAAAAGGAACAGCGGC
GCCTGGGCATTCCAAAGAGTAAGTACTGCTTTCCTGAGCCTGCACTGGGTGAGAAGAACCAACT
TCAGTGCAGTTGTTTGATCTTGACTTGTTTATTAAGCTTTTGCTTGGGGTATTCTGCAAAGAGT
AGCATGGATGTCGTTAACCTGAGCCAGTTTCTGTTTCACCCCAATCAACCCAAGACTTGATCCA
GAACTCATTAAATATGTAGTAGAAGGACGCATTACTGGTGTCTTGAAATGTGCCTGGGTCGTGT
CAGAATGGTGACGTGTCATCATGGTATCTTGCTCATTCGTGGGTTCTGGTGTATGTCGGTACTT
GGTGCATAAATTAGGGATGACAGTGGTCTCACTACCTTTCCACTGTTTTTACCTCATGTGTTCC
ATTTTTTCTTCTCTCCTGGTAGACCCCTGGCTGTGGAGTGAGCAACAGGTATGCCAGTGGCTTC
TCTGGGCCACCAATGAGTTCAGTCTGGTGAACGTGAATCTGCAGAGGTTCGGCATGAATGGCCA
GATGCTGTGTAACCTTGGCAAGGAACGCTTTCTGGAGCTGGCACCTGACTTTGTGGGTGACATT
CTCTGGGAACATCTGGAGCAAATGATCAAAGGTACCAGCTGAACGTCTTACTTCTCCTTGTCCA
GGATGAGCTGTGGCCGGGAAGACTGATTGGGAAAGTCACGTGGGTGTTCTTCAACCTTAGGGTT
GCCACTTGAAATGACATAGAGTACCTTGCCTCAAAATGCCACTCAAGTGAGTCAGATATATGGC
AGTGATTATAGATTTTTATCCCACTTTATGTGAGTGTGTGTGTGTGTGTGTGTGTGTGTGTATA
GCATCAAGTATAGCCACAAGGTAGTAGCCTTAGTCACTAAATTGTTTGCTATTGCTGGCTGTAT
TCATCACGGGAGTCCATGTTGACTCAGATATGTAGGACAGCAAGAATTCCCACGTCTTCTGAGC
TCACCTTACAGAGCTAAGAGATATGGCAGCCTTAATGAAAGGGGGGAGCCTTTACTCACATAAA
CCCTGTGAATTCTACTGTAAATTTTCCTACATCCAAATGGAATGTATTTTGGATGTTGAGGATT
GTTGAGGGGGCGGGGTCTGTTTTTAAACAAGTGAGAAATTGATATATATTTACACGCTATCATA
TTGTACAGAAACTGGCCATTGATCAGTAGAAATCTCACCAAATACAGAGAAAGTCTTGATTCAA
AACTTAAAATAGCAGGGAGACCAATGTTTAAAACATGTTGAGTTAAAAAAGAAAAGAGGGCCAG
GTGCGGTGGCTCACACCTGTAATCCCAGCACTTTGGGAGGCGGAGGCGGGCAGATTACTTGAGG
TTAGGAGTTCAAGACCAGCCTAGCCAATATGGTGAAACCCTGTCTCTACTAAAAATACAAAAAA
TAAAAATATTAGCTGGGCATAGTGGCACATGCCTGTAATCCCAGATACTCGGAAGCCTGAGGCA
GGAGAATCCCTTGAGCCCGGGAGGCAGAGGTTGCAGTGAGTCGAGATCACACCACTGCACTCCA
GCCTGTGGGAGTGAGACTCTGTTGAAAGGAAGGAAGGAAGGAAGGAAGGAAGGAAGGAAGGAAG
GAAGGAAGGAAGGAAGGAAGGAAGGAAGGAAGGAAGGAGGGAGGGAGGGAAGGAAGGAAGGAAA
GAAAGAGGGAGGGAGGGAGGGGAGGGAGGGAGGGAGGGAGGGAGGGGAGGGAGGGAGGGAGGGA
GGGAGGGAAAGAAATAAAATACAGTAGGGATTATTTAGGTTAAAAAAATATAAGAAAATAAGCA
TGTTTATAGAATTCAGGGAACTAGTCCTCACCTGACTGATTTTAAACACACTGAATTTCAGCAT
CAACTGTAGACTCAGAATCTCGAAAACACCTAGGATTCAACAGCTAACTTTTTTTAGTTACGGC
GTCTGAGGGCCAGGGAAACTCATTTTTAAAGACCCTCATTTCTTCTGAACTTGGACAGGGCTTT
TCTACCAAGTTTCTGGTGATCTGTTTATTGGCAAATTGGGCCTGTTTTACCATTCCTCGGGCCT
GTGAGTTTGTGTGTATTTGGGTTTCCGGGTGGACGTGCACTTTGTAATATATTCGTAGTAAGTA
TGCATACACAGTGCACAAACCTTTTCCCTTTCAAAGTTCTTGGGCACATGTGTTCCCCATCCCA
GACTCCCACAGCCCTGGAGACTTCCCACACCCTGGGGAAGCGTGCAGGTTTGGAAGCAGTGGAA
CTGTAAGCTTCACGGGGGCAGACACCATCCATTTTATTAATTTCCTGTATGCAGCCTCTGGTAC
AGATGGCATTTCATAAATACTTAACTGTTTCTTGAATGCATGAATGAATTCAATACACCATCTG
GACCTTTTGCGATAGAAAATTTAGTGTTGCCAATGACATCATTCATTTTGGAAAATTTATTTTA
GTAATTGGAAAAATGGGTTGCTTTCTCTCGTTTTTTGAAAAAGACAGGCAGGGACGTGGAGCAC
CATGAGATTAAATGTGAAGCACTCCGTGCATCTCATCATTCTAAAATTATCATCTCACAGGAAT
TCAGAAAGTCAATTTGTTCTGTGTAGTTTTCACCTACATCTGCCATCTTACGTCTCCTGTGTCT
GCTTTTCAGAAAACCAAGAAAAGACAGAAGATCAATATGAAGAAAATTCACACCTCACCTCCGT
TCCTCATTGGATTAACAGCAATACATTAGGTCAGTCCGATTGATTCTGCCCTTAAGAACTTTGT
CTTCAGTCTTCCCAGTAGACTTGGAATCTCTCTACTGTAGGCTCCTAAGGGGCCACCTAGACCT
GTGTGTCTCTTATGGTGGCCACACGTGGGTGTTGAGCTATAAGCCATTGAAATGTGACTGGTCC
ACATCGAGATGTGTAGGAAGAGTATAGTCATGCACACCAGATTTGGAAGTCTTAGTATGAAACA
GAGAATATAAAATGTCTCCCTGATAATTTTTAATGTATTGATTACATATTGAAATGATAATTGC
TATATGAGGCCAAGTAAAATAAGAAAATTAACTTCACCTGTTTCTTTTTGCTTTTTTAATATGG
CCACTAGAACTTTCGCAGTTGCTTAAGTGGCTCACACGATCTTCCATAGGTCAGCACTGTTAGT
GCTAGTCCAGGCAATTCTTAGAGCAAGGAGCAGAACGAAGTGAGCTGATTACCGCAGTCACCAG
TAATGTTGGTTTAGTGTAGCAAACAAGAGGCGGCAGGCACAGAGTGCGTGAAAAGCAATTTGTA
AACTGGCTCTGAACTTGGGAGCTTAAAGAGCTGTCATGGAATCTGGGTCCTTTCTTCTGGTTGC
TGATACTGGGGAGGGCACAGGGTCTGGCCAGGCCAGCGAGGGAGGGGTCCTCGGAACAGCAGGC
TTGGGTGGTGTTGGTCCGGCCCAGTGTTTTCTCTCTGGCCCCAGCTCCACCATTCAGACCCGGT
GAAGCCCACCTCTCTGCCCCACTGGGGGCTTCCTGCTCAGGAGGTCTTTCTGAACATGACAGTG
GGACACCCTGTGCCTGCCTGAACCTGAATCAGAGTCAGAGAGCTTCCTTCCCTCTCCAGAAAAT
AAATTATTCTGCAATGAACAGAGACAGGAGCGGTGTGATGAAGGAGCAGAAAGCAGAGTGGGCT
GTGCCTCAGCACACAGGGTTCATGGCCTGGTTCTGCCCTTATGAATCACACAGTACATGACACG
TCACTTGGTCTGTCTGAGCCTTACTCTCCTCGGAAGCTAAAAGAGCCAGTGAGCCCTGTCTTCC
TGAAGGTCCCATGAAAGCACTAGGGTTGTAGAAATAGGAACATTGAGTCCTGCCTGCAGAGGCT
GCAGTCATTTTGGCAGAGGGCTGCTGGTTGAGAAGCTGAATGGTTTGAGAAGAAGGCCTGGTGA
CAGACCCCCACCAAAGCCCGGGTCTCCACTGAGGTTCTGCAGAGGGCTGGAGTGTGCGGAGTGC
TCACCTGTCATTTGCTCGTAGGGGTTAGTTACTGGGGTAACACTGACTTTAAGAGCTCTGCCGT
CCGATTGTTCTGTTCCAGGTTTTGGCACAGAGCAGGCGCCCTATGGAATGCAGACACAGAATTA
CCCCAAAGGCGGCCTCCTGGACAGCATGTGTCCGGCCTCCACACCCAGCGTACTCAGCTCTGAG
CAGGAGTTTCAGATGTTCCCCAAGTCTCGGCTCAGCTCCGTCAGCGTCACCTACTGCTCTGTCA
GTCAGGACTTCCCAGGCAGCAACTTGAATTTGCTCACCAACAATTCTGGTAAGATTGGAAGCAT
CTTTCAACAAGGCTGTTGCTTTGATTCTGAGAACCCCAGAGCCATAATGAACCTCTTAATAAAT
ACTTCCTGGATTCAGCCATTAGAGAAGGGGGTCAAAGCCCATGTTCTAAGTGGATTTCCAACAA
GCATACCCCTAATATGTTTCAGGCACTGGCCCAGGTACTGCAGGCATTGAGTAAACAAAACAGT
CCCTGCTCTTACAGCCAGATTTCTCCACACTTTGGGATATTTATGGAGGCGATGCAGGAATGTG
ACATTTCTTCTTATGGAGATGTAACATAAATACAGTGCACAAGTCTCAGGTCATGTGGCTGGTT
GGATTTGTGTATGTATGTATACCCATGTAATAACCATCCAGATGGAGATAGAGAAGTTTCCAGT
AGCCTGGAAGATTCCTTCGTACCCCTTCCCATCAATAATGTCCCCCCGACAGAAGGGAACGATT
CTGACTTTTATCATTACGGATTAGGTTTTGATTTTCTTGAACTTTACATAGATGGAATCATAAG
GCATGTTTTCTTTTGTGTTTGTCTTTTACTCATCATGTCTGGGATTCATGCATGTTGTTGGATG
TAGCAGCCATGTGTTCTTTTTCATCGCTGTATAGTATTCCATTATAAGAATTTTATTCATTCTA
CTGTTGATGGAAGATAGGATTTGTGTAGTATAGTTACCCTAAGGAATAAGCACCTGAGTAGAGA
GCATTTTGAGAAGTCTGAGTGGTTCTACACCAGCAGGTGCATGATTGCACTGGTAGTGGTTGGT
GATGCTATGGTCGTGCCCAAGACCAACTGTGTCCTGGAGGAATCAAAGTATGTGTTTGGTTGTC
TTTGCCAGGGACTCCCAAAGACCACGACTCCCCTGAGAACGGTGCGGACAGCTTCGAGAGCTCA
GACTCCCTCCTCCAGTCCTGGAACAGCCAGTCGTCCTTGCTGGATGTGCAACGGGTTCCTTCCT
TCGAGAGCTTCGAAGATGACTGCAGCCAGTCTCTCTGCCTCAATAAGCCAACCATGTCTTTCAA
GGATTACATCCAAGAGAGGAGTGACCCAGTGGAGCAAGGCAAACCAGTTATACCTGCAGCTGTG
CTGGCCGGCTTCACAGGTGTGTGTGGAACTCCGAGAGCCTGGCCGCCCAGTCTCCTGGGTCCTG
TCCCTTGCTTCCTTTCGAGCCACAGTACCACATTCACCGAGGGTGTTTCTAAGCTAGGTACACC
AGTGCTCTACACTCCATGTTTTATGCGTGGCTTGCTGTATCTCTGAATTCGACAAAAGCCACAC
TTGGAGGAATTTTCATGTACACAGAGCTCCATGGAATGAGTGAAAATTGACAGGCCACTCCCTC
CACCTGGGACGTTCTGAGGAATAGTTGGAGCAGGGGACATGGCCTGTGCAGTCTTCAGCCCTGA
CTCACCCACTCCAAGAATCTCAAAGAATGTAAACTGGCTTAGGGAGGGGCAGCAGAGCCAGTCT
CAAATAATATGGGCACTGTTTACCTTTTCTTGCTTCTAAAATCTATCTGCTCTTATTTTTGAGG
TTTGGGTGAGAGCTATTCTAGGCAGTGGACAAATATAAAGCTGTTTTTAGAAACAGGTATTTCC
AGTACTTTAGCGAAAATACTGGGGCTTTGTTTTCACTGCTTCGTGTATCCTTAAATGCGTTTTT
GTTTTTGTTTTTTTAACCATATGGAGCCACTGTGGATTGAAATTACCTAACTTAAAAAAATCTG
ATAAGTTTAAAATAAGGATACTGAGGCATTCATTCTTTTTCTGTCATCTAAGAACAATCAGCCA
CGTAAACGTATTAAAATACACGGAAAGAGTGTGTGAAAGCCATTATCGGTTTCATTATTGGGAG
TTTAGGGCCTGAGAAGAATAAACAGCCATAACGTTAGTGTGGTTCATTTTAATAGTAACATAAG
TTTCTGTTTATTGACTGCCAACTCTGTAGTTAAGTAGGCATACAGTCTGTAGGACAGCCTGGCA
TGGGGGTGGTATGAGGCCATTTTACAGATGGGGAAATTGGCTCACAGGCAGACAGCTATCGGTG
CTCACACAGCTCTGGTTCTAGAATGCCTGTCCCGTGCTTTACAGCAAAGCTTTATTGATAACGG
CCATTGCGTTTCTGAGGAAGAGCTATTTGGTAGAAAGAGGTTGGGCTTAGGGTCGGAAGATCTA
GGCTCTTCTCCTGTTTGCTGTGATGGGCGTGTGATCCAGGACAAGGCACCTGGCCTCTGAGCCG
CATCTTATCCCTCCTCTTTAGAACAGAACCACACTGACACCTGCTTTGCAAGGTGGTAGTTCTA
AATGTCAATCGAGGACAGGTGGACGAGAAGCTCTTGGCAAAGCATGCTTCACCAGACAGAGGCT
GTGAGGCAACGGGTGTGACCCCGCGTGGCTATTGAGTAAACCGGGCTCACATTAAGCAATTTAC
ACATGGTCATTTGGCAGGAAGGTGGCATGGGATCCCACGTATGACACGCCTCATTCTGTGATGA
AACACCCCCTCAGCCACCCCCATCTCTGCCCCACTGGGTGTTTCTAGAAAAAAACCTTGAAGGT
TTTTACATAAGGAACAATTAAACCAGATGGGGAATTTTAATGCCAAGAGTTGGTGGCCCTAAAC
TTTCTTCCAGAAAGAACAAACCTAGCAATCAAGGGGAAGAGTGTCTCGCCTAGTGACCTTATTT
TCTAGGAGTAGGCAGTGTGGAGCAGGAACTCACATTTGGTGCCCCGCCCCATCCCGTTAAAGCA
CTTAGTACTGTCACCAACACCTTGAGTGCCACCCTGAGTGTAACATCGGAACCCCATTCAGAGA
GTTGGGTCTGCATTCCTAAATCAGCATGTACAATTAGGATGGTTAAAGACTTGCTGTATTTTAC
ATCTGTGAAAGGGTATGATCCGTCTCCCTCCCTCTCCCCGCAGGAAGTGGACCTATTCAGCTGT
GGCAGTTTCTCCTGGAGCTGCTATCAGACAAATCCTGCCAGTCATTCATCAGCTGGACTGGAGA
CGGATGGGAGTTTAAGCTCGCCGACCCCGATGAGGTATGGCCAGAGCCCTGGGAAATCTCTGGG
CTTGAAAACCTGATTTCCTGCTTGCATTCAAAAACTCAGTTCTTTGGGCACAAAAAAGGGTTCA
CCAGTACTGCTGAGAATCTTTCCACGTGAGGCATCCTTGGCTGTTGGGAAAATGGAAGTGGAGT
CATTGCTTTGTTGATAAACGTGTACAGTGTTTTCTGGGTATGCTTCATACAAGGGCGTTGCACA
GATTTCACTGTCTTGATCAGTTGTCTGATCAAGAGGCCCAAGCTGCAAACTGAGGTTCTCTGCT
GACCATCTGAAAATGCCTTAGGAAGGCTGCCCTTGTCCATCGGGGGAACAAGCCTCATGTGGCC
CCGAGCCAGCCTTCTGTCCACTCCTTAGCTGAGCCCAGCAGTGTTGACCCCAGCCCTGCAGAGT
TAGAACCAGGAGCCCTGTCTAAAGGAGAAGGCCAAGGGCAGGTGGGATACCAACCCCGCCAGAA
AAGCGTTAGAGAGAAGCCCTTGGTCTCTGGCACTGGGGAGCGTAATCTGTCACCATTCCCACCC
CGCCTCCAGTTCCCTTGGGAGACAAGCATTGAGGAATGAGTATATCACACAGCTCAGAATCCCA
CTCGGCACCTGTTAACTTCAGTAGTTGGAATATCCAGCCTGGAGGGTGGGTGACACCACCTTTC
CCTGGTCTCAGGACCCTCCTGGCTCTGAACCCTTGGCTCCCAGGAGGTTTCACTGAGCTGGGGC
CAGGGAGCAGGGACCTCATTCCCCAGTGGTTCTGCCCCTTGGGGACACAGTGCCCCTACCATAG
GTACTCAAAGGTACTCAGAGGTACTCAAAAGGTCCTCCTGCGGACCTTGTGTAGGTGTCAAGTT
CTCTCTAGAGTGAACATGCCTCAGAATCATAATCAGGGAGGAATGTCATTCACTTTTTCTTCAT
TGACAAATTGAGTTTAACTCTTTTCCATCCATGTTCACCAAAGGTGGCCCGCCGGTGGGGAAAG
AGGAAAAATAAGCCCAAGATGAACTACGAGAAGCTGAGCCGGGGCTTACGCTACTATTACGACA
AGAACATCATCCACAAGACGTCGGGGAAGCGCTACGTGTACCGCTTCGTGTGCGACCTCCAGAA
CTTGCTGGGGTTCACGCCCGAGGAACTGCACGCCATCCTGGGCGTCCAGCCCGACACGGAGGAC
TGAGGTCGCCGGGACCACCCTGAGCCGGCCCCAGGCTCGTGGACTGAGTGGGAAGCCCATCCTG
ACCAGCTGCTCCGAGGACCCAGGAAAGGCAGGATTGAAAATGTCCAGGAAAGTGGCCAAGAAGC
AGTGGCCTTATTGCATCCCAAACCACGCCTCTTGACCAGGCTGCCTCCCTTGTGGCAGCAACGG
CACAGCTAATTCTACTCACAGTGCTTTTAAGTGAAAATGGTCGAGAAAGAGGCACCAGGAAGCC
GTCCTGGCGCCTGGCAGTCCGTGGGACGGGATGGTTCTGGCTGTTTGAGATTCTCAAAGGAGCG
AGCATGTCGTGGACACACACAGACTATTTTTAGATTTTCTTTTGCCTTTTGCAACCAGGAACAG
CAAATGCAAAAACTCTTTGAGAGGGTAGGAGGGTGGGAAGGAAACAACCATGTCATTTCAGAAG
TTAGTTTGTATATATTATTATAATCTTATAATTGTTCTCAGAATCCCTTAACAGTTGTATTTAA
CAGAAATTGTATATTGTAATTTAAAATAATTATATAACTGTATTTGAAATAAGAATTCAGACAT
CTGAGGTTTTATTTCATTTTTCAATAGCACATATGGAATTTTGCAAAGATTTAATCTGCCAAGG
GCCGACTAAGAGAAGTTGTAAAGTATGTATTATTTACATTTAATAGACTTACAGGGATAAGGCC
TGTGGGGGGTAATCCCTGCTTTTTGTGTTTTTTTGTTTGTTTGTTTGTTTGTTTTTGGGGGGTT
TTCTTGCCTTGGTTGTCTGGCAAGGACTTTGTACATTTGGGAGTTTTTATGAGAAACTTAAATG
TTATTATCTGGGCTTATATCTGGCCTCTGCTTTCTCCTTTAATTGTAAAGTAAAAGCTATAAAG
CAGTATTTTTCTTGACAAATGGCATATGTTTTCCACTTCTTTGCATGCGTTTAAGTCAGTTTAT
ACACAAAATGGATTTTATTTTTTAGTTTAACTGTGTTTCTCCGACAGCTCACCTCTCTCTGACC
ACCCAGCCATTTCCTTCCTGTGCTCCACGTTCTTCTGTGTGATTAAAATAAGAATATTATTTTT
GGAAATATGCAACTCCTTTTCAGAGATCAGGAGGGATTTATGTAGCAGCTATTTTTACTGCAAA
AGTAATTCACTGGAAAAAAAATGTAATTTGTAAGAAAGCTTTATTTTTATCTCAGCTCTATGTA
AAGTTAAAGTTACTGTACAGAGCTGAAGGACGGGGGGCGGTAGGGGTCTTGATGAAACCTCTTG
AACGAAGCACAGTTTGTCCCATCTTTGTTCACTCGTGTGTCTCAACCATCTTAATAGCATGCTG
CTCCTTTTTGCTCAGTGTCCACAGCAAGATGACGTGATTCTTATTTTCTTGGACACAGACTATT
CTGAGGCACAGAGCGGGGACTTAAGATGGGAAAGAGAAAGCATCGGAGCCATTCATTCGGAGAA
AACGTTTTGATCAAAATGGAGACTTTTGTAGTCGTTTCAAAAGAGCACCTGAGTCATGTGTATT
CCCGGCCTTTATAAATGACCCGGTCAAGTTGGTTTCAAAGTCCGACAGGCTTGTCTGTTTACTA
GCTGCGTGGCCTTGGACGGGTGGCTGACATCTGTAAAGAATCCTCCTGTGATGAAACTGAGGAA
TCGGGTGGCCGGGCAAGCTGGGAAGAGCAAAGCCAGAGCTGCGCTGCCTCAATACCCACAAAAG
ACCATTCCCAGTATACATAAGCACAGGATGTTTTTCTCAAGAGGGATGTATTTATCACTTGGAC
ATCTGTTTATAATATAAACAGACATGTGACTGGGAACATCTTGCTGCCAAAAGAATCCTAGGCA
GTGGCTCATTGTATGTGAGGTTGAACCACGTGAAATTGCCAATATTAGGCTGGCTTTTATCTAC
AAAGAAGGAGTTTCATGGGGTTCAGCCTAACAGTTATGGAAACTACAGTCCTTATAAACCATTG
GCATGGTAATAAACAGATCTTAAGTATAAAAATTTTGTAATTGGGCCTTTACTCTCTCAATAAT
AAAGTATTTTGTTTATATAAATTCTTTGTGATAGTCCTCGTTCTTCCTCTCCACACCCAGCATG
AAGGAGTTGGAGGAAGGATGTTAACCCCAGATCCATTCTCTACTCAAAACATTCCATCATCAAG
CGGCAAGTCTCTGTTTAACTGGTTTATACACAAGTCACTTAGAAACCACAACCCAAATTGGAAT
CAACTTTGAGCCCTTCCTAAAAGAATTCCCAAAAAGTGCTCTCTTTCAAAACAAAAAATTCTTT
TAAGAAAGTGTTATAATAGAAAGATTCAAATGTCTTTCTTTGCCAGAAGCTTGGCAGAGATAAC
AGAGGAGAGATTCTGGAATGTTTATATTAAGTACATTAGAATTTGAGGTTTAAGTACTTTTGGA
ACTGAGGCCACAACACTCTGTCCCCTCAGTGGAGTCTGACATTGGTAAGGTGATGGTGGTCTTA
GGACACCTGTTTTCAAATTTGTTCTCTTTACATGCTCCAAAATTAGTGAGTATGCCAAAAAGCT
TATATTCATTGGTGCGCTAGAGCCAGCTCACTGTGTTCTCCCGAGAGCTACATGTCCCTTCCCA
TCATCGCATCAGTAATGGTAGCTTGAGATGGGGCGTGATGAGAGT

In some embodiments, the ETS2 inhibitor comprises a small interfering RNA (siRNA) molecule comprising a sense strand. In some embodiments, the sense strand consists of 15 to 30 linked nucleosides. In some embodiments, the sense strand comprises a sequence having at least 95% identity to an equal length portion of a pregenomic RNA and/or an mRNA encoding Erythroblast Transformation Specific proto-oncogene 2 (ETS2). In some embodiments, the sense strand comprises a sequence having 100% identity to an equal length portion of a pregenomic RNA and/or an mRNA encoding Erythroblast Transformation Specific proto-oncogene 2 (ETS2). In some embodiments, the small interfering RNA (siRNA) molecule comprises an antisense strand. In some embodiments, the antisense strand is at least 80%, at least 90%, at least 95% or at least 99% complementary to the sense strand. In some embodiments, the antisense strand is fully complementary to the sense strand.

In some embodiments, the ETS2 inhibitor is capable of inhibiting the expression of ETS2 in vitro by at least 50%, at least 60%, at least 70%, at least 80%, at least 90%, at least 95% or at least 99%. In some embodiments, the chr21q22 enhancer inhibitor is capable of inhibiting the expression of ETS2 in vitro by at least 50%, at least 60%, at least 70%, at least 80%, at least 90%, at least 95% or at least 99%.

In some embodiments, the ETS2 inhibitor comprises an antisense nucleic acid molecule. In some embodiments, the antisense compound is an antisense oligonucleotide. In some embodiments, the antisense compound specifically binds to an ETS2 mRNA sequence comprising SEQ ID NO:2 or SEQ ID NO:3, or an ETS2 genomic DNA sequence comprising SEQ ID NO:5. In some embodiments, the antisense compound specifically binds to an ETS2 mRNA sequence comprising SEQ ID NO:2.

In some embodiments, the antisense oligonucleotide comprises at least one modified internucleoside linkage. In some embodiments, the modified internucleoside linkage is a phosphorothioate linkage. In some embodiments, the antisense oligonucleotide comprises at least one modified sugar moiety. In some embodiments, the modified sugar moiety is a 2′-O-methoxyethyl sugar moiety. In some embodiments, the antisense oligonucleotide comprises at least one modified nucleobase. In some embodiments, the modified nucleobase is a 5-methylcytosine. In some embodiments, the antisense oligonucleotide is a chimeric oligonucleotide.

In some embodiments, the ETS2 inhibitor comprises a compound comprising an ETS2 inhibitor and a conjugate group. In some embodiments, the chr21q22 enhancer inhibitor comprises a compound comprising a chr21q22 enhancer inhibitor and a conjugate group. In some embodiments, the conjugate group comprises one or more antibodies or antigen-binding portions thereof, for example a Fab fragment. In some embodiments, the conjugate group comprises one or more carbohydrates. In some embodiments, the conjugate group comprises one or more galactose moieties, one or more lactose moieties, one or more N-Acetyl-Galactosamine moieties, and/or one or more mannose moieties. In some embodiments, the conjugate group comprises one or more N-Acetyl-Galactosamine moieties.

In some embodiments, the conjugate group comprises a nanoparticle for delivery of the ETS2 or chr21q22 enhancer inhibitor to a macrophage. In some embodiments, the conjugate group comprises a lipid carrier. In some embodiments, the lipid carrier comprises one or more components selected from the list consisting of: poly(lactic acid) (PLA) microspheres, poly(D,L-lactic-coglycolic-acid) (PLGA) microspheres, liposomes, lipid nanoparticles, micelles, inverse micelles, lipid cochleates, and lipid microtubules.

The present invention provides a method of providing a diagnosis or prognosis of an inflammatory or autoimmune disease in a subject based on the expression status of Erythroblast Transformation Specific proto-oncogene 2 (ETS2). The present invention provides a method of providing a diagnosis or prognosis of an inflammatory or autoimmune disease in a subject based on the expression status of the chr21q22 enhancer (SEQ ID NO:1).

In some embodiments, determining the expression status of ETS2 or the chr21q22 enhancer comprises determination of one or more statuses in a biological sample obtained from the subject selected from the list consisting of: ETS2 mRNA levels, ETS2 protein levels, ETS2 DNA methylation status, ETS2 epigenetic status (such as histone marking, RNA changes or conformation changes), chr21q22 enhancer DNA methylation status and chr21q22 enhancer epigenetic status (such as histone marking, RNA changes or conformation changes). In some embodiments, determining the expression status of ETS2 comprises determination of ETS2 mRNA levels or ETS2 protein levels. In some embodiments, determining the expression status of ETS2 comprises the step of quantifying the expression status of an RNA transcript or cDNA molecule and wherein the expression status of the RNA or cDNA is quantified using any one or more of the following techniques: microarray analysis, real-time quantitative PCR, DNA sequencing, RNA sequencing, Northern blot analysis, in situ hybridisation and/or detection and quantification of a specific binding molecule (e.g. an antibody). In some embodiments, diagnostic methods of the invention further comprise a step of comparing or normalising the expression status of ETS2 with the expression status of a reference gene.

The present invention provides a method of treating or preventing a disease in a subject, the method comprising administering an inhibitor of a protein that activates Erythroblast Transformation Specific proto-oncogene 2 (ETS2) to the subject. In some embodiments, the protein that activates Erythroblast Transformation Specific proto-oncogene 2 (ETS2) is selected from the list consisting of: MEK, HSP90, RAF, SRC and ERK. In some embodiments, the MEK inhibitor is selected from the list consisting of Selumetinib, Trametinib and Cobimetinib, preferably wherein the MEK inhibitor is Selumetinib. In some embodiments, the inhibitor of a protein that activates Erythroblast Transformation Specific proto-oncogene 2 (ETS2) is an antibody-drug conjugate, an antibody-RNA conjugate, an esterase-sensitive motif-conjugated drug or a nanobody-drug conjugate. In some embodiments, the antibody-drug conjugate comprises an anti-CD163 antigen binding molecule, optionally wherein the antigen binding molecule is an antibody. In some embodiments, the antibody-drug conjugate comprises a cleavable linker, optionally wherein the cleavable linker is glucuronide-based (e.g. GlyPro). In some embodiments, the antibody-drug conjugate comprises a non-cleavable linker, optionally wherein the non-cleavable linker is maleimide-PEG3-based. In some embodiments, the esterase-sensitive motif-conjugated drug comprises cyclopentyl L-leucinate or cyclopentyl(S)-2-amino-2-cyclohexylacetate. In some embodiments, the esterase-sensitive motif-conjugated drug comprises a linker. In some embodiments, the linker is one or two carbons in length. In some embodiments, the subject has an inflammatory disease and/or autoimmune disease. In some embodiments, the inflammatory disease is selected from the list consisting of: Crohn's disease, ulcerative colitis, inflammatory bowel disease (Crohn's disease and/or ulcerative colitis), ankylosing spondylitis, primary sclerosing cholangitis, Takayasu arteritis and rheumatoid arthritis.

The present invention provides an inhibitor of a protein that activates Erythroblast Transformation Specific proto-oncogene 2 (ETS2) for use in a method of treating or preventing a disease in a subject, the method comprising administering an inhibitor of a protein that activates Erythroblast Transformation Specific proto-oncogene 2 (ETS2) to the subject. In some embodiments, the protein that activates Erythroblast Transformation Specific proto-oncogene 2 (ETS2) is selected from the list consisting of: MEK, HSP90, RAF, SRC and ERK. In some embodiments, the MEK inhibitor is selected from the list consisting of Selumetinib, Trametinib and Cobimetinib, preferably wherein the MEK inhibitor is Selumetinib. In some embodiments, the inhibitor of a protein that activates Erythroblast Transformation Specific proto-oncogene 2 (ETS2) is an antibody-drug conjugate, an antibody-RNA conjugate, an esterase-sensitive motif-conjugated drug or a nanobody-drug conjugate. In some embodiments, the antibody-drug conjugate comprises an anti-CD163 antigen binding molecule, optionally wherein the antigen binding molecule is an antibody. In some embodiments, the antibody-drug conjugate comprises a cleavable linker, optionally wherein the cleavable linker is glucuronide-based (e.g. GlyPro). In some embodiments, the antibody-drug conjugate comprises a non-cleavable linker, optionally wherein the non-cleavable linker is maleimide-PEG3-based. In some embodiments, the esterase-sensitive motif-conjugated drug comprises cyclopentyl L-leucinate or cyclopentyl(S)-2-amino-2-cyclohexylacetate. In some embodiments, the esterase-sensitive motif-conjugated drug comprises a linker. In some embodiments, the linker is one or two carbons in length. In some embodiments, the subject has an inflammatory disease and/or autoimmune disease. In some embodiments, the inflammatory disease is selected from the list consisting of: Crohn's disease, ulcerative colitis, inflammatory bowel disease (Crohn's disease and/or ulcerative colitis), ankylosing spondylitis, primary sclerosing cholangitis, Takayasu arteritis and rheumatoid arthritis.

The present invention provides a method of reducing macrophage activation by contacting said macrophage with an inhibitor of a protein that activates Erythroblast Transformation Specific proto-oncogene 2 (ETS2). The present invention provides a method of reducing proinflammatory cytokine production by contacting a macrophage with an inhibitor of a protein that activates Erythroblast Transformation Specific proto-oncogene 2 (ETS2). The present invention provides a method of reducing reactive oxygen species (ROS) production by contacting a macrophage with an inhibitor of a protein that activates Erythroblast Transformation Specific proto-oncogene 2 (ETS2). The present invention provides a method of reducing macrophage phagocytosis by contacting said macrophage with an inhibitor of a protein that activates Erythroblast Transformation Specific proto-oncogene 2 (ETS2). The present invention provides a method of reducing macrophage migration by contacting said macrophage with an inhibitor of a protein that activates Erythroblast Transformation Specific proto-oncogene 2 (ETS2). The present invention provides an inhibitor of a protein that activates Erythroblast Transformation Specific proto-oncogene 2 (ETS2) for use in a method of reducing macrophage activation by contacting said macrophage with an inhibitor of a protein that activates Erythroblast Transformation Specific proto-oncogene 2 (ETS2). The present invention provides an inhibitor of a protein that activates Erythroblast Transformation Specific proto-oncogene 2 (ETS2) for use in a method of reducing proinflammatory cytokine production by contacting a macrophage with an inhibitor of a protein that activates Erythroblast Transformation Specific proto-oncogene 2 (ETS2). The present invention provides an inhibitor of a protein that activates Erythroblast Transformation Specific proto-oncogene 2 (ETS2) for use in a method of reducing reactive oxygen species (ROS) production by contacting a macrophage with an inhibitor of a protein that activates Erythroblast Transformation Specific proto-oncogene 2 (ETS2). The present invention provides an inhibitor of a protein that activates Erythroblast Transformation Specific proto-oncogene 2 (ETS2) for use in a method of reducing macrophage phagocytosis by contacting said macrophage with an inhibitor of a protein that activates Erythroblast Transformation Specific proto-oncogene 2 (ETS2). The present invention provides an inhibitor of a protein that activates Erythroblast Transformation Specific proto-oncogene 2 (ETS2) for use in a method of reducing macrophage migration by contacting said macrophage with an inhibitor of a protein that activates Erythroblast Transformation Specific proto-oncogene 2 (ETS2).

In some embodiments, the protein that activates Erythroblast Transformation Specific proto-oncogene 2 (ETS2) is selected from the list consisting of: MEK, HSP90, RAF, SRC and ERK. In some embodiments, the MEK inhibitor is selected from the list consisting of Selumetinib, Trametinib and Cobimetinib, preferably wherein the MEK inhibitor is Selumetinib.

The present invention provides a method of screening for agents that reduce macrophage activation, the method comprising:
(a) contacting a macrophage with a candidate agent; and
(b) determining the expression status of ETS2;
wherein agents that reduce expression of ETS2 are identified as agents that reduce macrophage activation.

In some embodiments, determining the expression status of ETS2 comprises determination of ETS2 mRNA levels or ETS2 protein levels.

The present invention provides a method of screening for candidate genes involved in macrophage activation, the method comprising:
(a) introducing to a macrophage a CRISPR nuclease system comprising a CRISPR-associated protein (Cas protein) and a guide RNA (gRNA) that specifically binds to an ETS2 genomic nucleic acid sequence;
(b) measuring the expression status of one or more candidate genes; and
(c) identifying genes as being associated with macrophage activation by comparing the expression status to a reference expression status of the same genes from a control cell without the CRISPR nuclease system, wherein genes that have increased or decreased expression relative to the reference expression status are associated with macrophage activation.

In some embodiments, the ETS2 genomic nucleic acid sequence comprises the sequence of SEQ ID NO:5.

The present invention provides a method of screening for candidate genes involved in macrophage activation, the method comprising:
(a) introducing to a macrophage a CRISPR nuclease system comprising a CRISPR-associated protein (Cas protein) and a guide RNA (gRNA) that specifically binds to a chr21q22 enhancer nucleic acid sequence;
(b) measuring the expression status of one or more candidate genes; and
(c) identifying genes as being associated with macrophage activation by comparing the expression status to a reference expression status of the same genes from a control cell without the CRISPR nuclease system, wherein genes that have increased or decreased expression relative to the reference expression status are associated with macrophage activation.

In some embodiments, the chr21q22 enhancer nucleic acid sequence comprises the sequence of SEQ ID NO: 1.

The present invention provides a method of screening for candidate genes involved in macrophage activation, the method comprising:
(a) introducing to a macrophage a CRISPR nuclease system comprising a CRISPR-associated protein (Cas protein) and a guide RNA (gRNA) that specifically binds to the genomic nucleic acid sequence a candidate gene;
(b) measuring the expression status of ETS2; and
(c) identifying genes as being associated with macrophage activation by comparing the expression status of ETS2 to a reference expression status of ETS2 from a control cell without the CRISPR nuclease system, wherein candidate genes that modulate ETS2 expression status are associated with macrophage activation.

In some embodiments, the expression status of the one or more candidate genes is quantified using any one or more of the following techniques: microarray analysis, real-time quantitative PCR, DNA sequencing, RNA sequencing, Northern blot analysis, in situ hybridisation and/or detection and quantification of a specific binding molecule (e.g. an antibody).

In some embodiments, the antibody-drug conjugate comprises an antigen binding molecule that specifically binds a macrophage marker, optionally wherein the antigen binding molecule is an antibody. In some embodiments, the antibody-drug conjugate comprises an anti-CD209 (DC-SIGN) or an anti-CD206 (MRC1) antigen binding molecule, optionally wherein the antigen binding molecule is an antibody. In some embodiments, the antibody-drug conjugate has an antibody: drug ratio of at least 1:2, at least 1:4, at least 1:6, at least 1:8 or at least 1:10. In some embodiments, the ETS2 inhibitor or chr21q22 enhancer inhibitor comprises an agent that disrupts the function of co-transcriptional activators (e.g. BRD4 or CDKs). In some embodiments, the ETS2 inhibitor or chr21q22 enhancer inhibitor comprises an agent that targets chromatin regulators (e.g. readers, writers and erasers of chromatin modifications). In some embodiments, the ETS2 inhibitor or chr21q22 enhancer inhibitor comprises an agent that targets non-coding RNA at the ETS2 locus or at the chr21q22 enhancer locus. In some embodiments, the chr21q22 enhancer inhibitor comprises a genetic editing technique selected from the list consisting of: gene/base/PRIME editing strategies (for example CRISPR-based genome-targeting tools), gene therapies (for example Adeno-Associated Viruses (AAV), retro- or lenti-viral vectors) and RNA therapies (including, but not limited to, antisense oligonucleotides).

### BRIEF DESCRIPTION OF THE DRAWINGS

FIG. 1-Resolving molecular mechanisms at chr21q22. A. Annotation of the disease-associated chr21q22 locus, depicting the IBD genetic association, physical interactions of the disease-associated haplotype in macrophages (promoter-capture Hi-C data), and H3K27ac ChIP-seq data from various immune cell-types. B. Schematic of experiment for determining the function of the chr21q22 locus in monocyte-derived macrophages polarised under chronic inflammatory (“TPP”) conditions. C. Histograms depicting the expression of ETS2, BRWD1, and PSMG1 during inflammatory macrophage polarisation, as measured using PrimeFlow RNA assays to directly quantify RNA by flow cytometry. Data are representative of one of four donors. D. Relative ETS2, BRWD1, and PSMG1 expression in chr21q22-edited inflammatory macrophages (relative to non-targeting control cells; NTC). Plot shows log 2 fold-change in mean fluorescence intensity (n=4, data represent mean=/-SEM, two-way ANOVA). E. SuSiE fine-mapping posterior probabilities of IBD-associated SNPs at the 21q22 locus (99% credible set). F. MPRA at the chr21q22 locus depicting oligonucleotide coverage (top), enhancer activity in inflammatory macrophages (analysed using a sliding window analysis of tiling oligos; middle), and expression-modulating effects of candidate SNPs within the identified enhancer site (bottom) (n=8). Shaded region in enhancer activity plot depicts region of significant enhancer activity. G. PU.1 ChIP-seq peaks at the chr21q22 locus in macrophages. H. BaaIChIP analysis of allele-specific PU. 1 binding at rs2836882 in two heterozygous macrophage datasets (data represent 95% posterior distribution of allelic binding ratio) I. H3K27ac ChIP-seq data from major (top) or minor (bottom) allele homozygotes at the chr21q22 locus. Data are representative of two of four donors.

FIG. 2—ETS2 is necessary for macrophage inflammatory responses. A. Schematic of experiment for CRISPR-editing primary monocytes and differentiating monocyte-derived macrophages under chronic inflammatory (“TPP”) conditions. B. Macrophage cytokine secretion following ETS2 disruption. Heatmap shows log 2 fold-change of cytokine concentrations in the supernatants of ETS2-edited macrophages relative to unedited macrophages transfected with a non-targeting control gRNA (NTC). n=8, Wilcoxon matched-pairs test, two-tailed. C. Histogram depicting phagocytosis of fluorescently-labelled zymosan particles by ETS2-edited and unedited macrophages (left). Data representative of one of seven donors. Phagocytosis index in ETS2-edited and unedited macrophages (calculated as product of proportion and mean fluorescence intensity of phagocytosing cells; right). Plot shows log 2 fold-change in phagocytosis index for ETS2-edited relative to unedited macrophages (Wilcoxon signed-rank test, two-tailed; data represent mean±/−SEM). D. Production of ROS by ETS2-edited and unedited inflammatory macrophages (measured in relative light units; left). Data representative of one of six donors. Western blot for gp91phox, p22phox, and EROS expression in ETS2-edited and unedited inflammatory macrophages (right). Data representative of one of three donors. E. Volcano plot of differentially-expressed genes in ETS2-edited versus unedited (NTC) inflammatory macrophages (limma with voom transformation, n=8). F. Gene set enrichment analysis (fGSEA) of differentially-expressed genes between ETS2-edited and unedited inflammatory macrophages. Results of selected Gene Ontology Biological Pathways shown. Dot size represents P-value and colour denotes normalised enrichment score (NES). G. Enrichment of differentially-expressed genes following deletion of the disease-associated chr21q22 locus (upregulated genes, top; downregulated genes, bottom) in ETS2-edited versus unedited macrophages. * P<0.05, ** P<0.01.

FIG. 3—ETS2 orchestrates macrophage inflammatory responses. A. Schematic of ETS2 overexpression experiment. Resting (M0) human macrophages were transfected with pre-defined amounts of in vitro transcribed ETS2 mRNA or control mRNA (ETS2 reverse complement), activated with low dose LPS (1 ng/ml), and harvested. B. ETS2 mRNA levels in macrophages transfected with ETS2 or control mRNA or untransfected (from separate experiment). C. Cytokine secretion following ETS2 overexpression. Plot shows log 2 fold-change of cytokine concentrations in macrophage supernatants (ETS2 relative to control) following transfection with 500 ng mRNA. D. Gene set enrichment analysis (fGSEA) of differentially-expressed genes between ETS2-overexpressing and control macrophages. Results shown for the same Gene Ontology Biological Pathways that were negatively enriched following ETS2 editing. Dot size represents P-value and colour denotes normalised enrichment score (NES), and border colour indicates mRNA dose. E. Enrichment of a disease-associated inflammatory macrophage gene set, derived from single cell RNA-seq of Crohn's disease intestinal biopsies, in ETS2-overexpressing macrophages (relative to control; top). Heatmap of leading-edge genes showing log 2 fold-change of gene expression in ETS2-overexpressing macrophages relative to control (500 ng mRNA; bottom). F. SNPsea analysis of enrichment of 241 IBD-associated loci within ETS2-regulated genes (red) and pathways previously linked to IBD pathogenesis (black). Significantly enriched pathways (Bonferroni-corrected permutation P<0.05) indicated by §. * P<0.05, ** P<0.01.

FIG. 4—ETS2 directs macrophage responses via transcriptional and metabolic effects. A. Genes co-expressed with ETS2 in 64 monocyte-derived macrophage datasets. Dotted line equivalent to FDR P<0.05. B. Effect of ETS2 disruption on glucose metabolism. Colour denotes median log 2 fold-change in label incorporation from 13C-glucose in ETS2-edited cells relative to unedited cells. Bold black border denotes P<0.05 (Wilcoxon matched-pairs, two-tailed, n=6). C. Gene set enrichment analysis (fGSEA) of differentially-expressed genes between ETS2-edited and unedited TPP macrophages, treated with either roxadustat or vehicle. Results for selected Gene Ontology Biological Pathways shown. D. Enrichment heatmap of ETS2 CUT&RUN peaks (IDR cut-off 0.01, n=2) in accessible chromatin from TPP macrophages (4-kb regions centred on ATAC-seq peaks). E. Features of ETS2 binding sites (based on gene coordinates and H3K27ac ChIP-seq in TPP macrophages). F. Enrichment of an ETS2 binding motif in ETS2 CUT&RUN peaks (hypergeometric P-value). G. ETS2 binding, chromatin accessibility (ATAC-seq), and enhancer activity (H3K27ac) at selected loci. H. UpSet plot of intersections between ETS2 gene lists, including genes with ETS2 peaks in their core promoters or cis-regulatory elements and significantly up- (Up) or down-regulated (Dn) genes following ETS2 editing (KO) or overexpression (OE). Vertical bars denote shared genes between lists, indicated by connected dots in lower panel. Horizontal bars denote proportion of gene list within intersections. I. ETS2 binding, chromatin accessibility (ATAC-seq), and enhancer activity (H3K27ac) at the disease-associated chr21q22 locus.

FIG. 5—ETS2-driven inflammation is evident in disease and can be therapeutically targeted. A. Enrichment of chr21q22-regulated genes within IBD intestinal macrophages (top), ankylosing spondylitis synovium (middle), and primary sclerosing cholangitis liver (bottom). All relative to unaffected control tissue from the same datasets. Gene set comprised significantly downregulated genes following chr21q22 deletion. B. Candidate drug classes (from NIH LINCS database) that phenocopy the transcriptional consequences of ETS2 disruption. C. Volcano plot of fGSEA results for NIH LINCS drug signatures (FDR P estimated using adaptive multi-level split Monte-Carlo scheme; NES, normalised enrichment score). Significantly enriched MEK inhibitor genesets coloured by molecule. D. Schematic of the known upstream signalling pathway that regulates ETS-family transcription factors. E. Schematic of experiment for in vitro MEK inhibitor (PD-0325901) testing in inflammatory macrophages (TPP). F. Heatmap showing relative expression (log 2 fold-change) of chr21q22-regulated genes in inflammatory macrophages following MEK inhibitor treatment (compared to vehicle control, n=3). G. Gene set enrichment analysis (fGSEA) of differentially-expressed genes between MEK inhibitor-treated and control inflammatory macrophages. Results shown for the same Gene Ontology Biological Pathways that were negatively enriched following ETS2 editing. Dot size represents P-value and colour denotes NES. H. Cytokine secretion from IBD mucosal biopsies cultured with vehicle control, PD-0325901, or infliximab I. Estimation plot of GSVA enrichment scores for chr21q22-downregulated genes in IBD intestinal biopsies following MEK inhibition (MEKi). Error bars indicate 95% CI. J. GSVA enrichment scores of a biopsy-derived molecular inflammation score (bMIS). Data in h and i represent mean±/−SEM. Wilcoxon matched-pairs test, two-tailed, n=10 (h), n=9 (j). * P<0.05, ** P<0.01, *** P<0.001, **** P<0.0001.

FIG. 6—Disease-associated variation at the chr21q22 locus is correlated with levels of ETS2 expression in monocytes. A. The risk haplotype on chr21q22 is an eQTL for ETS2 expression in resting and activated monocytes (data from Fairfax et al, 2014). B. Manhattan plots depicting the associations at chr21q22 with IBD (upper panel), eQTL in resting monocytes (middle panel) and eQTL in LPS-stimulated monocytes (lower panel). C. The IBD chr21q22 association colocalises with an eQTL signal in resting but not activated monocytes. PP3, probability that GWAS and eQTL signals are independent. PP4, probability that a shared variant is responsible for both associations.

FIG. 7—CRISPR/Cas9 editing of the chr21q22 locus and ETS2 in monocytes. A. Cas9 gRNAs were designed to flank the enhancer region within the chr21q22 locus at the indicated sites. Enhancer activity shown from H3K27ac ChIP-sequencing. B. Representative bioanalyzer trace of PCR amplicon from the target region in macrophages following transfection with equimolar mix of Cas9 RNPs containing either 5′ or 3′ gRNAs. Method for calculating editing efficiency shown with example calculation. C. Editing efficiency at the chr21q22 locus. Mean rate of deletion: 42.4%. D. Location and sequence of gRNAs used to edit ETS2. E. Editing efficiency at the ETS2 locus. Mean indel rates: gRNA1, 89.7%; gRNA2, 78.6%. F. Viability of macrophages following nucleofection with Cas9 RNPs and differentiation. Mean viabilities: NTC, 97.9%; gRNA1: 98.3%; gRNA2, 98.6%. G. Expression of myeloid lineage markers following ETS2 editing and TPP differentiation. Data represent mean±/−SEM.

FIG. 8—TPP macrophages recapitulate the transcriptional signature of monocytes and macrophages in IBD.

FIG. 9—MPRA in primary human macrophages. A. Schematic of MPRA. A library of oligonucleotides (each containing a genomic sequence and unique barcode) is cloned into a pGL4.10M cloning vector, and then a promoter and reporter gene are inserted using directional cloning. The resulting plasmid is transfected into TPP macrophages and RNA is extracted after 24 hours. After high-throughput sequencing, mRNA barcode counts are normalised to their corresponding counts in the input DNA library to assess expression-modulating activity. B. Identification of suitable promoters for MPRA in TPP macrophages. TPP macrophages were transfected with a reporter vector, with GFP expression under the control of different promoters. GFP expression was quantified by flow cytometry 24 hours later. C. Adapted MPRA vector for use in primary human macrophages, containing RSV promoter. D. Principal component analysis of element counts (sum of barcodes tagging same genomic sequence) in mRNA from TPP macrophages from 8 donors (red) and DNA vector (black). E. Heat map showing pairwise correlation of expression-modulating activity of all constructs between donors.

FIG. 10—Functional consequences of allelic variation at rs2836882. A. Schematic of PU.1 ChIP-genotyping assay to assess allele-specific PU.1 binding at rs2836882. B. Schematic representation of lines of best fit for various ratios of risk and non-risk containing DNA sequences. C. Standard curve generated using different allelic ratios of 200-nt DNA geneblocks centred on either the major (risk) or minor (non-risk) allele. D. Allele-specific PU.1 binding at rs2836882 in TPP macrophages demonstrating increased PU.1 binding in the presence of the risk allele (one-sample t-test, two-tailed). Data represent 95% confidence interval. E. Rank Ordering of Super-Enhancers (ROSE) analysis of H3K27ac ChIP-sequencing data from TPP macrophages from major-(left) and minor-(right) allele homozygotes demonstrating that the disease-associated chr21q22 locus meets the criteria for super-enhancer activity in major (risk) allele homozygotes, but not in minor allele homozygotes.

FIG. 11—Deletion of the chr21q22 disease-associated enhancer phenocopies ETS2 disruption. A. Cytokine secretion from TPP macrophages following editing at the chr21q22 locus. Heatmap shows log 2 fold-change of cytokine concentrations in the supernatants of chr21q22-edited cells relative to non-targeting control (NTC) cells (n=7, Wilcoxon matched-pairs signed rank test, one-tailed). B. Extracellular ROS production by NTC, chr21q22-edited, and ETS2 g1-edited TPP macrophages-quantified by a chemiluminescence assay. Points represent log 2 fold-change of area under curve (AUC) for edited cells versus NTC (Wilcoxon signed-rank test). C. Representative histogram demonstrating phagocytosis of fluorescently-labelled zymosan particles by NTC and chr21q22-edited TPP macrophages. D. Phagocytosis index for NTC and chr21q22-edited TPP macrophages. Phagocytosis index is calculated as proportion of positive cells multiplied by mean fluorescence intensity of positive cells (488 nm channel). Plot shows log 2 fold-change in chr21q22-edited cells versus NTC cells (Wilcoxon signed-rank test). Data represent mean±/−SEM. * p<0.05.

FIG. 12—Optimisation of mRNA-based overexpression in primary human macrophages. Primary human macrophages (M0) were transfected with different amounts of GFP mRNA containing modified nucleotides using Lipofectamine MessengerMAX. GFP expression was quantified by flow cytometry 18 hours after transfection.

FIG. 13—Metabolic effects of ETS2 disruption. A and B. Changes in total metabolite abundance (A) and percentage of label incorporation from 13C-glucose (B) following ETS2 editing in TPP macrophages. Colour shows log 2 fold-change in abundance or incorporation in ETS2-g1-edited macrophages relative to macrophages transfected with non-targeting control (NTC) RNPs. Bold black border denotes significant change (p<0.05, Wilcoxon matched-pairs signed rank test, two-tailed). C. Heatmap summarising metabolic changes following ETS2 disruption. Colour shows log 2 fold-change in metabolite abundance or label incorporation from ¹³C-glucose in ETS2 g1-edited relative to NTC cells (Wilcoxon matched-pairs signed rank test, two-tailed). * p<0.05.

FIG. 14—Functional studies in roxadustat-treated macrophages. A. Phagocytosis index in ETS2-edited or non-targeting control (NTC) TPP macrophages treated with roxadustat or vehicle. Phagocytosis index is calculated as proportion of positive cells multiplied by mean fluorescence intensity of positive cells (488 nm channel). Plot shows log 2 fold-change in edited versus NTC cells. B. Extracellular ROS production by ETS2-edited or NTC TPP macrophages treated with roxadustat or vehicle—quantified by a chemiluminescence assay. Points represent log 2 fold-change of area under curve (AUC) for edited versus NTC cells. Data represent mean±/−SEM.

FIG. 15—Exemplary ETS2 nucleic acid and amino acid sequences. SEQ ID NO:1-Exemplary chr21q22 enhancer sequence, hg19: chr21: 40466236-40466677; SEQ ID NO:2-Exemplary ETS2 mRNA sequence (NM005239.6) and SEQ ID NO:4-Exemplary ETS2 amino acid sequence (Uniprot: P15036).

FIG. 16—The transcriptional signature of ETS2 is detectable in affected tissues from chr21q22-linked diseases. A. Gene set enrichment analysis (fGSEA) of ETS2-regulated genes within ankylosing spondylitis synovium (compared to control synovium). B. fGSEA of ETS2-regulated genes within primary sclerosing cholangitis liver biopsies (compared to control liver biopsies). C. fGSEA of ETS2-regulated genes within intestinal macrophages isolated from patients with active inflammatory bowel disease (compared to healthy control intestinal macrophages). ETS2-regulated genes in A, B, and C were defined as those significantly downregulated following ETS2 editing with gRNA1-containing RNPs (upper panel) or gRNA2-containing RNPs (lower panel).

FIG. 17—Effect of MEK1/2 inhibition on ETS2-regulated genes. A, B and C. Gene set enrichment analysis (fGSEA) of gene sets upregulated (upper panel) or downregulated (lower panel) following ETS2 or chr21q22 editing within a ranked list of genes dysregulated following MEK1/2 inhibitor treatment (MEKi-treated inflammatory macrophages vs vehicle control). MEK1/2 inhibited using PD-0325901, 0.5 μM. Gene sets were derived from differential gene expression analysis (limma using voom transformation) following ETS2 disruption with gRNA1 (A), gRNA2 (B), or following chr21q22 deletion (C).

FIG. 18. Enrichment of macrophage signatures, derived from patients with the indicated diseases (colour coded by category), in ETS2-overexpressing macrophages (relative to control). Numbers represent Normalised enrichment score (NES), dashed line denotes FDR P 0.05.

FIG. 19. (A) Spatial transcriptomics of PSC and healthy liver (n=4). Images show representative fields of view with cell segmentation and semi-supervised clustering results (InsituType). Legend indicates InsituType cell-types. Hep., hepatocyte; LSECs, liver sinusoidal endothelial cells. (B) Average number of macrophages within a defined radius of a cholangiocyte. (C) Distance from cholangiocytes to nearest macrophage. Data shown as Tukey box-and-whisker plot. Mann-Whitney test, two-tailed. Data in (B) and (C) represent 10,532 PSC and 13,322 control cholangiocytes. (D) Scaled expression of ETS2-regulated genes in 21,067 PSC macrophages (excluding genes used to defined macrophage subsets) at defined distances from cholangiocytes. Data represent mean and 95% CI.

### DETAILED DESCRIPTION OF THE INVENTION

Below are provided certain definitions of terms, technical means, and embodiments used herein.

As used herein, the term “administration” refers to the administration of a composition to a subject. Administration to an animal subject (e.g., to a human) may be by any appropriate route. For example, in some embodiments, administration may be bronchial (including by bronchial instillation), buccal, enteral, intra-arterial, intra-dermal, intra-gastric, intra-medullary, intra-muscular, intra-nasal, intra-peritoneal, intra-thecal, intra-venous, intra-ventricular, within a specific organ or tissue (e. g. intra-hepatic, intra-tumoral, peri-tumoral, etc), mucosal, nasal, oral, rectal, subcutaneous, sublingual, topical, tracheal (including by intra-tracheal instillation), transdermal, vaginal and vitreal. The administration may involve intermittent dosing. Alternatively, administration may involve continuous dosing (e.g., perfusion) for at least a selected period of time.

As used herein, the term “Erythroblast Transformation Specific proto-oncogene 2” or “ETS2” refers to an amino acid sequence encoded by the ETS2 gene (HGNC ref 3489). The gene is also known as ETS2IT1. The human gene is encoded by the genomic sequence identified by Ensembl reference ENSG00000157557. Transcripts ENST00000360938.8, ENST00000360214.8 and ENST00000667466.1 can also be used to define the ETS2 sequence. In some embodiments, ETS2 may comprise an amino acid sequence of SEQ ID NO:4 (Uniprot reference: P15036). In some embodiments, ETS2 may consist of an amino acid sequence of SEQ ID NO:4 (Uniprot reference: P15036). In some embodiments, ETS2 may be encoded by an mRNA sequence comprising a sequence selected from any one of SEQ ID NO:2 and SEQ ID NO:3 (NCBI refs: NM_005239.6 and NM_001256295.2). In some embodiments, ETS2 may be encoded by an mRNA sequence consisting of a sequence selected from any one of SEQ ID NO:2 and SEQ ID NO:3 (NCBI refs: NM_005239.6 and NM_001256295.2). In some embodiments, ETS2 may be encoded by an mRNA sequence comprising the sequence of SEQ ID NO:2 NCBI ref: NM_005239.6). In some embodiments, ETS2 may be encoded by an mRNA sequence consisting of the sequence of SEQ ID NO:2 (NCBI ref: NM_005239.6).

As used herein, the term “Erythroblast Transformation Specific proto-oncogene 2 inhibitor” or “ETS2 inhibitor” refers to any agent that reduces expression or activity of an Erythroblast Transformation Specific proto-oncogene 2 (ETS2) polypeptide or protein. The reduction in expression or activity can be measured by any standard means known in the art, including measuring a reduction in the level of ETS2-encoding polynucleotide (such as ETS2 mRNA) and/or ETS2 polypeptide or protein. Methods for quantifying the amount of one or more ETS2 polynucleotides include fluorescent in situ hybridization (FISH), spectroscopy, quantitative sequencing, quantitative PCR (qPCR), RT-PCR, ddPCR, Southern blotting or northern blotting. Methods for quantifying the amount of one or more ETS2 polypeptides or protein include immunohistochemistry (IHC), spectroscopy, colorimetric detection, Western blotting, mass spectrometry or ELISA. The level of ETS2 inhibition provided by an ETS2 inhibitor may be at least 10%, at least 20%, at least 30%, at least 40%, at least 50%, at least 60%, at least 70%, at least 80%, at least 90%, at least 95% or at least 99% of the expression or activity of ETS2 when measured in the absence of the ETS2 inhibitor. Ideally, levels of ETS2 activity can be compared in the same model system (e.g. cell line or tissue) in the presence and in the absence of the ETS2 inhibitor.

As used herein, the term “inhibitor of the chr21q22 enhancer” or “chr21q22 enhancer inhibitor” refers to any agent that reduces expression or activity of an Erythroblast Transformation Specific proto-oncogene 2 (ETS2) polypeptide or protein via binding to the chr21q22 enhancer (SEQ ID NO:1). The reduction in expression or activity of ETS2 can be measured by any standard means known in the art, including measuring a reduction in the level of ETS2-encoding polynucleotide (such as ETS2 mRNA) and/or ETS2 polypeptide or protein. Methods for quantifying the amount of one or more ETS2 polynucleotides include fluorescent in situ hybridization (FISH), spectroscopy, quantitative sequencing, quantitative PCR (qPCR), RT-PCR, ddPCR, Southern blotting or northern blotting. Methods for quantifying the amount of one or more ETS2 polypeptides or protein include immunohistochemistry (IHC), spectroscopy, colorimetric detection, Western blotting, mass spectrometry or ELISA. The level of ETS2 inhibition provided by a chr21q22 enhancer inhibitor may be at least 10%, at least 20%, at least 30%, at least 40%, at least 50%, at least 60%, at least 70%, at least 80%, at least 90%, at least 95% or at least 99% of the expression or activity of ETS2 when measured in the absence of the chr21q22 enhancer inhibitor. Ideally, levels of ETS2 activity can be compared in the same model system (e.g. cell line or tissue) in the presence and in the absence of the chr21q22 enhancer inhibitor.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%), or less in either direction (greater than or less than) of the stated reference value unless otherwise stated or otherwise evident from the context (except where such number would exceed 100% of a possible value).

As used herein, the term “prevention” means delaying or forestalling the onset, development or progression of a disease, disorder, or condition for a period of time from minutes to indefinitely. “Prevention” also includes reducing the risk of developing a disease, disorder, or condition. “Prevention” includes, but does not require, complete avoidance of a disease condition.

As used herein, the term “treatment” (also “treat” or “treating”) refers to any administration of a therapeutic agent according to a therapeutic regimen that achieves a desired effect in that it partially or completely alleviates, ameliorates, relieves, reduces severity of and/or reduces incidence of one or more symptoms or features of a particular disease, disorder, and/or condition. In some embodiments, administration of the therapeutic agent according to the therapeutic regimen is correlated with achievement of the desired effect. Such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “comparable”, as used herein, refers to a system, set of conditions, effects, or results that is/are sufficiently similar to a test system, set of conditions, effects, or results, to permit scientifically legitimate comparison. Those of ordinary skill in the art will appreciate and understand which systems, sets of conditions, effects, or results are sufficiently similar to be “comparable” to any particular test system, set of conditions, effects, or results as described herein.

The term “correlates”, as used herein, has its ordinary meaning of “showing a correlation with”. Those of ordinary skill in the art will appreciate that two features, items or values show a correlation with one another if they show a tendency to appear and/or to vary, together. In some embodiments, a correlation is statistically significant when its p-value is less than 0.05; in some embodiments, a correlation is statistically significant when its p-value is less than 0.01. In some embodiments, correlation is assessed by regression analysis. In some embodiments, a correlation is a correlation coefficient.

As used herein, the terms “improve,” “increase” or “reduce,” or grammatical equivalents, indicate values that are relative to a reference (e.g., baseline) measurement, such as a measurement taken under comparable conditions (e.g., in the same individual prior to initiation of treatment described herein, or a measurement in a control individual (or multiple control individuals) in the absence of treatment) described herein.

As used herein, a “polypeptide” is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides may optionally include “non-natural” amino acids or other entities that nonetheless are capable of integrating into a polypeptide chain.

As used herein, the term “protein” refers to a molecule comprising a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

As used herein, the term “subject”, “individual”, or “patient” refers to any organism upon which embodiments of the invention may be used or administered, e.g., for experimental, diagnostic, prophylactic, and/or therapeutic purposes. Typical subjects include animals (e.g., mammals such as mice, rats, rabbits, non-human primates, and humans; insects; worms; etc.). In a preferred embodiment of the invention the subject is a human.

As used herein, the term “therapeutic regimen” refers to any method used to partially or completely alleviate, ameliorate, relieve, inhibit, prevent, delay onset of, reduce severity of and/or reduce incidence of one or more symptoms or features of a particular disease, disorder, and/or condition. It may include administration of one or more doses, optionally spaced apart by regular or varied time intervals. In some embodiments, a therapeutic regimen is one whose performance is designed to achieve and/or is correlated with achievement of (e.g., across a relevant population of cells, tissues, or organisms) a particular effect, e.g., reduction or elimination of a detrimental condition or disease. In some embodiments, treatment includes administration of one or more therapeutic agents either simultaneously, sequentially or at different times, for the same or different amounts of time. In some embodiments, a “treatment regimen” includes genetic methods such as gene therapy, gene ablation or other methods known to induce or reduce expression (e.g. transcription, processing, and/or translation of a particular gene product, such as a primary transcript or mRNA).

As used herein, the term “therapeutically effective amount” refers to an amount of a therapeutic agent which confers a therapeutic effect on the treated subject, at a reasonable benefit/risk ratio applicable to any medical treatment. Such a therapeutic effect may be objective (i.e., measurable by some test or marker) or subjective (i.e., subject gives an indication of or feels an effect). In some embodiments, “therapeutically effective amount” refers to an amount of a therapeutic agent or composition effective to treat, ameliorate, or prevent (e.g., delay onset of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, or on combination with other therapeutic agents. Alternatively or additionally, a specific therapeutically effective amount (and/or unit dose) for any particular patient may depend upon a variety of factors including the activity of the specific therapeutic agent employed; the specific composition employed; the age, body weight, general health, sex and diet of the patient; the time of administration, route of administration, and/or rate of excretion or metabolism of the specific therapeutic agent employed; the duration of the treatment; and like factors as is well known in the medical arts.

As used herein “portion” means a defined number of contiguous (i.e., linked) nucleobases of a nucleic acid. In some embodiments, a portion is a defined number of contiguous nucleobases of a target nucleic acid. In some embodiments, a portion is a defined number of contiguous nucleobases of an antisense compound. In some embodiments a portion may refer to 1, 2, 3, 4, 5, 6, 7, 8, 9, 10 or more contiguous nucleobases. In some embodiments a portion may refer to at least 10, at least 20, at least 30, at least 40 or at least 50 contiguous nucleobases. In some embodiments a portion may refer to fewer than 50, for example fewer than 40, fewer than 30, fewer than 20 or fewer than 10 contiguous nucleobases.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

As used herein, “nucleotide” means a nucleoside further comprising a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e. no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase. As used herein, “modified nucleoside” means a nucleoside comprising at least one chemical modification compared to naturally occurring RNA or DNA nucleosides. Modified nucleosides can comprise a modified sugar moiety and/or a modified nucleobase.

As used herein, “oligonucleotide” means a compound comprising a plurality of linked nucleosides. In some embodiments, an oligonucleotide comprises one or more unmodified ribonucleosides (RNA) and/or unmodified deoxyribonucleosides (DNA) and/or one or more modified nucleosides.

As used herein, “conjugate” or “conjugate group” means an atom, group of atoms or carrier bound to an inhibitory agent of the invention. In general, conjugate groups can modify one or more properties of the agent to which they are attached, including, but not limited to pharmacodynamic, pharmacokinetic, binding, absorption, cellular distribution, cellular uptake, charge and/or clearance properties.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In both DNA and RNA, guanine (G) is complementary to cytosine (C). In some embodiments, complementary nucleobase means a nucleobase of an oligomeric compound that is capable of base pairing with a nucleobase of its target sequence. For example, if a nucleobase at a certain position of an oligomeric compound is capable of hydrogen bonding with a nucleobase at a certain position of a target sequence, then the position of hydrogen bonding between the oligomeric compound and the target sequence is considered to be complementary at that nucleobase pair. Nucleobases comprising certain modifications may maintain the ability to pair with a counterpart nucleobase and thus, are still capable of nucleobase complementarity.

As used herein, “fully complementary” in reference to an oligomeric compound or region thereof means that each nucleobase of the oligomeric compound or region thereof is capable of pairing with a nucleobase of a complementary nucleic acid target sequence or a self-complementary region of the oligomeric compound. Thus, a fully complementary oligomeric compound or region thereof comprises no mismatches or unhybridized nucleobases with respect to its target sequence or a self-complementary region of the oligomeric compound.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression.

As used herein, “activates” or “protein that activates” refers to the process of turning on or up-regulating the expression of a gene, resulting in increased production and/or activity of its corresponding protein or RNA product. This process is controlled by various molecular mechanisms, including transcription factors, epigenetic modifications, and signaling pathways. In genetic activation, specific regulatory sequences within the DNA sequence, such as promoters and enhancers, are recognized and bound by transcription factors, which recruit RNA polymerase to initiate transcription of the gene. This results in the production of messenger RNA (mRNA), which is then translated into protein by the ribosomes. Genetic activation can be influenced by various factors, such as environmental cues, developmental stages, and physiological conditions.

As used herein, “PROTAC” (PROteolysis TArgeting Chimeras) refers to a type of small molecule therapeutic agent that harnesses the ubiquitin-proteasome system to selectively degrade target proteins within cells. A PROTAC molecule consists of two functional domains: a ligand that binds to the target protein, and a ligand that recruits an E3 ubiquitin ligase enzyme. Once the PROTAC molecule binds to the target protein and the E3 ubiquitin ligase, it brings them into close proximity, resulting in the transfer of ubiquitin molecules to the target protein. Ubiquitinated proteins are then recognized and degraded by the proteasome, leading to a reduction in the levels of the target protein. PROTACs have several potential advantages over traditional small molecule inhibitors, including the ability to target previously undruggable proteins, greater selectivity, and the potential for longer-lasting effects. They have shown promise as a therapeutic strategy for a wide range of diseases, including cancer, neurodegenerative disorders, and viral infections.

As used herein, “molecular glue” refers to a class of small molecules that can enhance protein-protein interactions within cells. These molecules act as “glue” by binding simultaneously to two or more protein surfaces, thereby stabilizing interactions that would otherwise be transient or weak. In the context of drug discovery, molecular glue molecules can be used to create new therapeutic strategies by targeting disease-causing proteins that were previously considered “undruggable” with traditional small molecules. By stabilizing protein-protein interactions that are essential for disease pathogenesis, molecular glue molecules can inhibit or enhance protein function in ways that cannot be achieved with traditional small molecule inhibitors. Molecular glue has shown promise as a therapeutic approach for a range of diseases, including cancer, neurodegenerative disorders, and viral infections. However, there are still significant challenges to overcome in the design and development of effective molecular glue molecules, including issues with selectivity and off-target effects.

As used herein, “agent that disrupts the function of co-transcriptional activators” refers to a molecule or compound that interferes with the activity of proteins or complexes involved in the process of co-transcriptional activation of gene expression. Co-transcriptional activators are proteins or complexes that interact with RNA polymerase during transcription, facilitating the recruitment and binding of other proteins necessary for proper gene expression. Disrupting the function of co-transcriptional activators can affect the ability of the cell to produce functional RNA molecules, leading to changes in gene expression and potentially contributing to disease. Some examples of co-transcriptional activators include chromatin remodelers, histone acetyltransferases, and transcription elongation factors. Agents that disrupt the function of co-transcriptional activators can act in various ways, such as inhibiting their enzymatic activity, preventing their binding to other proteins or RNA molecules, or inducing their degradation. These agents can be used as potential therapeutics to treat diseases that are caused by dysregulation of gene expression.

As used herein, “agent that targets chromatin regulators” refers to a molecule or compound that interacts with proteins involved in the modification, remodeling, or packaging of chromatin, the complex of DNA and proteins that makes up chromosomes within the nucleus of a cell. Chromatin regulators play crucial roles in controlling gene expression by modulating the accessibility of DNA to transcription factors and other regulatory proteins. Agents that target chromatin regulators can act in various ways, such as inhibiting their enzymatic activity, preventing their binding to other proteins or DNA, or inducing their degradation. These agents can be used as potential therapeutics to treat diseases that are caused by dysregulation of gene expression. Examples of chromatin regulators that can be targeted by such agents include histone deacetylases (HDACs), histone methyltransferases (HMTs), histone demethylases (HDMs), and chromatin remodelers.

As used herein, “agent that targets non-coding RNA” refers to a molecule or compound that interacts with RNA molecules that do not code for proteins, also known as non-coding RNA (ncRNA). Non-coding RNA molecules play crucial roles in regulating gene expression, cell signaling, and other cellular processes, and are increasingly recognized as important therapeutic targets for a wide range of diseases. Agents that target non-coding RNA can act in various ways, such as blocking their interaction with other proteins or RNA molecules, promoting their degradation, or modulating their function. These agents can be used as potential therapeutics to treat diseases that are caused by dysregulation of non-coding RNA. Examples of non-coding RNA molecules that can be targeted by such agents include microRNAs (miRNAs), long non-coding RNAs (lncRNAs), and circular RNAs (circRNAs).

As used herein, “GlyPro” or “GlyPro linker” refers to a type of flexible amino acid linker that is commonly used in the design of protein therapeutics, particularly antibody-drug conjugates (ADCs). The GlyPro linker is composed of glycine and proline residues in a repeating pattern, which provides flexibility to the linker while minimizing the potential for immunogenicity and other adverse effects. In the context of ADCs, the GlyPro linker is used to connect the antibody portion of the molecule to a cytotoxic drug or other therapeutic payload. The linker plays a crucial role in determining the pharmacokinetics and pharmacodynamics of the ADC, including its stability, solubility, and ability to target specific cells or tissues. GlyPro is selectively cleaved by β-glucuronidase, thereby facilitating selective release in lysosomes. Additionally, this linker has advantageous hydrophilic properties, which should reduce aggregation and promote solubility. Exemplary GlyPro linkers and related methods can be found in US2008241128, which is incorporated herein by reference in its entirety.

As used herein, “esterase-sensitive motif” refers to a molecular structure or sequence that is cleaved by esterases, a class of enzymes that hydrolyze ester bonds. In the context of drug delivery or controlled release, an esterase-sensitive motif can be used to create a prodrug or polymer-based system that is activated by esterases in a specific tissue or cell type. One common example of an esterase-sensitive motif is a carboxylic acid ester bond, which can be hydrolyzed by esterases to release the active drug molecule. In a prodrug system, the drug molecule is chemically linked to a carrier molecule via an esterase-sensitive motif, which allows for targeted delivery and activation of the drug. In a polymer-based system, the polymer backbone contains esterase-sensitive motifs that allow for controlled release of the encapsulated drug in response to esterase activity. Esterase-sensitive motifs have been used in a variety of drug delivery and controlled release systems, including micelles, liposomes, and nanoparticles. They offer several advantages over other types of activation mechanisms, including high specificity, rapid kinetics, and compatibility with a wide range of drugs and polymers. Unlike most human carboxylesterases, which are ubiquitously expressed, human carboxylesterase-1 (hCE-1) is predominantly expressed in monocyte-derived cells (e.g. monocytes and macrophages). ESM technology uses esters that are selectively hydrolysed by hCE-1, resulting in intracellular accumulation of the pharmacologically active acid (drug) in hCE-1-expressing cells because, unlike the ester, the acid cannot readily diffuse out. This approach can facilitate a 1000-fold increase in potency over a non-conjugated inhibitor. Exemplary esterase-sensitive motifs and related methods can be found in WO06117567, which is incorporated herein by reference in its entirety.

As used herein, “Crohn's disease” refers to a chronic inflammatory bowel disease (IBD) that affects the gastrointestinal tract. It can occur anywhere along the digestive tract, from the mouth to the anus, but most commonly affects the small intestine and the beginning of the large intestine (colon). The exact cause of Crohn's disease is unknown, but it is thought to be an autoimmune condition, where the body's immune system attacks the healthy tissues of the digestive tract. Genetics, environmental factors, and an abnormal immune response may all play a role in the development of the disease. The symptoms of Crohn's disease can vary widely and include abdominal pain, diarrhea, rectal bleeding, weight loss, and fatigue. Other complications may arise, including malnutrition, intestinal obstruction, and abscesses.

As used herein, ulcerative colitis” refers to a chronic inflammatory bowel disease (IBD) that affects the colon (large intestine) and rectum. It is characterized by inflammation and ulcers (sores) that develop on the lining of the colon, which can lead to symptoms such as diarrhea, rectal bleeding, abdominal pain, and weight loss. The exact cause of ulcerative colitis is not known, but it is believed to be an autoimmune disorder, in which the immune system mistakenly attacks the healthy tissues of the colon and rectum. Genetics and environmental factors may also play a role in the development of the disease. Ulcerative colitis is typically diagnosed through a combination of physical exams, medical history, blood tests, stool tests, and colonoscopy (an examination of the colon with a flexible tube). Treatment for ulcerative colitis depends on the severity of the disease and may include medications to control inflammation, antibiotics to treat infections, and surgery to remove the affected portions of the colon and rectum. Lifestyle changes, such as dietary modifications, stress management, and regular exercise, may also help manage symptoms and improve quality of life.

As used herein, “ankylosing spondylitis” (AS) refers to a type of chronic inflammatory arthritis that primarily affects the spine, but can also involve other joints in the body. It is a form of spondyloarthritis, a group of inflammatory diseases that cause arthritis and inflammation in the spine and other joints. AS causes inflammation of the joints between the vertebrae of the spine, which can result in pain and stiffness in the lower back and buttocks. Over time, the inflammation can lead to the fusion of the vertebrae, which can cause the spine to become rigid and inflexible. AS can also affect other joints in the body, such as the hips, knees, and shoulders. The exact cause of AS is unknown, but it is believed to be an autoimmune disorder, in which the immune system mistakenly attacks the healthy tissues of the body. Genetics and environmental factors may also play a role in the development of the disease. Diagnosis of AS involves a combination of physical exams, medical history, blood tests, imaging studies (such as X-rays and MRI), and assessment of symptoms. Treatment for AS may include medications to reduce inflammation and pain, physical therapy to maintain mobility and flexibility, and lifestyle modifications, such as regular exercise and proper posture. In severe cases, surgery may be required to correct deformities or replace damaged joints.

As used herein “primary sclerosing cholangitis” (PSC) refers to a chronic liver disease that affects the bile ducts, which are the tubes that carry bile from the liver to the small intestine. It is characterized by inflammation and scarring of the bile ducts, which can lead to obstruction and damage to the liver. The exact cause of PSC is unknown, but it is believed to be an autoimmune disorder, in which the immune system mistakenly attacks the bile ducts. Genetics and environmental factors may also play a role in the development of the disease. Symptoms of PSC can include fatigue, itching, jaundice (yellowing of the skin and eyes), abdominal pain, and weight loss. Over time, PSC can lead to complications such as cirrhosis (scarring of the liver), liver failure, and an increased risk of liver cancer. Diagnosis of PSC typically involves blood tests, imaging studies (such as ultrasound, MRI, or CT scans), and a procedure called ERCP (endoscopic retrograde cholangiopancreatography), which uses a flexible tube with a camera to examine the bile ducts. Treatment for PSC involves managing symptoms and preventing complications. Medications may be used to reduce inflammation and control symptoms such as itching. In severe cases, liver transplant may be necessary. Regular monitoring and follow-up with a healthcare provider is important for people with PSC.

As used herein, “Takayasu arteritis” (TA) refers to a rare type of chronic inflammatory disease that affects the large arteries in the body, particularly the aorta and its branches. It is also known as “pulseless disease” because it can cause a weak or absent pulse in the arms and legs. TA is believed to be an autoimmune disorder, in which the immune system mistakenly attacks the healthy tissues of the arteries, causing inflammation and damage. The exact cause of TA is not fully understood, but genetics and environmental factors may also play a role in the development of the disease. Symptoms of TA can include fatigue, fever, weight loss, muscle pain, and joint pain. As the disease progresses, it can cause narrowing or blockages in the arteries, which can lead to a variety of complications, such as high blood pressure, heart failure, stroke, and aneurysms (weak areas in the artery walls that can rupture). Diagnosis of TA involves a combination of physical exams, medical history, blood tests, imaging studies (such as MRI or CT scans), and assessment of symptoms. Treatment for TA involves medications to reduce inflammation and prevent further damage to the arteries. In some cases, surgery may be necessary to repair or bypass blocked arteries.

As used herein, “rheumatoid arthritis” (RA) is a chronic autoimmune disorder that primarily affects the joints. It is characterized by inflammation of the synovial membrane, which is the lining of the joint capsule, leading to joint pain, swelling, stiffness, and damage. RA can affect any joint in the body, but is most commonly seen in the hands, wrists, and feet. In addition to joint symptoms, RA can also cause fatigue, fever, and a general feeling of malaise. The exact cause of R^{A} is not known, but it is believed to involve a combination of genetic and environmental factors that trigger the immune system to attack the synovial membrane. Smoking and obesity are two examples of environmental factors that have been associated with an increased risk of developing RA. Diagnosis of RA involves a combination of physical exams, medical history, blood tests (including tests for rheumatoid factor and anti-CCP antibodies), and imaging studies (such as X-rays and MRI). Early diagnosis and treatment of RA is important in order to prevent joint damage and improve long-term outcomes.

As used herein “Reactive oxygen species” (ROS) refers to chemically reactive molecules containing oxygen that are produced as byproducts of normal cellular metabolism. They include superoxide anion, hydrogen peroxide, and hydroxyl radical, among others. ROS are important signaling molecules involved in many cellular processes, including cell proliferation, apoptosis, and inflammation. However, excessive levels of ROS can be harmful to cells and tissues, as they can cause oxidative damage to DNA, proteins, and lipids. This damage can lead to mutations, cellular dysfunction, and cell death, and has been implicated in the development of a variety of diseases, including cancer, neurodegenerative diseases, and cardiovascular disease. The body has several mechanisms to neutralize ROS, including antioxidant enzymes such as superoxide dismutase, catalase, and glutathione peroxidase, as well as small molecules such as vitamin C, vitamin E, and glutathione. However, when these defence mechanisms are overwhelmed, oxidative stress can occur, leading to cell and tissue damage.

As used herein, “macrophage phagocytosis” refers to the process by which macrophages, a type of immune cell, engulf and internalize foreign particles, such as bacteria, viruses, and debris from dead cells. Phagocytosis is a critical mechanism of the innate immune response, allowing macrophages to identify and remove pathogens and other harmful substances from the body. The process of phagocytosis involves several steps. First, macrophages use receptors on their cell surface to recognize and bind to the foreign particle. Next, the macrophage extends pseudopodia (projections of the cell membrane) around the particle, forming a phagosome. The phagosome then fuses with lysosomes, which contain enzymes that break down the contents of the phagosome. The resulting material is then either released from the macrophage or presented to other immune cells for further processing.

As used herein, “macrophage migration” refers to the process by which macrophages, a type of immune cell, move from one location to another within the body. This movement is essential for macrophages to perform their functions in immune surveillance, tissue repair, and inflammation. Macrophage migration occurs in response to various stimuli, including chemokines, growth factors, and microbial products. These stimuli activate signaling pathways within the macrophage that induce changes in cytoskeletal organization and cell polarity, allowing the macrophage to move towards the source of the stimulus. Macrophage migration is a complex process that involves several steps, including adhesion to the extracellular matrix, polarization of the cell, and extension of pseudopodia to allow movement. During migration, macrophages can interact with other immune cells, such as T cells and B cells, as well as with other cell types in the tissue microenvironment, including fibroblasts and endothelial cells.

As used herein, macrophage activation refers to the process by which macrophages, a type of immune cell, are activated in response to a stimulus such as infection or tissue damage. Activated macrophages undergo a series of changes in gene expression, morphology, and function that allow them to carry out their role in the immune response more effectively. Macrophage activation can occur through two different pathways: classical activation and alternative activation. Classical activation, also known as M1 activation, occurs in response to pro-inflammatory signals such as interferon-gamma (IFN-γ) and lipopolysaccharide (LPS), which are produced during infection or inflammation. M1 macrophages are characterized by increased production of pro-inflammatory cytokines, enhanced phagocytic activity, and increased expression of surface molecules involved in antigen presentation and T cell activation. Alternative activation, also known as M2 activation, occurs in response to anti-inflammatory signals such as interleukin-4 (IL-4) and interleukin-13 (IL-13), which are produced during tissue repair and wound healing. M2 macrophages are characterized by decreased production of pro-inflammatory cytokines and increased production of anti-inflammatory cytokines and growth factors. They also have enhanced phagocytic activity for apoptotic cells and debris, and play a role in tissue remodeling and angiogenesis.

As used herein, “proinflammatory cytokines” refer to a group of signaling proteins that are involved in the inflammatory response of the immune system. These cytokines are typically produced by immune cells such as macrophages, T cells, and mast cells, in response to various stimuli such as infection, injury, or stress. Examples of proinflammatory cytokines include interleukin-1 (IL-1), interleukin-6 (IL-6), tumor necrosis factor alpha (TNF-α), interferon-gamma (IFN-γ), and interleukin-17 (IL-17), granulocyte-macrophage colony-stimulating factor (GM-CSF) or other interleukins such as IL-8, IL-12, IL-18 and IL-23. These cytokines can activate immune cells and cause inflammation, which is characterized by redness, swelling, heat, and pain. Proinflammatory cytokines play a crucial role in the immune response to infection and tissue damage. They help to recruit immune cells to the site of infection or injury, promote the destruction of pathogens, and initiate tissue repair. However, excessive or prolonged production of proinflammatory cytokines can lead to chronic inflammation, tissue damage, and autoimmune diseases.

As used herein, the term “specifically binds” means that the antibody recognizes and binds to a specific target molecule, such as a protein or a small molecule, with high affinity and selectivity. This is also known as immunospecific binding. The specificity of an antibody is determined by the unique three-dimensional structure of its variable regions, which form the antigen-binding site that interacts with the target molecule. The specificity of an antibody for its target molecule is usually determined experimentally, for example, by measuring its binding affinity or by testing its ability to selectively recognize the target molecule in a complex mixture of other molecules. Binding specificity or immunospecific binding of antibodies may be assayed for by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays or protein A immunoassays.

As used herein, the term “antibody-drug ratio” (ADR) is a term used in the context of antibody-drug conjugates (ADCs), which are a type of targeted therapy that combines the specificity of an antibody with the cytotoxicity of a drug. The ADR refers to the number of drug molecules that are conjugated to each antibody molecule in an ADC. It is typically expressed as a ratio, such as 2:1 or 4:1, indicating the number of drug molecules per antibody molecule. The ADR can vary depending on the design and synthesis of the ADC, and it is an important parameter that can affect the efficacy and safety of the therapy. The optimal ADR for an ADC depends on several factors, such as the potency of the drug, the binding affinity of the antibody, and the expression level of the target antigen. A higher ADR can increase the potency of the therapy by delivering more drug molecules to the target cells, but it can also increase the risk of toxicity by increasing the non-specific binding of the ADC to healthy cells. A lower ADR can decrease the risk of toxicity but may also reduce the efficacy of the therapy. The ADR is an important parameter that is evaluated during the preclinical and clinical development of ADCs to optimize their therapeutic properties.

As used herein, the term “chr21q22-associated disease” refers to diseases or disorders that have been linked to the intergenic region on chr21q22. In particular, chr21q22-associated diseases includes Crohn's disease, ulcerative colitis, primary sclerosing cholangitis, ankylosing spondylitis, and Takayasu arteritis.

The term “autoinflammatory disease” refers to diseases or disorders involving episodes of inflammation triggered by the innate immune system.

The term “comprising” is used herein to mean including the method steps or elements identified, but that such steps or elements do not comprise an exclusive list and as such there may be present additional steps or elements.

Further, to the extent that the term “includes” is used in either the detailed description or the claims, such term is intended to be inclusive in a manner similar to the term “comprising” as “comprising” is interpreted when employed as a transitional word in a claim.

### Pharmaceutical Compositions of the Agent

As used herein “pharmaceutical composition” means a mixture of substances suitable for administering to an individual. For example, a pharmaceutical composition may comprise one or more active pharmaceutical agents and a sterile aqueous solution.

As used herein “pharmaceutically acceptable salts” means physiologically and pharmaceutically acceptable salts of antisense compounds, i.e., salts that retain the desired biological activity of the parent oligonucleotide and do not impart undesired toxicological effects thereto.

Other aspects of the present invention also relate to a medicinal product or a diagnostic aid comprising an inhibitory agent according to the invention, a nucleic acid according to the invention or a cell according to the invention and, where appropriate, suitable excipients and additives, such as, for example, a physiological saline solution, stabilizers or proteinase inhibitors.

### Kits

Any pharmaceutical composition described herein can be provided in a kit. In some instances, the kit includes (a) a container that contains a pharmaceutical composition described herein and, optionally (b) informational material. The informational material can be descriptive, instructional, marketing or other material that relates to the methods described herein and/or the use of an agent, e.g., for therapeutic benefit.

The informational material of the kits is not limited in its form. In some instances, the informational material can include information about production of an agent of the invention, concentration, date of expiration, batch or production site information, and so forth. In other situations, the informational material relates to methods of administering an agent of the invention, e.g., in a suitable amount, manner, or mode of administration (e.g., a dose, dosage form, or mode of administration described herein).

In some cases, the informational material, e.g., instructions, is provided in printed matter, e.g., a printed text, drawing, and/or photograph, e.g., a label or printed sheet. The informational material can also be provided in other formats, such as Braille, computer readable material, video recording, or audio recording. In other instances, the informational material of the kit is contact information, e.g., a physical address, email address, website, or telephone number, where a user of the kit can obtain substantive information about a therapeutic agent therein and/or their use in the methods described herein. The informational material can also be provided in any combination of formats.

In addition to a pharmaceutical composition of the invention, the kit can include other ingredients, such as a solvent or buffer, a stabilizer, or a preservative. The kit can also include further agents, e.g., a second or third agent, e.g., other therapeutic agents or other therapeutic compounds or compositions. The components can be provided in any form, e.g., liquid, dried or lyophilized form. The components can be substantially pure (although they can be combined together or delivered separate from one another) and/or sterile. When the components are provided in a liquid solution, the liquid solution can be an aqueous solution, such as a sterile aqueous solution. When the components are provided as a dried form, reconstitution generally is by the addition of a suitable solvent. The solvent, e.g., sterile water or buffer, can optionally be provided in the kit.

The kit can include one or more containers for a pharmaceutical composition or other agents. In some cases, the kit contains separate containers, dividers or compartments for a therapeutic agent and informational material. For example, a therapeutic agent can be contained in a bottle, vial, or syringe, and the informational material can be contained in a plastic sleeve or packet. In other situations, the separate elements of the kit are contained within a single, undivided container. In some cases, the kit can include a plurality (e.g., a pack) of individual containers, each containing one or more unit dosage forms (e.g., a dosage form described herein) of an agent of the invention. The containers can include a unit dosage, e.g., a unit that includes a therapeutic agent. For example, the kit can include a plurality of syringes, ampules, foil packets, blister packs, or medical devices, e.g., each containing a unit dose. The containers of the kits can be air tight, waterproof (e.g., impermeable to changes in moisture or evaporation), and/or light-tight.

The kit can optionally include a device suitable for administration of an agent of the invention, e.g., a syringe or other suitable delivery device. The device can be provided preloaded with an agent of the invention, e.g., in a unit dose, or can be empty, but suitable for loading.

### CRISPR

As used herein, “CRISPR nuclease system” refers collectively to transcripts and other elements involved in the expression of or directing the activity of CRISPR-associated (“Cas”) genes, including sequences encoding a Cas gene, a guide sequence (also referred to as a “spacer” in the context of an endogenous CRISPR system), or other sequences and transcripts from a CRISPR locus.

In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. A CRISPR system is characterized by elements that promote the formation of a CRISPR complex at the site of a target sequence.

In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. in some embodiments, the target sequence may be within an organelle of a eukaryotic cell, for example, mitochondrion or chloroplast.

When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell. In some embodiments, a vector comprises a regulatory element operably linked to an enzyme-coding sequence encoding a CRISPR enzyme, such as a Cas protein, also called Cas enzyme.

Non-limiting examples of Cas proteins (or Cas enzymes) include Cas1, Cas1.13, Cas2, Cas3, Cas4, Cas5, Cas6, Cas7, Cas8, Cas9 (also known as Csn1 and Csx12), Cas10, Csy1, Csy2, Csy3, CseI, Cse2, Csc1, Csc2, Csa5, Csn2, Csm2, Csm3, Csm4, Csmr5, Csm6, Cmr1, Cmr3, Cmr4, Cmr5, Crnr6, CsbI, Csb2, Csb3, Csx17, Csx14, Csx10, Csx16, CsaX, Csx3, Csx1, Csx15, Csf1, Csf2, Csf3, Csf4, homologs thereof, or modified versions thereof.

These enzymes are known; for example, the amino acid sequence of S. pyogenes Cas9 protein may be found in the SwissProt database under accession number Q99ZW2. In some embodiments, the CRISPR enzyme has DNA cleavage activity, such as Cas9. In some embodiments the CRISPR enzyme is Cas9, and may be Cas9 from S. pyogenes or S. pneumoniae.

In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, the CRISPR enzyme directs cleavage of a nucleic acid sequence encoding ETS2 or a nucleic acid sequence encoding the enhancer region at chr21q22.

### Alternative Modalities

In some embodiments, the ETS2 or chr21q22 enhancer inhibitor comprises a small molecule. In some embodiments, the ETS2 or chr21q22 enhancer inhibitor comprises an inhibitory peptide. In some embodiments, the ETS2 or chr21q22 enhancer inhibitor is an inhibitory nucleic acid molecule. In some embodiments, the ETS2 or chr21q22 enhancer inhibitor comprises an antibody.

### Combination Therapies

In some embodiments, the invention features a composition (e.g., one or more compositions, formulations or dosage formulations) or a pharmaceutical combination, comprising an inhibitory agent according to the invention and a second therapeutic agent.

In some embodiments, the composition comprises a pharmaceutically acceptable carrier. In some embodiments, the inhibitory agent according to the invention and the second agent can be present in a single composition or as two or more different compositions. The inhibitory agent according to the invention and the second agent can be administered via the same administration route or via different administration routes. The inhibitory agent according to the invention and the second agent can be administered simultaneously or sequentially. In some embodiments, the pharmaceutical combination comprises the inhibitory agent according to the invention and the second agent separately or together.

### EXAMPLES

### Example 1—Resolving Molecular Mechanisms at the Pleiotropic Chr21q22 Locus

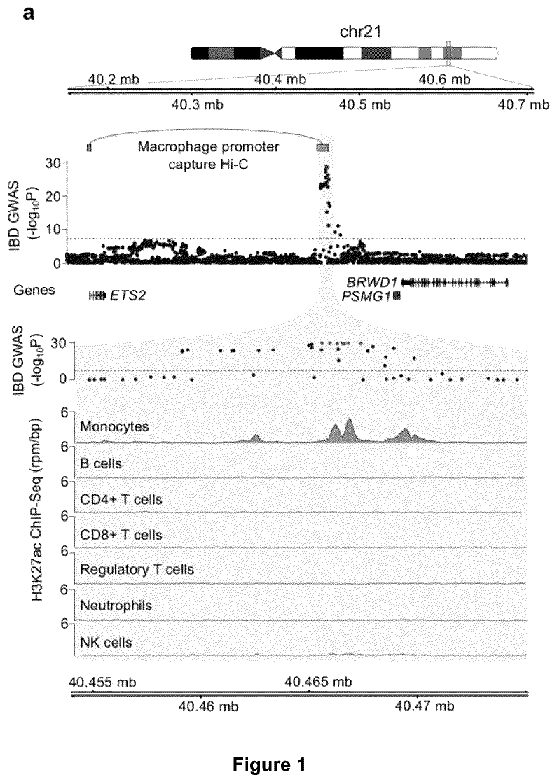

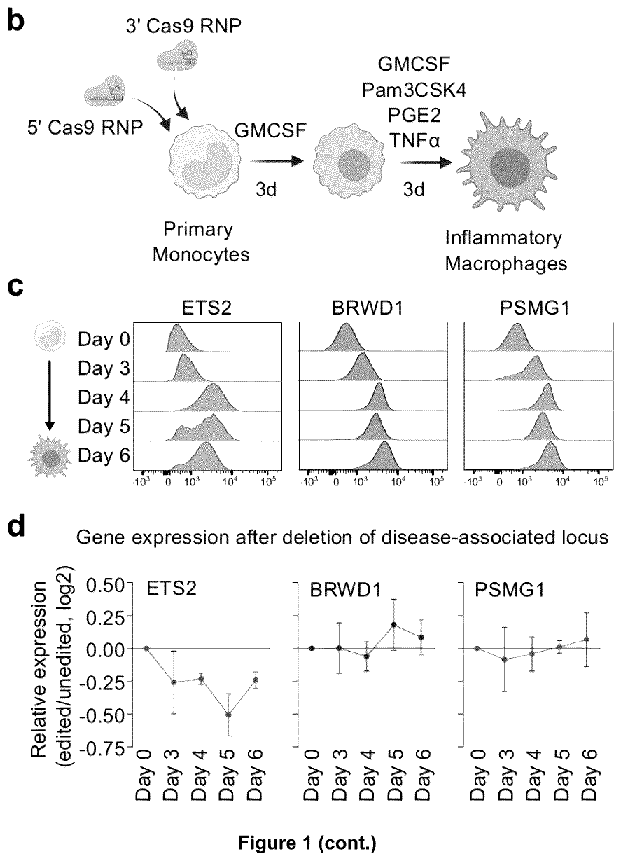

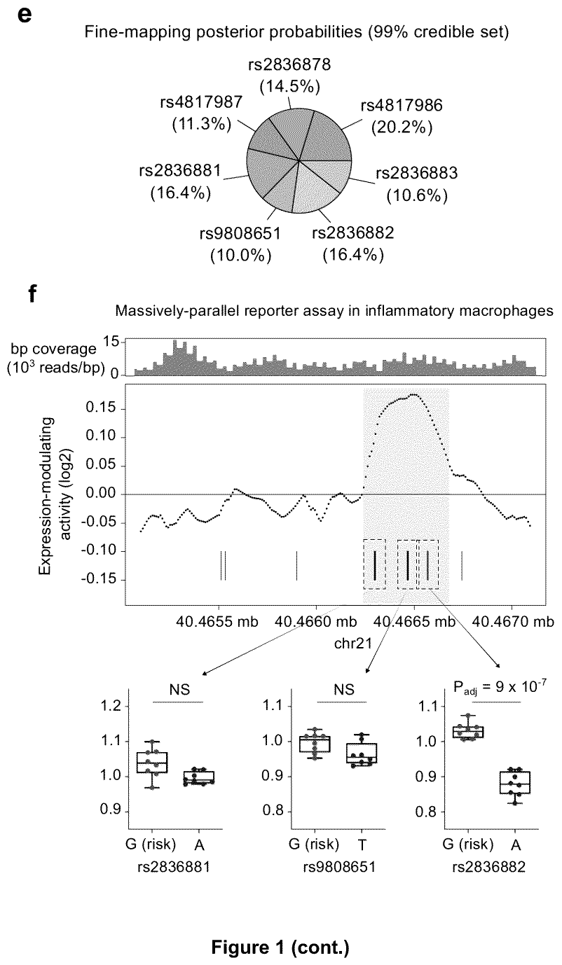

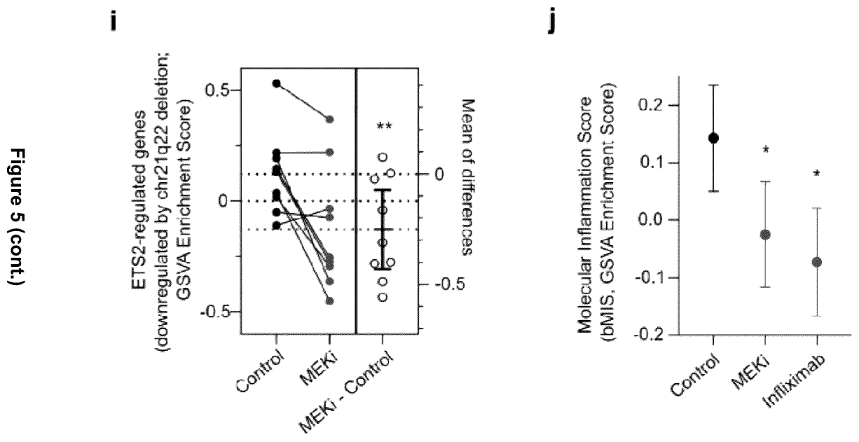

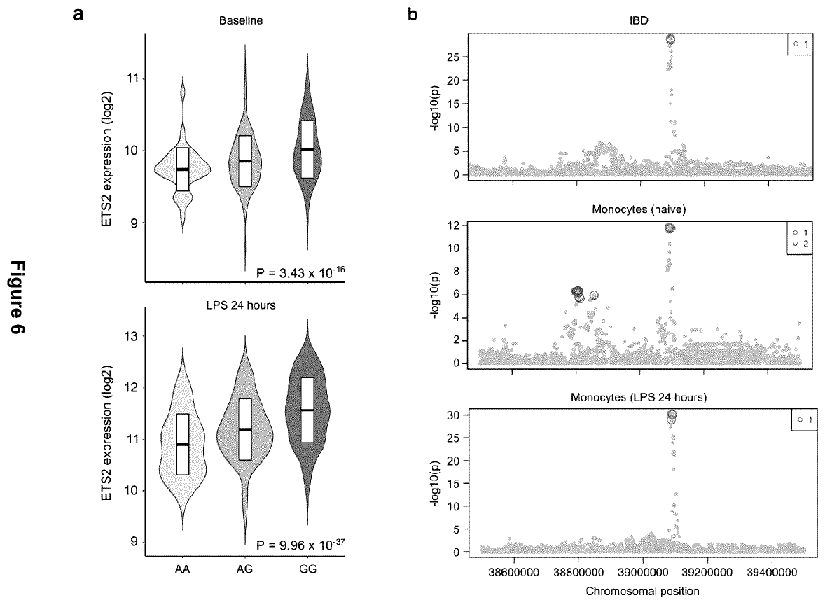

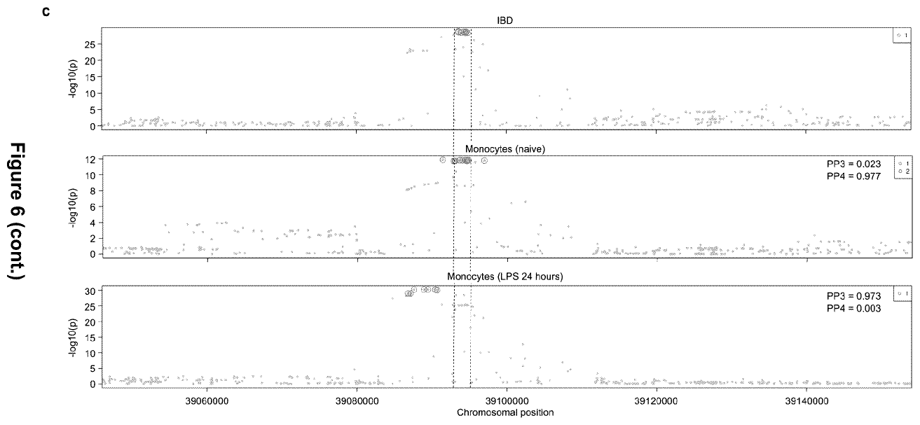

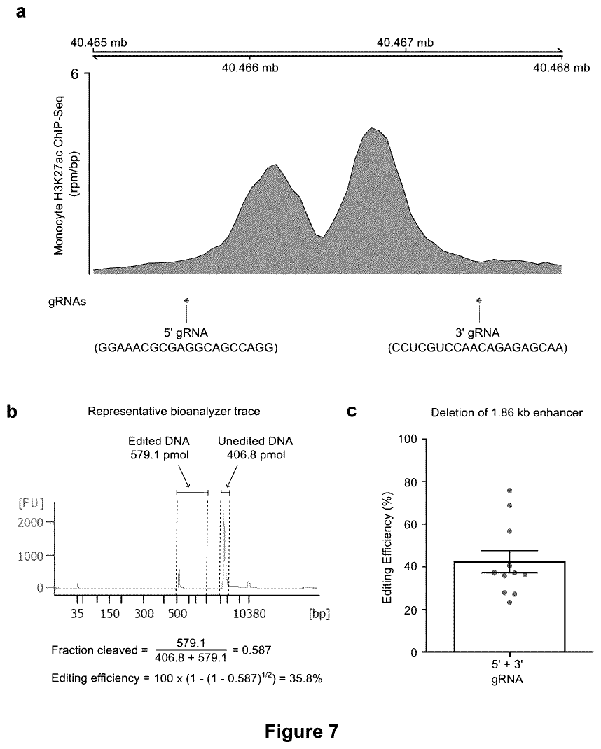

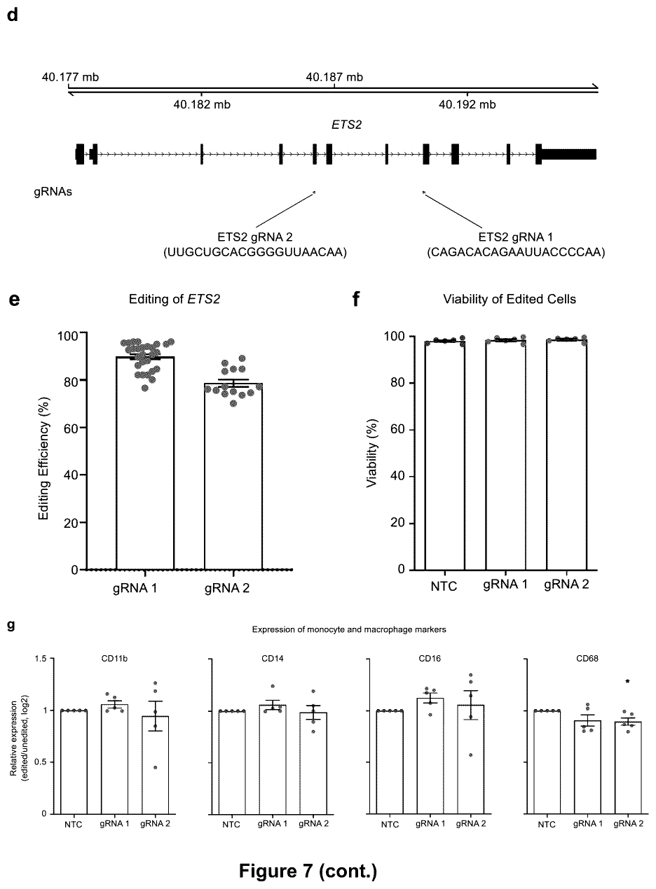

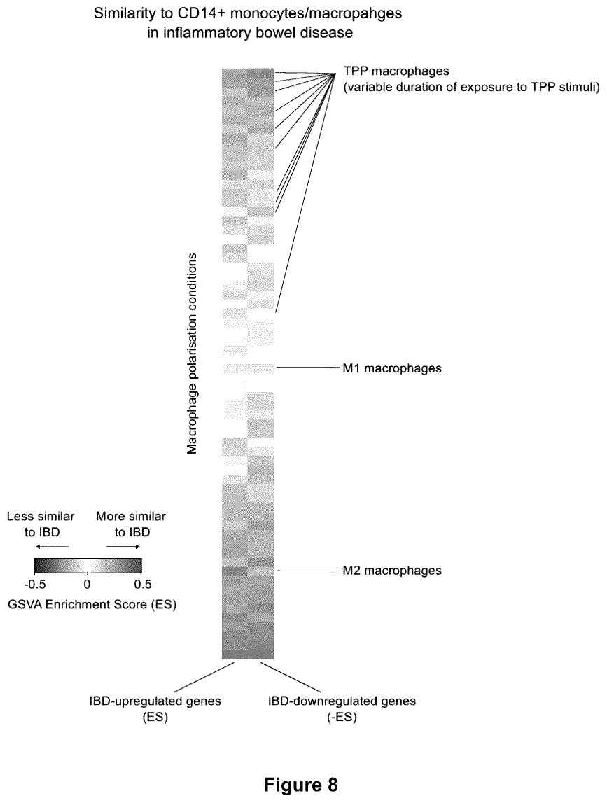

Several genetic variants predispose to more than one disease-highlighting both their biological importance and an opportunity to discover shared disease mechanisms. One notable example is an intergenic haplotype on chr21q22, which has been independently associated with Crohn's disease [7], ulcerative colitis [7] (collectively IBD), ankylosing spondylitis [8], primary sclerosing cholangitis [9], and Takayasu arteritis [10]. Although the associated locus does not contain any genes, there are several nearby candidates including PSMG1, BRWD1 and ETS2 (FIG. 1a), all of which have been nominated as being potentially causal in different studies [7,8,9,10,11]. The underlying biological mechanisms, however, remain unknown. We hypothesised that this intergenic locus must be a distal enhancer and—since the associated diseases are all immune-mediated despite affecting different organs—searched for features of enhancer activity in disease-relevant immune cell-types. Using H3K27ac ChIP-seq data, which marks active enhancers/promoters, we found that the chr21q22 locus contains a monocyte/macrophage-specific enhancer (FIG. 1a). Monocytes and monocyte-derived inflammatory macrophages play a central role in the pathogenesis of many autoimmune and inflammatory diseases, producing cytokines that are often targeted by the most effective therapies [12]. To identify the causal gene at this locus, we first considered publicly-available data, including promoter-capture Hi-C [13] and eQTL datasets from human monocytes. We found that the disease-associated locus physically interacts with the promoter of ETS2, located ~290-kb away, and that the risk haplotype correlates with higher ETS2 expression in monocytes, especially after activation (FIG. 6). Interestingly, however, the eQTL signal in activated monocytes did not statistically colocalise with the disease association, although colocalisation was observed with a weaker eQTL in resting monocytes (FIG. 6). To more directly confirm the identity of the target gene, we designed CRISPR guide (g) RNAs to flank the 1.85-kb enhancer region and delivered these as Cas9-gRNA ribonucleoprotein (RNP) complexes into primary human monocytes (FIG. 1b, FIG. 7). Deletion of the entire enhancer was achieved, on average, in 44% of cells (FIG. 7). The transfected cells were then cultured in the presence several inflammatory mediators, including TNFα (a proinflammatory cytokine), prostaglandin E2 (an inflammatory lipopeptide) and Pam3CSK4 (a TLR1/2 agonist). This model, termed “TPP”, was designed to mimic a chronic inflammatory environment [15], and better recapitulates the state of patient-derived monocytes/macrophages than classical M1 or M2 models ([16] and FIG. 7). Because flow cytometry antibodies were not available for any of the genes at chr21q22, we used PrimeFlow to measure the dynamics of RNA transcription following CRISPR editing of the disease-associated locus. In unedited cells, expression of all 3 candidate genes (ETS2, BRWD1, PSMG1) increased following exposure to inflammatory stimuli (FIG. 1c). Deletion of the chr21q22 enhancer did not affect the upregulation of BRWD1 and PSMG1, but expression of ETS2 was significantly reduced (FIG. 1d)—confirming that this pleiotropic locus is a monocyte/macrophage-specific distal enhancer of ETS2.

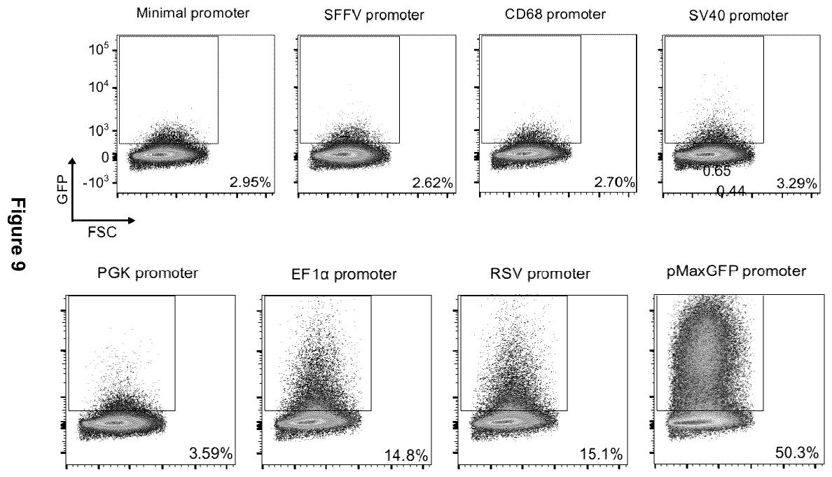

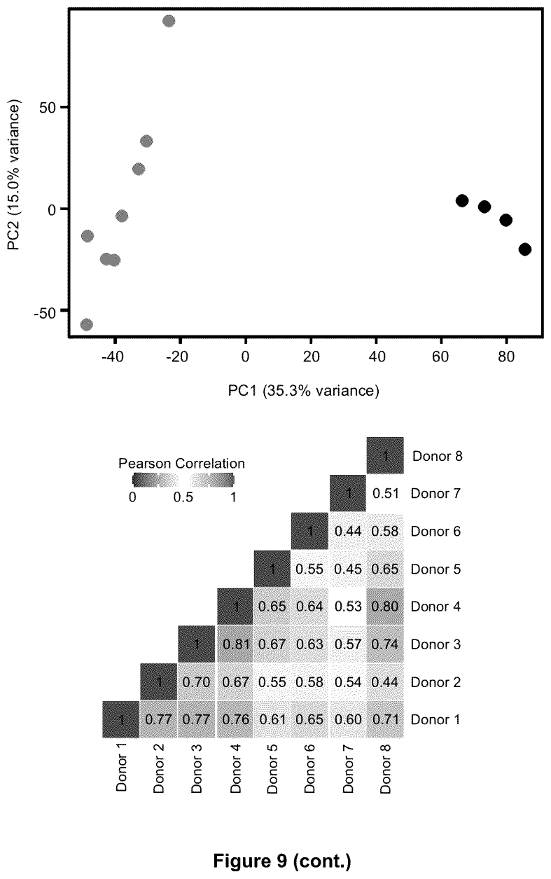

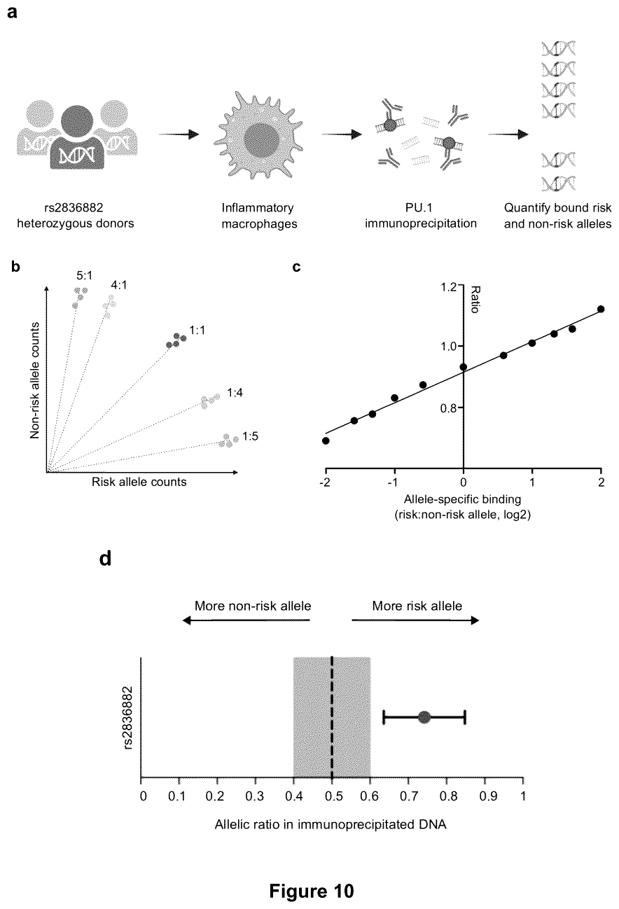

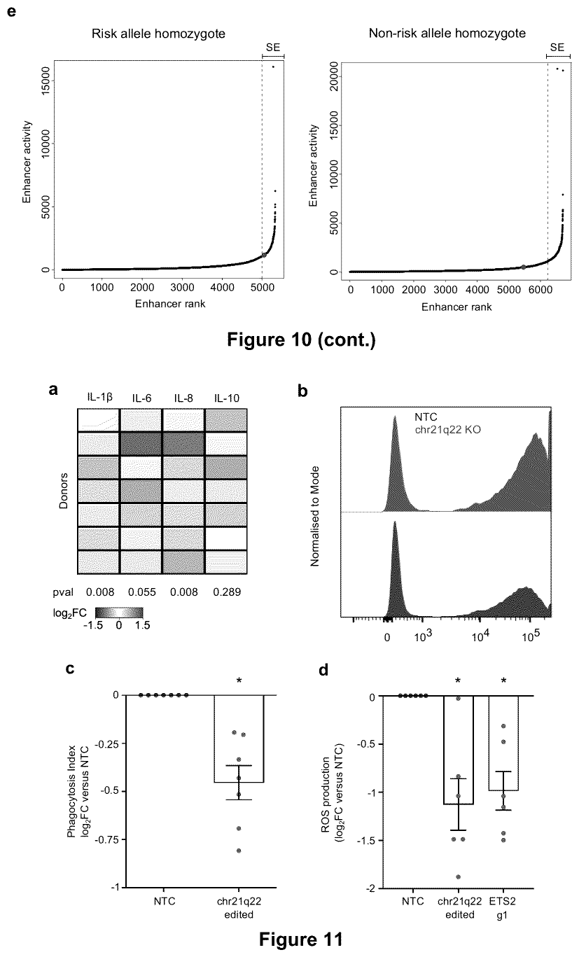

We next sought to identify the causal variant at chr21q22, whose biological effect was responsible for inflammatory disease risk. Unfortunately, statistical fine-mapping of the largest IBD GWAS to date [7] could not reduce the number of candidate variants due the very high correlation (linkage disequilibrium; LD) between them (Methods, FIG. 1e). We therefore used a functional approach (massively-parallel reporter assay; MPRA) to first identify the active enhancer sequences within the locus, and then determine if any candidate variants within this regulatory DNA might alter enhancer activity. MPRA is a high-throughput method that can simultaneously characterize enhancer activity in thousands of short DNA sequences by coupling each one to a uniquely barcoded reporter gene in an expression vector [17]. By normalising the barcode counts within mRNA, extracted from transfected cells, to the equivalent counts in the input DNA library, DNA sequences that modulate gene expression can be identified. We previously adapted MPRA for use in primary CD4+ T cells [18], and applied the same principles here to adapt it for primary macrophages-thereby ensuring that an appropriate repertoire of transcription factors was present to interact with the chr21q22 genomic sequences (FIG. 9). Based on the sequence of the 2-kb region containing all candidate genetic variants, we synthesised overlapping oligonucleotides (oligos; 114-bp genomic sequence tiled at 50-bp intervals) and included additional oligos with either risk or non-risk alleles for each variant. After cloning, the resulting vector library was transfected into inflammatory macrophages from multiple independent donors and high-throughput RNA and DNA sequencing was used to quantify barcode abundance (Methods). Using a sliding window analysis to summarise the expression-modulating effects of the tiling sequences, we identified a single 442-bp focus of enhancer activity (chr21: 40466236-40466677, hg19; FIG. 1f) which harboured three (of seven) candidate variants. Two of these variants were transcriptionally inert, but the third (rs2836882) had the strongest expression-modulating effect of any candidate variant at the locus, with the risk allele (G) significantly increasing transcription—consistent with the reported direction of the eQTL (FIG. 1f). Examining rs2836882 further, we noticed that this variant lies within an experimentally-confirmed PU.1 ChIP-seq peak in inflammatory macrophages (FIG. 1g). PU.1 is an important myeloid pioneer factor that can bind to heterochromatin, initiate nucleosome remodelling—thus enabling other transcription factors to bind—and activate transcription [20,21]. To determine whether rs2836882 genotype might affect PU. 1 binding, we identified two publicly-available macrophage PU 1 ChIP-seq datasets from heterozygous individuals, and used BaaIChIP to assess for allelic imbalances in PU. 1 binding. Despite not lying within a canonical PU.1 binding motif, marked allele-specific PU.1 binding was observed at rs2836882, with over 4-fold greater binding to the risk allele in both datasets (FIG. 1h). This result was replicated by immunoprecipitating PU. 1 in TPP macrophages from five rs2836882 heterozygotes and genotyping the bound DNA (FIG. 10). This suggests that the rs2836882 risk allele should confer greater enhancer activity—consistent with the MPRA and eQTL results. To test whether allele-specific differences in enhancer activity were present at the endogenous locus, we performed H3K27ac ChIP-seq in inflammatory macrophages from two major allele homozygotes and two minor allele homozygotes at rs2836882. While several nearby enhancer peaks were similar between these donors, the enhancer activity overlying rs2836882 was considerably stronger in major (risk) allele homozygotes (FIG. 1i), contributing to a ~2.5-fold increase in enhancer activity across the extended chr21q22 locus (FIG. 10). Altogether, these data reveal a genetic mechanism whereby the putative causal variant at chr21q22—identified via its functional consequences in primary macrophages—enhances binding of a pioneer transcription factor and increases the activity of a long-range ETS2 enhancer.

### Example 2-ETS2 is Required for Macrophage Inflammatory Responses

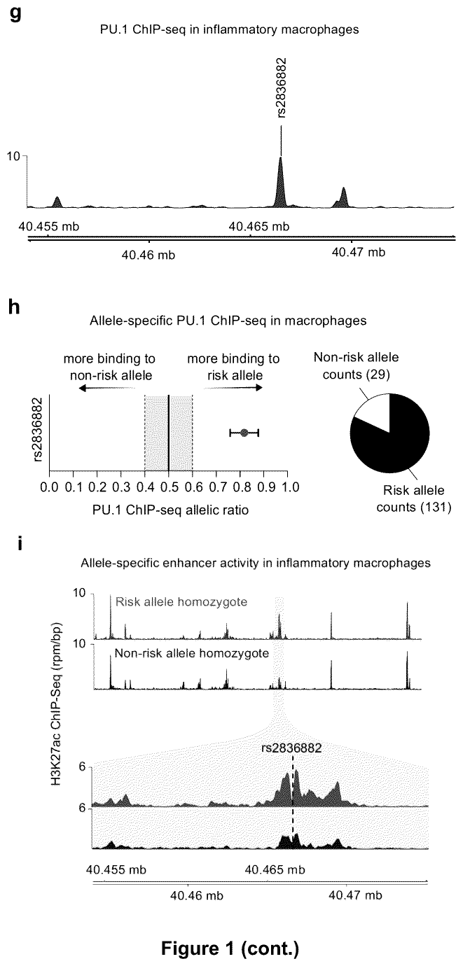

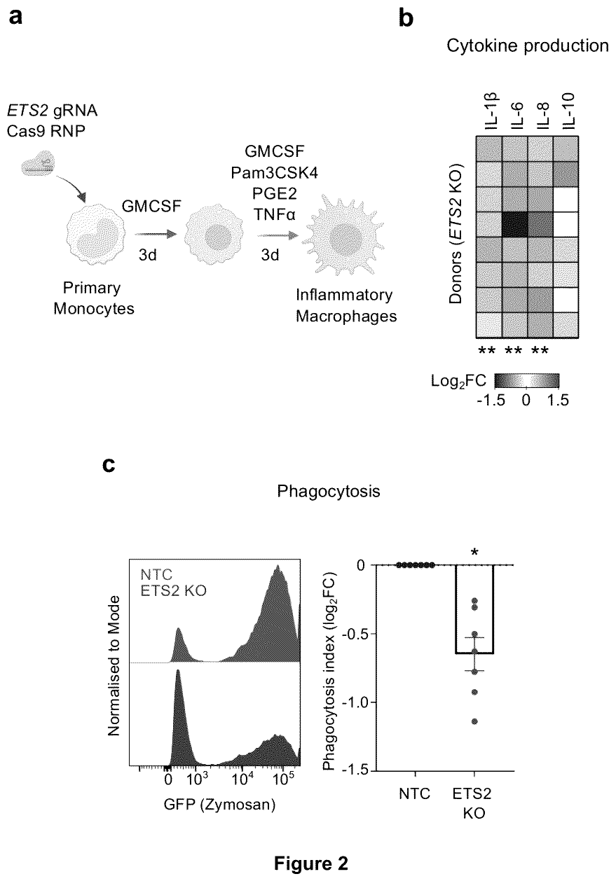

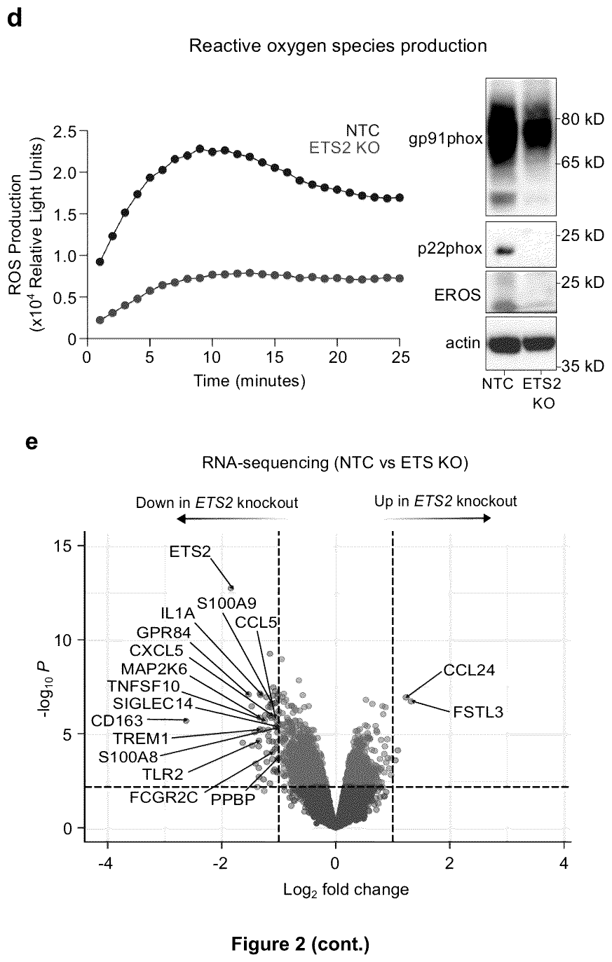

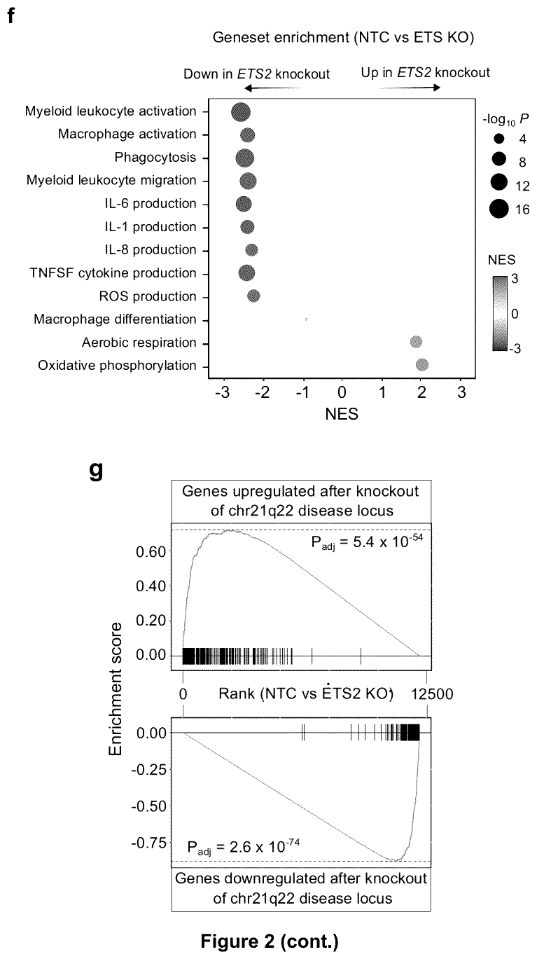

Having identified a plausible mechanism by which the chr21q22 risk haplotype increases ETS2 expression in monocytes/macrophages, we next sought to better understand the role of ETS2 in these cells. ETS2 is a member of the ETS family of transcription factors, which has been mainly studied as a proto-oncogene in cancer [23]. In contrast, the role of ETS2 in primary human macrophages has been less clearly defined, with previous studies using either cell-lines or complex mouse models and largely focusing on single downstream molecules [24,25,26,27,28]. This has led to contradictory reports, with ETS2 being described as both necessary and redundant for macrophage development [29,30], and both pro- and anti-inflammatory [24,25,26,27,28]. To elucidate the specific role of ETS2 in inflammatory human macrophages—and determine how dysregulated ETS2 expression might contribute to disease—we first used a CRISPR-Cas9-based loss-of-function approach (FIG. 2a). To minimise the chance that any effect might be due to off-target editing, two gRNAs targeting different ETS2 exons were designed, validated and individually incorporated into Cas9 RNPs for transfection into primary monocytes, resulting in mean on-target editing in 90% and 79% of cells respectively (FIG. 7). No differences in cell viability or the expression of macrophage markers were observed with either gRNA, suggesting that ETS2 was not required for inflammatory macrophage differentiation (FIG. 7). In contrast, production of pro-inflammatory cytokines, including IL-6, IL-8 and IL-1β, was significantly reduced following ETS2 deletion (FIG. 2b), although IL-10—an anti-inflammatory cytokine—was less affected. TNFα could not be assessed as it had been added exogenously. We next investigated whether ETS2 might also be required for other macrophage effector functions. First, we examined phagocytosis using a fluorescently-labelled substrate (zymosan particles) that can be detected by flow cytometry. Similar to pro-inflammatory cytokine production, phagocytosis was significantly impaired following ETS2 editing (FIG. 2c). Next, we measured extracellular reactive oxygen species (ROS) production—a key effector response that contributes to tissue damage in inflammatory disease [31]. We found that disrupting ETS2 profoundly reduced the oxidative burst following macrophage activation—an effect that appeared to be due to reduced expression of key components of the NADPH oxidase (FIG. 2d, FIG. 11). Altogether, this suggests that ETS2 is required for multiple effector functions in inflammatory macrophages.

To better understand the molecular basis for these distinct functional effects, we performed whole transcriptome RNA-sequencing (RNA-seq) in ETS2 edited and control inflammatory macrophages from multiple independent donors. Disruption of ETS2 led to widespread transcriptional changes, with significantly reduced expression of many inflammatory genes, including several key initiators and amplifiers of inflammation (FIG. 2e). Affected gene classes included cytokines (e.g. TNFSF10/TRAIL, TNFSF13, IL1B), chemokines (e.g. CXCL1, CXCL3, CXCL5, CCL2, CCL5), secreted effector molecules (e.g. S100A8, S100A9, MMP14, MMP9), cell surface receptors (e.g. FCGR2A, FCGR2C, TREM1), pattern recognition receptors (e.g. TLR2, TLR6, NOD2), and signalling molecules (e.g. MAP2K, GPR84, NLRP3). To better characterise the pathways affected by ETS2 deletion, we performed gene-set enrichment analysis (GSEA) using the Gene Ontology Biological Pathways dataset. This corroborated the functional effects we had observed (FIG. 2f), with the most negatively-enriched pathways (downregulated following ETS2 disruption) being those involved in macrophage activation, proinflammatory cytokine production, phagocytosis and ROS production. Genes involved in macrophage migration were also significantly downregulated, but gene sets relating to monocyte-to-macrophage differentiation were not significantly affected-consistent with ETS2 directing macrophage inflammatory responses, but not influencing monocyte-to-macrophage development. Although fewer genes were upregulated following ETS2 deletion (FIG. 2e), two notable positively-enriched pathways related to aerobic respiration and oxidative phosphorylation (OXPHOS; FIG. 2f)—metabolic processes that have been previously linked to anti-inflammatory macrophage behaviour [32]. Collectively, these data identify an indispensable role for ETS2 in a range of macrophage effector functions, which could explain why dysregulated ETS2 expression contributes to multiple inflammatory diseases. Indeed, deletion of the disease-associated chr21q22 enhancer phenocopied both the functional and transcriptional consequences of ETS2 disruption (FIG. 2g, FIG. 11).

### Example 3—ETS2 Controls Macrophage Inflammatory Responses

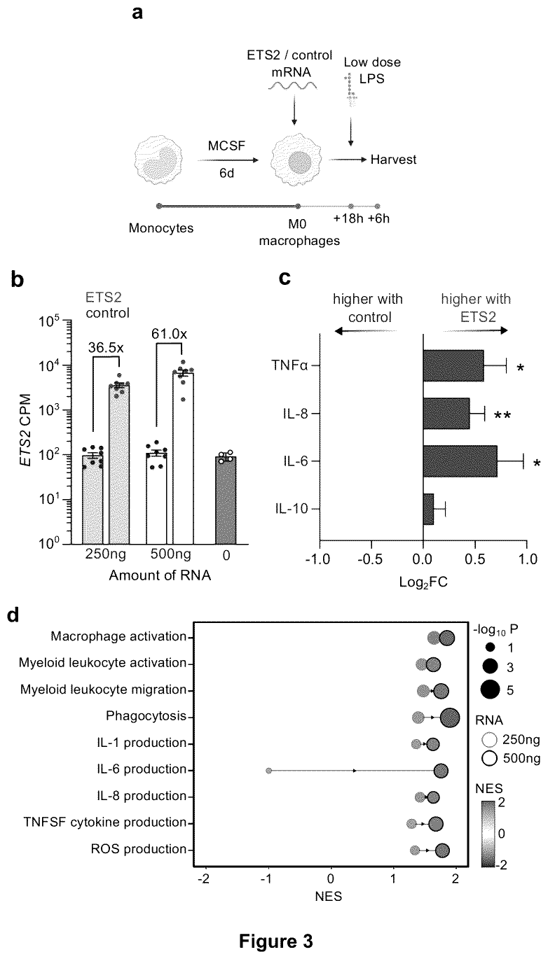

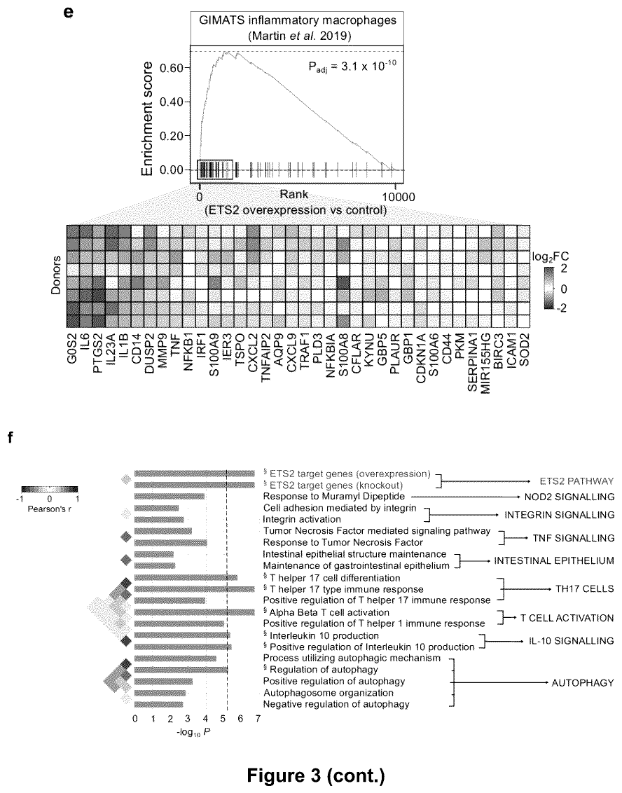

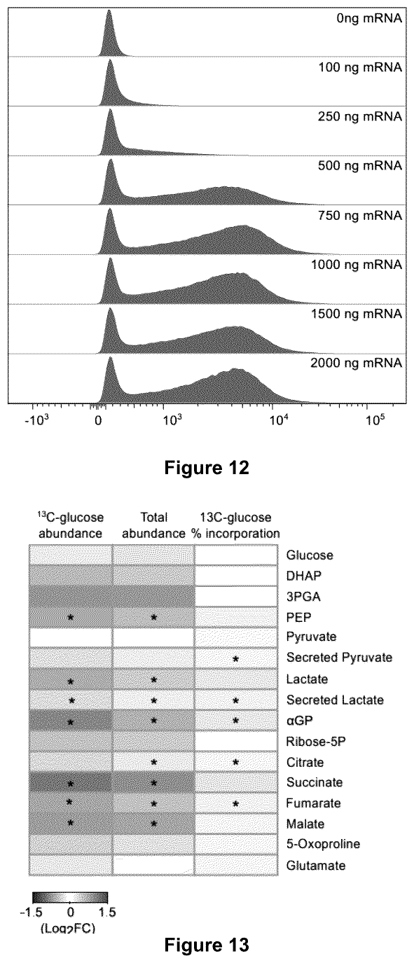

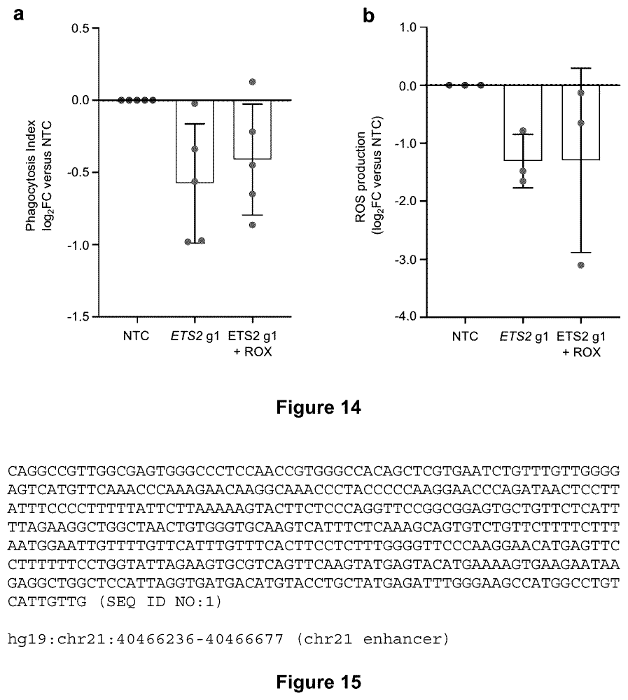

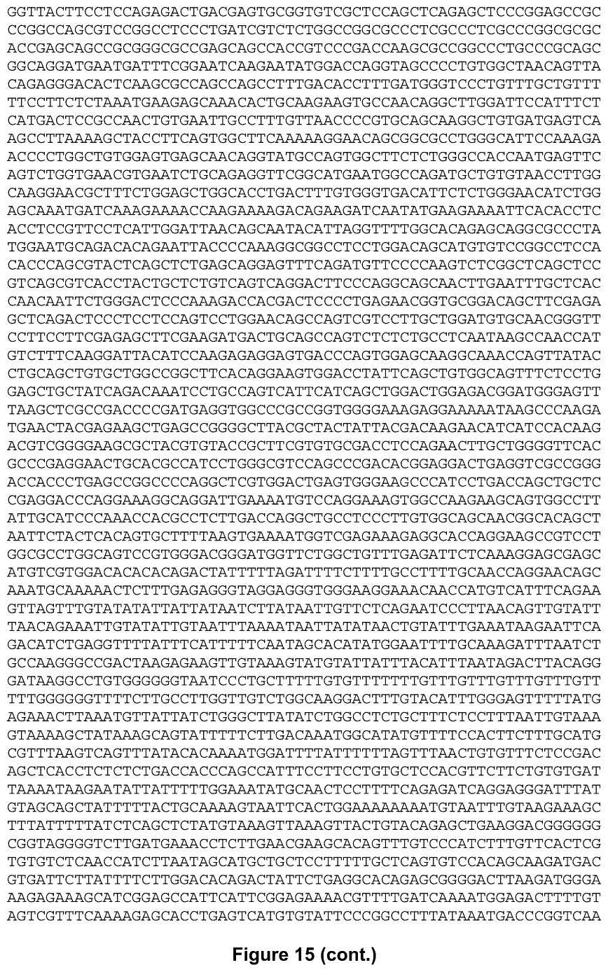

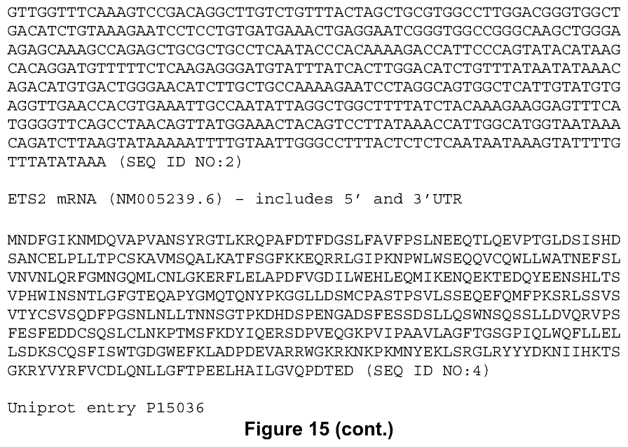

The discovery that ETS2 was necessary for effector functions in monocyte-derived macrophages raised the question of whether it might also be sufficient to drive them—as would be expected of a central controller of inflammatory responses. This is particularly important because although loss-of-function approaches are useful for identifying a gene's biological roles, the chr21q22 risk haplotype increases ETS2 expression. To address this, we first optimised a method to induce controlled overexpression of specific genes in resting, non-activated (M0) macrophages by transfecting pre-defined amounts of in vitro transcribed mRNA (FIG. 3a, FIG. 12). To minimise non-specific activation relating to the transfected RNA, in vitro transcription was performed using co-transcriptional capping (to minimise uncapped products), and incorporating modified, minimally immunogenic nucleotides (replacing uridine with N1-methyl-pseudouridine and cytidine with methylcytidine). Control cells were transfected with equivalent amounts of mRNA encoding the reverse complement of ETS2—thereby controlling for the quantity, length and purine/pyrimidine composition of the transfected RNA but with a transcript that would not be translated (FIG. 3b). ETS2 expression in these control cells was comparable to untransfected cells cultured under similar conditions, indicating that this approach did not reduce ETS2 mRNA levels. After RNA transfection, cells were exposed to low-dose lipopolysaccharide for 6 hours to initiate a low-grade inflammatory response (FIG. 3a). We first measured cytokine secretion in the cell culture supernatants and found that ETS2 overexpression was sufficient to increase production of several proinflammatory cytokines, although again IL-10 was less affected (FIG. 3c). To better characterise the consequences of ETS2 overexpression, we performed RNA-seq and specifically examined the macrophage activation pathways that we had previously shown required ETS2. Strikingly, not only were multiple inflammatory pathways induced by ETS2 overexpression-including macrophage activation, proinflammatory cytokine production, ROS production, phagocytosis and migration—but this induction was shown to be dose-dependent, with greater enrichment of every pathway when more ETS2 mRNA was transfected (FIG. 3d). This demonstrates that ETS2 is both necessary and sufficient for inflammatory responses in primary human macrophages, revealing it to be a central controller of myeloid effector functions whose dysregulation is directly linked to human disease.

### Example 4—ETS2-Regulated Genes Play a Central Role in IBD

To understand whether ETS2 might directly contribute to the macrophage phenotype observed in inflammatory disease, we compared the transcriptional consequences of overexpressing ETS2 with a gene signature derived from intestinal macrophages in Crohn's disease-one of the conditions linked to chr21q22. Single cell RNA-seq analysis has previously shown that active Crohn's disease is characterised by an expanded population of inflammatory monocyte-derived macrophages that contributes to anti-TNFα resistance [33]. Using the Crohn's disease macrophage signature as a gene set, we found overexpressing ETS2 in resting macrophages induced a transcriptional state that closely resembled inflammatory disease macrophages, with core (“leading edge”) enrichment of over half of the genes in the signature, including many targets of approved therapies (FIG. 3e).

Based on the importance of ETS2 in macrophage inflammatory responses, and the fact that ETS2 overexpression phenocopied the disease-associated inflammatory state, we hypothesised that other genetic associations might also affect this previously uncharacterised pathway. An important goal of GWAS was to identify central disease pathways [3], but this has proven challenging due to a paucity of confidently-identified causal genes and a limited understanding of how these are affected by genetic variation [3]. There have, however, been some notable successes, including the discovery that autophagy is involved in Crohn's disease susceptibility via genetic associations in several autophagy genes [34]. To better characterise the genetic risk attributable to the macrophage ETS2 pathway, we focused on IBD since this has considerably more genetic associations than any other chr21q22-associated disease. Examining the list of commonly downregulated genes following ETS2 editing (Padj <0.05 for both gRNAs), we identified over 20 IBD risk genes—including many that have been proposed to be causal at their respective loci [7,35] (Table Sx). These included several genes that are thought to affect macrophage biology (e.g. SP140, LACC1/FAMIN, CCL2, CARD9, CXCL5, TLR4, SLAMF8) as well as some that are highly expressed in macrophages but not previously linked to specific pathways (e.g. ADCY7, PTPRC, TAGAP, PTAFR, PDLIM5, DOK2). To more formally assess the extent to which ETS2-driven inflammation is implicated by IBD genetics—relative to previously-identified disease pathways—we used SNPsea [36], an algorithm designed to identify pathways affected by disease loci. 241 IBD-associated genetic loci were tested for enrichment in 7,660 pathways, comprising 7,658 Gene Ontology Biological Pathways and 2 overlapping lists of ETS2-regulated genes (either those downregulated following ETS2 editing or upregulated following ETS2 overexpression). Significance of enrichment was empirically computed using 5 million matched null SNP sets, and pathways previously implicated by IBD genetics were extracted for comparison. Strikingly, ETS2 target genes-however they were defined-were more strongly enriched for IBD-associated loci than almost all previously implicated pathways, with no null SNP set showing greater enrichment than either of the ETS2-regulated genelists. After applying a stringent Bonferroni multiple-testing correction, only ETS2-regulated genes and IBD pathways relating to T cell activation, T-helper 17 cells, autophagy and IL-10 signalling showed significant enrichment (FIG. 3f). This suggests that ETS2 signalling in inflammatory macrophages plays a central role in IBD pathogenesis, with greater genetic enrichment than many previously implicated pathways.

### Example 5—ETS2 Orchestrates Macrophage Inflammatory Responses Via Transcriptional and Metabolic Effects

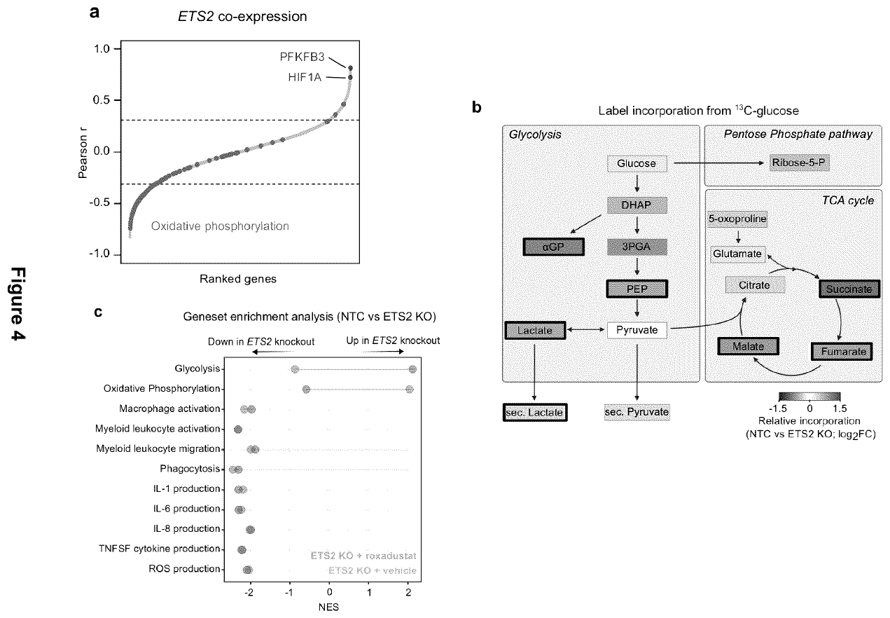

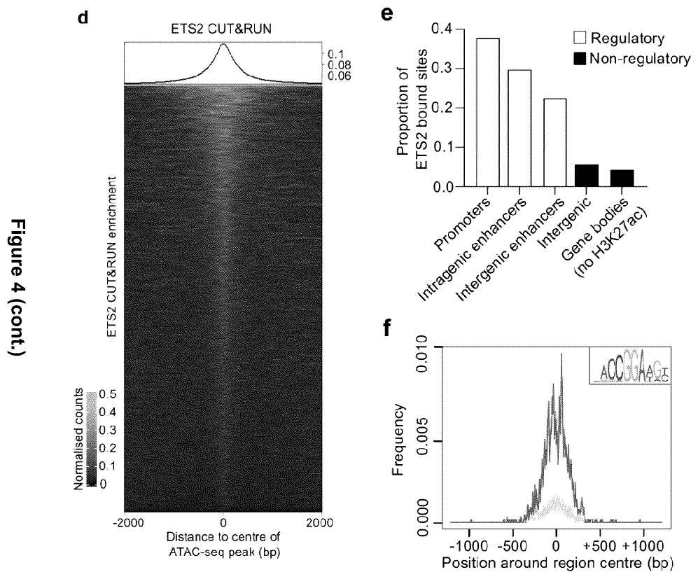

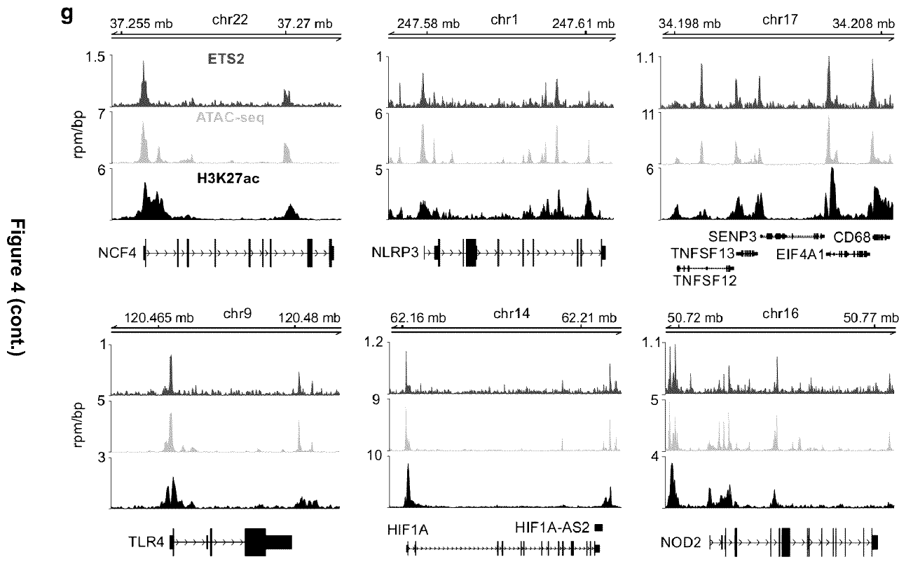

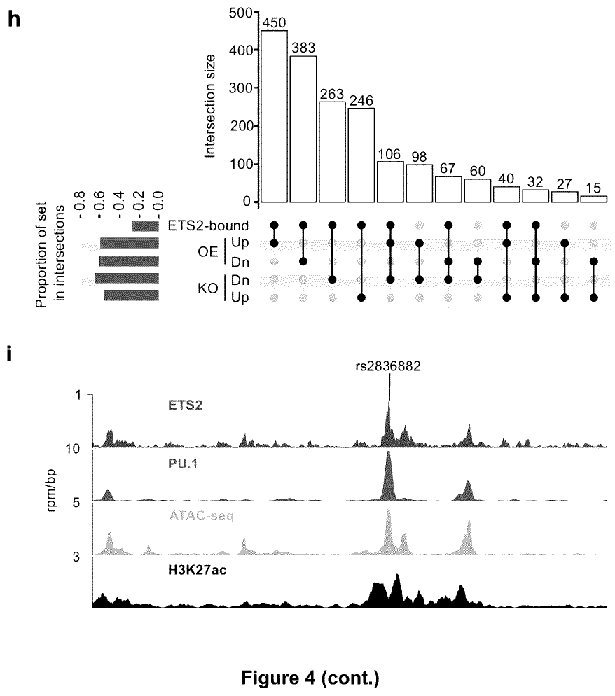

We next sought to understand how ETS2 might control such diverse effector functions in macrophages. Studying ETS2 biology is challenging because no ChIP-seq-grade antibodies are available, precluding direct identification of its transcriptional targets. Even the ENCODE project, which performed ChIP-seq for 181 transcription factors, was unable to directly immunoprecipitate ETS2 [37]. We therefore first used a “guilt-by-association” approach to identify genes that were highly co-expressed with ETS2 across 64 different macrophage polarisation conditions15. This identified PFKFB3—encoding the rate-limiting enzyme of glycolysis—as the most strongly co-expressed gene with HIF1A also being highly co-expressed (FIG. 4a). Together, these genes are known to facilitate a “glycolytic switch” that is essential for myeloid inflammatory responses [38]. We therefore hypothesised that ETS2 might control inflammatory responses via metabolic reprogramming—an idea supported by OXPHOS genes being negatively correlated with ETS2 (FIG. 4a) and upregulated following ETS2 deletion (FIG. 2f). To characterise the metabolic consequences of disrupting ETS2, we quantified label incorporation from 13C-glucose in CRISPR-edited and unedited inflammatory macrophages using gas chromatography-mass spectrometry (GC-MS). Widespread modest reductions in both unlabelled and labelled glucose metabolites were detected following ETS2 disruption (FIG. 4b, FIG. 13). This affected both glycolytic and TCA cycle metabolites, including significant reductions in intracellular and secreted lactate, a hallmark of anaerobic glycolysis, and succinate, an important inflammatory signalling metabolite [39]. These results are consistent with the reported effects of suppressing glycolysis, with the reductions in TCA metabolites being due to consumption by mitochondrial OXPHOS [40,41]. To determine whether these metabolic effects might be responsible for the ETS2-mediated inflammatory effects, we treated ETS2-edited inflammatory macrophages with roxadustat, a HIF1 stabiliser that can promote glycolysis via HIF1α-mediated metabolic reprogramming. This had the predicted effect on expression of genes involved in glycolysis and OXPHOS, but did not rescue the effects of ETS2 disruption, either transcriptionally or functionally (FIG. 4c, FIG. 13). Consequently, while disrupting ETS2 does appear to alter glucometabolism, these effects are not solely responsible for the observed differences in inflammation.

We therefore revisited whether we could identify ETS2 target genes—to try to understand how ETS2 could control such diverse inflammatory effects. Using a range of anti-ETS2 antibodies, we confirmed that none worked for ChIP (data not shown) and so investigated whether any might work for Cleavage-Under-Targets-and-Release-Using-Nuclease (CUT&RUN), which does not require formaldehyde fixation. One of these antibodies identified multiple, significantly-enriched genomic regions (peaks) of which 6,560 were reproducibly detected across two biological replicates (Irreproducible Discovery Rate <0.01) with acceptable quality metrics (average FRIP score 0.23) (FIG. 4d). These peaks were predominantly located in active regulatory regions (90% in promoters or active enhancers, FIG. 4e) and were highly enriched for a canonical ETS2 position weight matrix (3.98-fold enrichment over global controls, P=4.16e−126, FIG. 4f)—consistent with being sites of ETS2 binding. After combining the biological replicates to improve peak detection, we detected ETS2 binding peaks at the promoters of several key inflammatory genes, indicating that ETS2 is likely to directly regulate multiple macrophage effector responses (FIG. 4g). In support of this, 48.3% of genes dysregulated following ETS2 editing, and 50.3% of genes dysregulated following ETS2 overexpression, contained an ETS2 binding peak within their core promoter or its putative cis-regulatory elements (FIG. 4h). These transcriptional targets included HIF1A, PFKFB3, and other glycolytic genes (e.g. GPI, HK2, and HK3), suggesting that the observed metabolic changes might also be directly induced by ETS2, rather than solely due to differences in inflammation. Remarkably, we also detected ETS2 binding at its own enhancer at chr21q22 (FIG. 4i). This is consistent with previous reports that PU.1 and ETS2 can interact synergistically [43], and would implicate a feed-forward mechanism at the disease-associated locus, where increased ETS2 expression reinforces ETS2 enhancer activity. Collectively, these data implicate ETS2 as a master regulator of monocyte/macrophage responses during chronic inflammation, capable of directing a multi-faceted transcriptional programme and contributing to a metabolic environment that is permissive for inflammation.

### Example 6—ETS2-Driven Inflammation is Detectable in Diseased Tissue and can be Targeted Pharmacologically

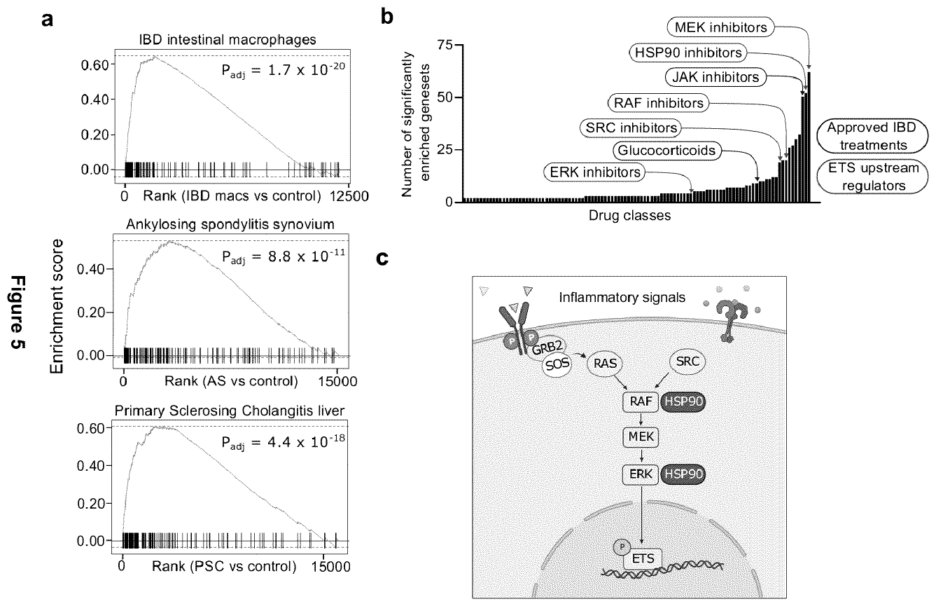

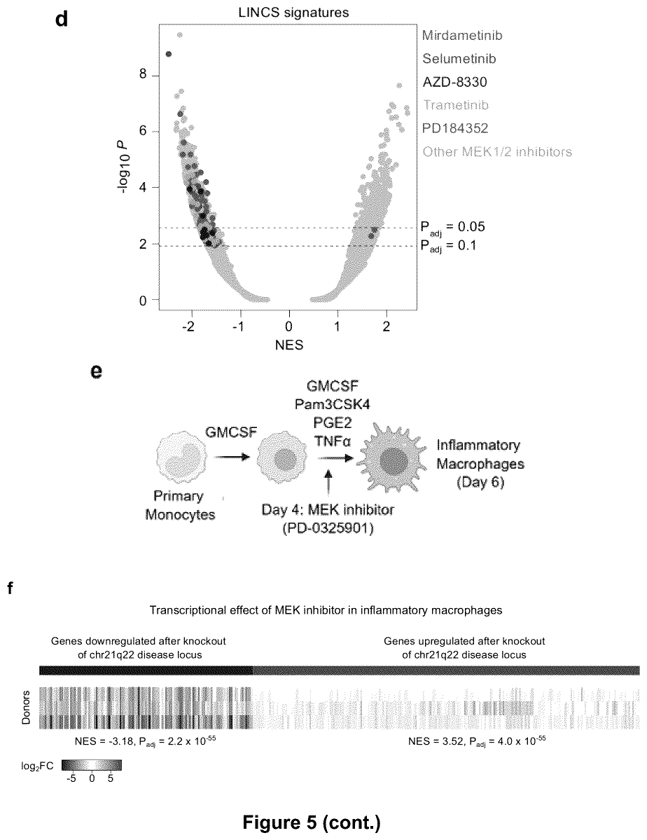

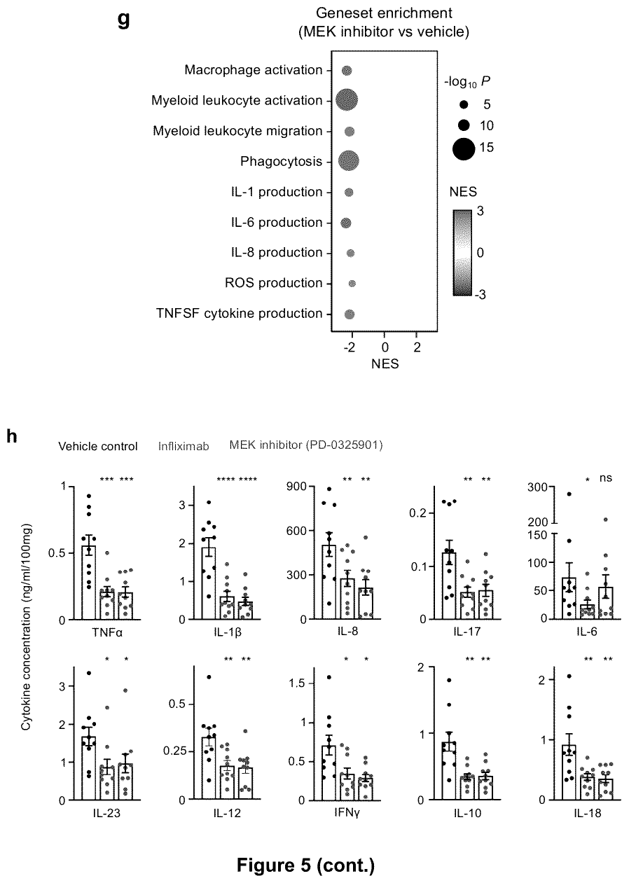

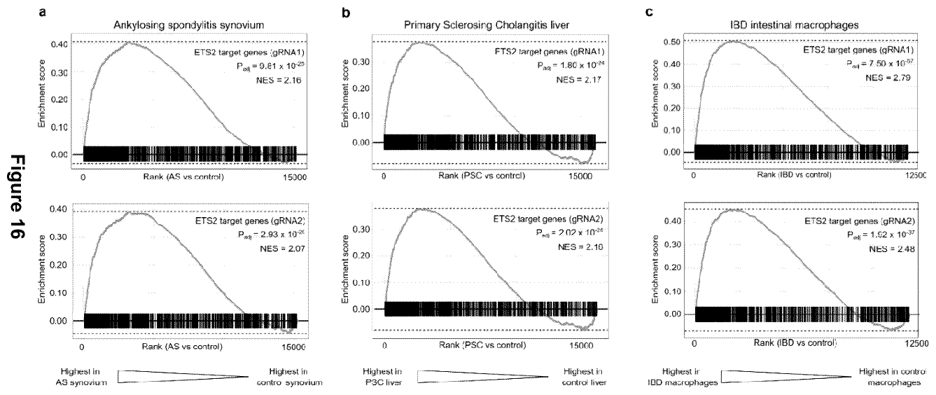

The strong enrichment of IBD GWAS hits in ETS2-regulated genes led us to hypothesise that the transcriptional footprint of this pathway might be generally detectable in the affected organs of chr21q22-associated diseases—a possibility that would have important therapeutic implications. Using publicly-available gene expression data from diseases linked to chr21q22-intestinal macrophages from IBD, synovium from ankylosing spondylitis, and liver from PSC-we confirmed that diseased tissue was significantly enriched for genes regulated by ETS2 (FIG. 5a, FIG. 16). We therefore investigated whether this pathway could be pharmacologically targeted. Specific ETS2 inhibitors do not exist and structural analyses indicate that there is no allosteric inhibitory mechanism that could be easily targeted [44]. We therefore used the NIH LINCS database to identify drugs that might modulate ETS2 activity [45]. This repository contains over 23,000 differentially-expressed gene lists from cell-lines exposed to over 6,000 small molecules. Using GSEA, 906 drug signatures were found to mimic the transcriptional effect of disrupting ETS2 in inflammatory macrophages (Padj <0.05), including several drugs already approved for treating IBD and AS (e.g. JAK inhibitors). Of these candidate therapies, the most common class were MEK inhibitors (FIG. 5b), which are currently licensed for non-inflammatory human diseases (e.g. neurofibromatosis). This result was not due to a single compound, but was rather a class effect with multiple MEK1/2 inhibitors being capable of downregulating ETS2 target genes (FIG. 5c). This made biological sense, since MEK1 and MEK2-together with several other targets identified—are known upstream regulators of ETS-family transcription factors (FIG. 5d). Indeed, some of these drug classes, including MEK and HSP90 inhibitors, have been reported to be beneficial in mouse colitis models, although this is often a poor indicator of clinical efficacy—with several approved IBD treatments being ineffective in mice and many drugs that improve mouse models being ineffective in human IBD [46]. To determine whether MEK inhibition would abrogate inflammatory responses in primary human macrophages, we differentiated monocytes under chronic inflammatory conditions and treated them with a selective, non-ATP competitive MEK inhibitor (PD-0325901; FIG. 5e). We observed potent anti-inflammatory activity that phenocopied the effect of disrupting ETS2 or deleting the chr21q22 enhancer (FIG. 5f, FIG. 17), with downregulation of multiple inflammatory pathways (including several approved drug targets; FIG. 5g). Given the limitations of animal colitis models [46], we further investigated the therapeutic potential of targeting ETS2 signalling using a human gut explant model. To do this, intestinal mucosal biopsies were taken from patients with active IBD, who were not receiving immunosuppressive or biologic therapies, and cultured on a transwell insert [47] for 18 hours with either a MEK inhibitor or a negative or positive control (Methods). Release of multiple IBD-associated inflammatory cytokines was significantly reduced by MEK inhibition—to comparable levels observed with infliximab (an anti-TNFα monoclonal antibody widely used for IBD; FIG. 5h). Moreover, we confirmed that expression of ETS2-regulated genes was reduced (FIG. 5i) and that there was significant improvement in a validated transcriptional inflammation score [48] that reflects IBD-associated inflammation and has been shown to reduce upon effective therapy (FIG. 5j). Together, this shows that targeting an upstream regulator of ETS2 can abrogate pathological inflammation in a chr21q22-associated disease, and may be useful therapeutically.

### Example 7—Antibody-Drug Conjugate (ADC) Experiments

We selected CD163 as an optimal target for ADC-mediated drug delivery due to its restricted-yet ETS2-dependent-expression in inflammatory macrophages, scavenger function, and prior literature in ADCs.

We will test cleavable and non-cleavable linkers, via the maleimide conjugation method used in most approved ADCs, to generate different drug: antibody ratios (DAR 4, 6 and 8). This will optimise the trade-off between hydrophobicity/aggregation and therapeutic effect.

The cleavable linker selected is glucuronide-based (GlyPro). This is stable in circulation and selectively cleaved by β-glucuronidase-thereby facilitating selective release in lysosomes. Additionally, this linker has advantageous hydrophilic properties, which should reduce aggregation and promote solubility. Such linkers have been successfully employed in several pro-drugs. The non-cleavable linker selected is maleimide-PEG3-based. These linkers perform significantly better in in vivo studies, with a lower risk of premature drug release in non-target cells and lower toxicity. This should improve the stability, tolerability, and therapeutic window, with the drug release only occurring following antibody/linker degradation in lysosomes of CD163-expressing cells.

### Example 8—Esterase Sensitive Motif (ESM)-Conjugated MEK Inhibitor Experiments

Unlike most human carboxylesterases, which are ubiquitously expressed, human carboxylesterase-1 (hCE-1) is predominantly expressed in monocyte-derived cells (e.g. monocytes and macrophages). ESM technology uses esters that are selectively hydrolysed by hCE-1, resulting in intracellular accumulation of the pharmacologically active acid (drug) in hCE-1-expressing cells because, unlike the ester, the acid cannot readily diffuse out. This approach can facilitate a 1000-fold increase in potency over a non-conjugated inhibitor. hCE-1 is also expressed in the liver and thus an intermediate rate of hydrolysis-which can be modulated by varying the ESM and inhibitor—is required to ensure enough ester avoids first-pass metabolism. We will synthesise ESM-MEK inhibitor conjugates with various ESMs, linker lengths and MEK inhibitors to identify the optimal combination for downstream biological testing. Corresponding acid products and non-hydrolysable ester controls will be synthesised as controls.

Specifically, we will test:
3 MEK inhibitors (to identify the most suitable candidate based on pharmacological activity of the acid-drug and synthetic tractability).
2 linker lengths (these link to the amino acid group and are typically either one or two carbons. This is an important to ensure the molecule extends through the esterase active site).
2 ESMs (the two validated ESMs described in the literature: cyclopentyl L-leucinate and cyclopentyl(S)-2-amino-2-cyclohexylacetate).

### Example 9—Biochemical and Functional Validation of MEKi-Conjugated Molecules

On-target binding: a competitive binding assay will test whether the ADC binds CD163 (using a fluorescently-tagged ADC and measuring binding in the presence of the varying concentrations of the unconjugated antibody). Other macrophage markers such as CD209 (DC-SIGN) and CD206 (MRC1) will also be tested.

Internalisation: Internalisation of a fluorescently-tagged ADC in inflammatory macrophages will be tested by confocal microscopy.

ESM-MEKᵢ—Selective hydrolysis: conjugates will be incubated with peripheral blood mononuclear cells before individual cell populations are purified (e.g. T cells, B cells, NK cells and monocytes) and mass spectroscopy performed (HPLC-MS/MS) to determine the intracellular abundance of the parent ester and hydrolysed acid. Hydrolysis rates will be quantified by incubating ESM-MEKi conjugates with primary monocytes (hCE-1 positive) and hCE-1-deficient control cells.

Pharmacological activity (biochemical): The MEK inhibition IC50 for each ADC and cleaved drug or each ESM-MEKi and acid-drug will be calculated using a radioactive filter binding assay with 33P ATP.

Pharmacological activity (physiological): ERK1/2 phosphorylation (Thr202/Tyr204) will be quantified by flow cytometry in individual leukocyte populations following PBMC culture with varying concentrations of the conjugate or cleaved drug.

### Example 10—Materials and Methods

### Analysis of Existing Data Relating to Chr21q22

We used IBD GWAS summary statistics [7] to perform multiple causal variant fine-mapping using susieR[49] with reference minor allele and LD information calculated from 503 European samples from 1000 Genomes phase 3. All R analyses used v.4.2.1. Palindromic SNPs (A/T or C/G) and any SNPs that didn't match by position or alleles were pruned before imputation using the ssimp equations reimplemented in R. This did not affect any candidate SNP at chr21q22. We obtained SuSIE fine mapping results for ETS2 (with identifier ENSG00000157557 or ILMN_1720158) in monocyte datasets from the eQTL Catalog. Colocalisation analysis was performed using coloc v5.2.0 using a posterior probability of H4 (PP.H4.abf)>0.5 to call colocalisation.

Raw H3K27ac ChIP-seq data from primary human immune cells were downloaded from Gene Expression Omnibus (GEO series GSE18927 and GSE96014) and processed as described previously [51]. Processed promoter-capture Hi-C data [13] from 17 primary immune cell-types were downloaded from OSF (https://osf.io/u8tzp).

### Monocyte Purification and Macrophage Differentiation

Leukocyte cones from healthy donors were obtained from NHS Blood and Transplant (Cambridge Blood Donor Centre, Colindale Blood Centre, or Tooting Blood Donor Centre). Peripheral blood mononuclear cells (PBMCs) were isolated by density centrifugation (Histopaque 1077, Sigma) and monocytes were positively selected using CD14 Microbeads (Miltenyi Biotec). Macrophage differentiation was performed using conditions that model chronic inflammation (TPP) [15]: 3 days GM-CSF (50 ng/ml, Peprotech) followed by 3 days GM-CSF, TNFα (50 ng/ml, Peprotech), PGE2 (1 μg/ml, Sigma Aldrich), and Pam&CSK4 (1 μg/ml, Invivogen). All cultures were performed at 37° C., 5% CO₂ in antibiotic-free RPMI1640 media containing 10% FBS, GlutaMAX, and MEM Non-Essential Amino Acids (all ThermoFisher). Cells were detached using Accutase (Biolegend).

### Identifying a Model of Chronic Inflammatory Macrophages

Human monocyte-derived macrophage gene expression data files (n=299) relating to 64 different polarisation conditions were downloaded from Gene Expression Omnibus (GSE47189) and quantile normalised. Data from biological replicates were summarised to the median value for every gene. Gene set variation analysis (using the GSVA package in R) was performed to identify the polarisation condition that most closely resembled CD14+ monocytes/macrophages from active IBD-using disease-associated lists of differentially-expressed [53].

### CRISPR-Cas9 Editing of Primary Human Monocytes

gRNA sequences were designed using CRISPick (formerly GPP sgRNA Designer) and synthesised by IDT. gRNA sequences: chr21q22 5′ gRNA, CCUGGCUGCCUCGCGUUUCC; chr21q22 3′ gRNA, CCUCGUCCAACAGAGAGCAA; ETS2 gRNA1, CAGACACAGAAUUACCCCAA; ETS2 gRNA2, UUGCUGCACGGGGUUAACAA. Alt-R CRISPR-Cas9 Negative Control crRNA #1 (IDT) was used as a non-targeting control. Cas9-gRNA ribonucleoproteins were assembled as described previously and nucleofected into 5×10⁶ monocytes in 100 μL nucleofection buffer (Human Monocyte Nucleofection Kit, Lonza) using a Nucleofector 2b (Lonza, program Y-001). After nucleofection, monocytes were immediately transferred into 5 ml of pre-warmed culture media in a 6-well flat-bottomed plate, and differentiated into macrophages under TPP conditions. Editing efficiency was quantified by PCR amplification of the target region in extracted DNA (chr21q22_Fw primer, GGTGGGGAGAGTTCCAAAGG; chr21q22_Rv, TCACCCTTCACCTCTTTGCT; ETS2_g1_Fw, TCCTGAAGGTCCCATGAAAG; ETS2_g1_Rv, TCATTATGGCTCTGGGGTTC; ETS2_g2_Fw, GCGGCACATTCATATCACAC; ETS2_g2_Rv, GCAGAATACCCCAAGCAAAA). Editing efficiency at the chr21q22 locus was measured via quantification of amplified fragments (2100 Bioanalyzer, Agilent) as previously described [51]. Editing efficiency for individual gRNAs was assessed using the Inference of CRISPR Edits tool (ICE, Synthego).

### PrimeFlow RNA Assay

RNA abundance was quantified by PrimeFlow (ThermoFisher) in chr21q22-edited and unedited (NTC) cells on days 0, 3, 4, 5, and 6 of TPP differentiation. Target probes specific for ETS2 (Alexa Fluor 647), BRWD1 (Alexa Fluor 568) and PSMG1 (Alexa Fluor 568) were used according to the manufacturer's instructions. Data were analysed using FlowJo v10 (BD Biosciences)

### MPRA

Overlapping oligonucleotides containing 114-nt of genomic sequence were designed to tile the region containing chr21q22 candidate SNPs (99% credible set) at 50 bp intervals. Six technical replicates were designed for every genomic sequence, each tagged by a unique 11-nt barcode. Additional oligonucleotides were included to test the expression-modulating effect of every candidate SNP in the 99% credible set. Allelic constructs were designed as described previously and tagged by 30 unique 11-nt barcodes. Positive and negative controls were included as described previously [51]. 170-nt oligonucleotides were synthesised as part of a larger MPRA pool (Twist Biosciences) containing the 16-nt universal primer site ACTGGCCGCTTCACTG, 114-nt variable genomic sequence, KpnI and XbaI restriction sites (TGGACCTCTAGA), an 11-nt barcode, and the 17-nt universal primer site AGATCGGAAGAGCGTCG. Cloning into the MPRA vector was performed as described previously [51]. A suitable promoter for the MPRA vector (RSV) was identified by testing promoter activities in TPP macrophages. The MPRA vector library was nucleofected into TPP macrophages (5 μg vector into 5×106 cells) in 100 μl nucleofection buffer (Human Macrophage Nucleofection Kit, Lonza) using a Nucleofector 2b (program Y-011). To ensure adequate barcode representation, a minimum of 2×10⁷ cells were nucleofected for every donor (n=8). After 24 hours, RNA was extracted and sequencing libraries were made from mRNA or DNA input vector as described previously [51]. Library pools (of 6 samples each) were sequenced on an Illumina HiSeq2500 high output flow-cell (50 bp, single-end reads) and data were pre-processed as previously described [51]. To identify regions of enhancer activity, a paired t-test was performed to identify genomic sequences that enhanced transcription. A sliding window analysis (300-bp window) was then performed across all tiling sequences using the les package in R. Expression-modulating variants were identified using QuASAR-MPRA [55], as described previously [51].

### BaaIChIP

Publicly-available PU. 1 ChIP-seq datasets from human macrophages were downloaded from GEO, and BAM files were examined (using the IGV genome browser) to identify rs2836882 heterozygotes (i.e. files containing both A and G allele reads at chr21: 40,466,570, hg19). Two suitable samples were identified (GSM1681423 and GSM 1681429) which were used for a Bayesian analysis of allelic imbalances in PU.1 binding, with correction for biases introduced by overdispersion and biases towards the reference allele—implemented in the BaaIChIP package [22] in R.

### Allele-Specific PU.1 ChIP-Genotyping

A 100 ml blood sample was taken from five healthy individuals who were heterozygous at rs2836882 (assessed via Taqman genotyping, ThermoFisher). All participants provided written informed consent. Ethical approval was provided by the London-Brent Regional Ethics Committee (REC: 21/LO/0682). Monocytes were isolated from PBMC using CD14 Microbeads (Miltenyi Biotec) and differentiated into inflammatory macrophages using TPP conditions [15]. Following differentiation, macrophages were detached using Accutase and cross-linked for 10 min in fresh media containing 1% formaldehyde. Cross-linking was quenched with glycine for 5 min (final concentration 0.125M). Nuclei preparation and shearing were performed as described previously [51] with 10 cycles sonication (30s ON/30s OFF, Bioruptor Pico, Diagenode). PU.1 was immunoprecipitated overnight at 4° C. using a polycional anti-PU.1 antibody (1:25; Cell Signaling) with the SimpleChIP Plus kit (Cell Signaling). The ratio of rs2836882 alleles in the PU.1-bound DNA was quantified in duplicate by TaqMan genotyping (assay C_2601507_20). A standard curve was generated using fixed ratios of geneblocks containing either the risk or non-risk allele (200-nt genomic sequence centred on rs2836882; Genewiz).

### PU.1 MPRA-ChIP-Seq

The MPRA vector library was transfected into TPP macrophages from six healthy donors. Assessment of PU.1 binding to SNP alleles was performed as described previously [51], with minimal sonication (to remove contaminants while minimising chromatin shearing). Immunoprecipitation was performed overnight at 4° C. using a polyclonal anti-PU.1 antibody (1:25; Cell Signaling) with the SimpleChIP Plus kit (Cell Signaling). Sequencing libraries were prepared from isolated plasmids as for MPRA and sequencing on a MiSeq (50 bp, single-end reads).

### H3K27ac ChIP-seq

TPP macrophages from two rs2836882 major allele homozygotes and two minor allele homozygotes were harvested, cross-linked, and quenched as described above. Donors were identified through the NIHR BioResource. H3K27ac ChIP-seq was performed as described previously [51] using an anti-H3K27ac antibody (1:250, Abcam) or an isotype control (1:500, rabbit IgG, Abcam). Libraries were sequenced on a HiSeq4000 (50 bp, single-end reads). Raw data were processed, QC′d, and analysed as described previously [51].

### Assays of Macrophage Effector Functions

### Flow Cytometry

Expression of myeloid markers was assessed by flow cytometry (BD LSRFortessaTMX-20). Panel: CD11b PE/Dazzle 594 (BioLegend), CD14 evolve605 (ThermoFisher), CD16 PerCP (BioLegend), CD68 FITC (BioLegend), Live/Dead Fixable Aqua Dead Cell Stain (ThermoFisher), and Fc Receptor Blocking Reagent (Miltenyi). Data were analysed using FlowJo v10 (BD Biosciences).

### Cytokine Quantification

Supernatants were collected on day 6 of TPP macrophage culture and frozen. Cytokine concentrations were quantified in duplicate via electrochemiluminescence using U-PLEX assays (Meso Scale Diagnostics).

### Phagocytosis

Phagocytosis was assessed using fluorescently-labelled Zymosan particles (Green Zymosan, Abcam) according to the manufacturer's instructions. Cells were seeded at 106 cells/well in 96-well round bottom plates. Cytochalasin D (10 μg/ml, ThermoFisher), an inhibitor of cytoskeletal rearrangement, was used as a negative control. Phagocytosis was quantified via flow cytometry, and a phagocytosis index was calculated (proportion of positive cells multiplied by their mean fluorescence intensity).

### Extracellular ROS Production

Extracellular ROS production was quantified using a Diogenes Enhanced Superoxide Detection Kit (National Diagnostics) according to the manufacturer's protocol. Cells were seeded at a density of 105 cells/well and pre-stimulated with PMA (200 ng/ml, Sigma Aldrich)

### Western Blotting

Western blotting was performed as described previously [56] using the following primary antibodies: rabbit anti-gp91phox, rabbit anti-p22phox (both Santa Cruz), rabbit anti-C17ORF62/EROS (Atlas), rabbit anti-actin (Abcam). Secondary antibody was anti-rabbit IgG-horseradish peroxidase (Cell Signaling). Chemiluminescence was recorded on a ChemiDoc Touch imager (Bio-Rad) following incubation of the membrane with ECL (ThermoFisher) or SuperSignal West Pico PLUS (ThermoFisher) reagent.

### RNA Sequencing

RNA was isolated from macrophage lysates (AllPrep DNA/RNA Micro Kit, Qiagen) and sequencing libraries prepared from 10 ng RNA using the SMARTer Stranded Total RNA-Seq Kit v2-Pico Input Mammalian (Takara) following the manufacturer's instructions. Libraries were sequenced on a NextSeq2000 (50 bp, PE reads: CRISPR-based loss-of-function, roxadustat and PD-0325901 experiments) or a NovaSeq6000 (100 bp, PE reads: overexpression experiments). Reads were trimmed using Trim Galore (Phred score 24), filtered to remove reads <20 bp, and ribosomal reads were removed using the BBSplit function of BBMap (BBMap, sourceforge.net/projects/bbmap/) with the Human ribosomal DNA complete repeating unit (GenBank: U13369.1). Reads were aligned to the human genome (hg38) using HISAT2 and converted to BAM files, sorted and indexed using SAMtools Gene read counts were obtained with the featureCounts program from Rsubread using the GTF annotation file for human genome build GRCh38 (version 102). Differential expression analysis was performed in R using the limma package [60] with the voom transformation and including donor as a covariate.

### Gene Set Enrichment Analysis

GSEA was performed using the fGSEA package in R. Gene sets were either obtained from Gene Ontology Biological Pathways (downloaded from MSigDB), experimentally-derived based on differential expression analysis, or sourced from published literature7. Pathways shown in FIGS. 2-5 are: GO: 0002274, GO: 0042116, GO: 0097529, GO: 0006909, GO: 0071706, GO: 0032732, GO: 0032755, GO: 0032757, GO: 2000379, GO: 0009060, GO: 0006119, and GO: 0045649. Statistical significance was calculated using the adaptive multilevel split Monte Carlo method.

### In Vitro Transcription

The cDNA sequence for ETS2 (NM005329.5) preceded by a Kozak sequence was synthesised and cloned into a TOPO vector. This was linearised and a PCR amplicon of the ETS2 gene generated, adding a T7 promoter and an AG initiation sequence (Phusion, NEB): Fw primer: GCTAATACGACTCACTATAAGGACAGGCCACCATGAATGATTTOGGAATC, Rv primer: TCAGTCCTCCGTGTCGG). A reverse complement (control) amplicon was also generated: Fw primer: GCTAATACGACTCACTATAAGGACAGGCCACCTCAGTCCTCCGTGTCGG, Rv primer: GCCACCATGAATGATTTOGGAATC). These amplicons were used as templates for in vitro transcription using the HiScribe T7 mRNA Kit with CleanCap® Reagent AG kit (NEB) according to the manufacturer's instructions, but with substitution of N1-methyl-pseudouridine for uridine and methylcytidine for cytidine (both Stratech) to minimise non-specific cellular activation by the transfected mRNA. mRNA was purified using a MEGAclear Transcription Clean-Up Kit (ThermoFisher) and polyadenylated using an E. coli Poly(A) Polymerase (NEB) before further clean-up (MEGAclear), quantification and analysis of product size (NorthernMax®-Gly gel, ThermoFisher). For optimising overexpression conditions, GFP mRNA was produced using the same method: Fw primer GCTAATACGACTCACTATAAGGACAGGCCACCATGGTGAGCAAGGGCGAG, Rv primer TTACTTGTACAGCTCGTCCATGC).

mRNA Overexpression

Lipofectamine MessengerMAX (ThermoFisher) was diluted in Opti-MEM (1:75 v/V), vortexed and incubated at room temperature for 10 minutes. IVT mRNA was then diluted in a fixed volume of Opti-MEM (112.5 μl per transfection), mixed with an equal volume of diluted Lipofectamine MessengerMAX and incubated for a further 5 minutes at room temperature. The transfection mix was then added dropwise to 2.5×106 M0 macrophages (pre-cultured for 6 days in a 6-well plate in antibiotic-free RPMI1640 macrophage media containing M-CSF (50 ng/ml, Peprotech)—with media change on d3). For GFP overexpression, cells were detached using Accutase 18 hours after transfection and GFP expression was measured by flow cytometry. For ETS2/control overexpression, either 250 ng or 500 ng mRNA was transfected and low dose LPS (0.5 ng/ml) was added 18 hours after transfection, and cells detached using Accutase 6 hours later (n=8 donors). Representative ETS2 expression in untransfected macrophages obtained from previous data (GSE193336).

### SNPsea

Pathway analysis of 241 IBD-associated GWAS hits [7] was performed using SNPsea [36]. In brief, linkage intervals were defined for every lead SNP based on the furthest correlated SNPs (r2>0.5 in 1000 Genomes, EUR population) and extended to the nearest recombination hotspots with recombination rate >3 cM/Mb. If no genes were present in this region, the linkage interval was extended up- and down-stream by 500 kb. Genes within linkage intervals were tested for enrichment within 7,660 pathways, comprising 7,658 Gene Ontology Biological Pathways and 2 lists of ETS2-regulated genes (either those significantly downregulated following ETS2 disruption with gRNA1 or those significantly upregulated following ETS2 overexpression, based on a consensus list obtained from differential expression analysis including all samples and using donor and mRNA quantity as covariates). The analysis was performed using a single score mode: assuming that only one gene per linkage interval is associated with the pathway. A null distribution of scores for each pathway was performed by sampling random SNP sets matched on the number of linked genes (5,000,000 iterations). A permutation P-value was calculated by comparing the enrichment of the IBD-associated gene list with the null distribution. Gene sets relating to the following IBD-associated pathways were extracted for comparison: NOD2 signalling (GO: 0032495), Integrin signalling (GO: 0033627, GO: 0033622), TNFα signalling (GO: 0033209, GO: 0034612), Intestinal epithelium (GO: 0060729, GO: 0030277), Th17 cells (GO: 0072539, GO: 0072538, GO: 2000318), T cell activation (GO: 0046631, GO: 0002827), IL-10 signalling (GO: 0032613, GO: 0032733), and autophagy (GO: 0061919, GO: 0010506, GO: 0010508, GO: 1905037, GO: 0010507).

### ETS2 Co-Expression

Genes co-expressed with ETS2 across 64 human monocyte-derived macrophage polarisation conditions (normalised data from GSE47189) were identified using the rcorr function in the Hmisc package in R.

### ¹³C-Glucose GC-MS

ETS2-edited or unedited TPP macrophages were generated in triplicate for each donor and on day 6, media was removed, cells washed with PBS, and new media with labelled glucose was added. Labelled media: RPMI1640 Medium, no glucose (ThermoFisher); 10% FBS (ThermoFisher); GlutaMAX (ThermoFisher); ¹³C-labelled glucose (Cambridge Isotype Laboratories). After 24 hours—a time point selected from a time-course to establish steady-state conditions—supernatants were snap-frozen and macrophages detached by scraping. Macrophages were washed three times with ice-cold PBS, counted, re-suspended in 600 μl ice-cold chloroform:methanol (2:1, v/V) and sonicated in a waterbath (3×8 mins). All extraction steps were performed at 4° C. as previously described [62]. Samples were analysed in an Agilent 7890B-7000C GC-MS system. Spitless injection (injection temperature 270° C.) onto a DB-5 MS (Agilent) was used, using helium as the carrier gas, in electron ionization mode. The initial oven temperature was 70° C. (2 min), followed by temperature gradients to 295° C. at 12.5° C. per min and to 320° C. at 25° C. per min (held for 3 min). Scan range was m/z 50-550. Data analysis was performed using in-house software MANIC (version 3.0), based on the software package GAVIN [63]. Label incorporation was calculated by subtracting the natural abundance of stable isotopes from the observed amounts. Total metabolite abundance was normalised to the internal standard (scyllo-inositol [62]).

### Roxadustat

ETS2-edited or unedited TPP macrophages were generated as described previously. On day 5 of culture, cells were detached (Accutase) and re-plated at a density of 10⁵ cells/well in 96-well round bottom plates in TPP media containing Roxadustat (FG-4592, 30 μM). After 12 hours, cells were harvested for functional assays and RNA-seq as described.

### CUT&RUN

Pre-cultured TPP macrophages were harvested and processed immediately using the CUT&RUN Assay kit (Cell Signaling) according to the manufacturer's instructions but omitting the use of ConA-coated beads. In brief, 5×10⁶ cells per reaction were pelleted, washed, and resuspended in Antibody Binding buffer. Cells were incubated with antibodies: anti-ETS2 (1:100, ThermoFisher) or IgG control (1:20, Cell Signaling) for 2 h at 4° C. After washing in Digitonin Buffer, cells were incubated with pA/G-MNase for 1 h at 4° C. Cells were washed twice in Digitonin Buffer, resuspended in the same buffer and cooled for 5 minutes on ice. Calcium chloride was added to activate pA/G-MNase digestion (30 min, 4° C.) before the reaction was stopped and cells incubated at 37° C. for 10 min to release cleaved chromatin fragments. Supernatants were collected by centrifugation and DNA extracted using spin columns (Cell Signaling). Library preparation was performed using a protocols.

IO protocol (dx.doi.org/10.17504/protocols.io.bagaibse) with the NEBNext Ultra II DNA Library Prep Kit. Size selection was performed using AMPure XP beads (Beckman Coulter) and fragment sizes assessed using an Agilent 2100 Bioanalyzer (High Sensitivity DNA kit). Equimolar pools of indexed libraries were sequenced on a NovaSeq6000 (100 bp PE reads). Raw data were analysed using guidelines from the Henikoff lab [64]. Briefly, paired-end reads were trimmed using Trim Galore and aligned to the human genome (GRCh37/hg19) using Bowtie2. BAM files were sorted, merged (technical and, where indicated, biological replicates), re-sorted and indexed using SAMtools. Picard was used to mark unmapped reads and SAMtools to remove these reads, re-sort and re-index. Bigwig files were created using the deepTools bamCoverage function. Peaks were called using MACS2. The Irreproducible Discovery Rate was calculated across replicates (MACS2 peaks called using FDR q<0.05) using a 0.01 cutoff with the idr package in R. Enrichment of an ETS2 binding motif in consensus IDR peaks was calculated using TFmotifView [65] using global genomic controls. Overlap between consensus IDR peaks and the core promoter (−250 bp to +35 bp from TSS) and/or putative cis-regulatory elements of ETS2-regulated genes was assessed using lists of differentially-expressed genes following ETS2 disruption with gRNA1 or ETS2 overexpression (based on a consensus across mRNA doses, as described earlier). Putative cis-regulatory elements were defined as shared interactions (CHICAGO score >5) in monocyte, M0 and M1 macrophage samples from publicly-available promoter-capture Hi-C data [13].

### ATAC-Seq

ATAC-seq in TPP macrophages was performed using the Omni-ATAC protocol with the following modifications: cell number was increased to 75,000 cells, cell lysis time was increased to 5 minutes; volume of Tn5 transposase in the transposition mixture was doubled; duration of the transposition step was extended to 40 minutes. Amplified libraries were cleaned using AMPure XP beads (Beckman Coulter) and sequenced on a NovaSeq6000 (100 bp PE reads). Data were processed as described previously [67].

### Chr21q22 Disease Datasets

Publicly-available raw RNA-seq data from the affected tissues of chr21q22-associated diseases (and controls from the same experiment) were downloaded from GEO: IBD macrophages (GSE123141), primary sclerosing cholangitis liver (GSE159676), ankylosing spondylitis synovium (GSE41038). Reads were trimmed, filtered, and aligned as described earlier. For each disease dataset, a ranked list of genes was obtained by differential expression analysis between cases and controls using limma with voom transformation. For IBD macrophages, only IBD samples with active disease were included. fGSEA using ETS2-regulated gene lists was performed as described.

### LINCS Signatures

31,027 lists of down-regulated genes following exposure of a cell line to a small molecule were obtained from the NIH LINCS database (downloaded in January 2021). These were used as gene sets for fGSEA (as described) with a ranked list of genes obtained by differential expression analysis between ETS2-edited and unedited TPP macrophages (gRNA1)—using limma with voom transformation and donor as a covariate. Drug classes for gene sets with FDR P<0.05 were manually assigned based on known mechanisms of action.

### PD-0325901

TPP macrophages were generated as described previously. On day 4 of culture, PD-0325901 (0.5 μM, Sigma) or vehicle (DMSO) were added. Cells were harvested on day 6 and RNA was extracted and sequenced as described.

### Colonic Biopsies

During colonoscopy, intestinal mucosal biopsies (6 per donor) were collected from 10 IBD patients (7 ulcerative colitis, 3 Crohn's disease). All had endoscopically active disease and were not receiving immunosuppressive or biologic therapies. All biopsies were collected from a single inflamed site. All patients provided written informed consent. Ethical approval was provided by the London-Brent Regional Ethics Committee (REC: 21/LO/0682). Biopsies were collected into Opti-MEM and within 1 hour were weighed and placed in pairs onto a transwell insert (ThermoFisher)—designed to create an air-liquid interface70—in a 24-well plate. Each well contained 1 ml media and was supplemented with either DMSO (vehicle control), PD-0325901 (0.5 μM) or infliximab (10 g/ml; MSD). Media: Opti-MEM I (Gibco); GlutaMAX (ThermoFisher): 10% FBS (ThermoFisher), MEM Non-Essential Amino Acids (ThermoFisher); 1% sodium pyruvate (ThermoFisher); 1% penicillin/streptomycin (ThermoFisher); 500 g/ml gentamicin (Merck). After 18 hours, supernatants and biopsies were snap frozen. Supernatant cytokine concentrations were quantified using LEGENDplex Human Inflammation Panel (Biolegend). RNA was extracted from biopsies and libraries prepared as described earlier (n=9, RNA from one donor was too degraded). Sequencing was performed on a NovaSeq 6000 (100 bp, PE reads). Data were processed as described earlier and GSVA was performed for ETS2-regulated genes and biopsy-derived signatures of IBD-associated inflammation [48].

### Statistical Methodology

Statistical methods used in MPRA analysis, fGSEA, and SNPsea are described above. For other analyses, comparison of continuous variables between paired samples in two groups was performed using a Wilcoxon matched-pairs test for non-parametric data or a paired t-test for parametric data. Comparison against a hypothetical value was performed using a Wilcoxon signed-rank test for non-parametric data or one sample t-test for parametric data. A Shapiro-Wilk test was used to confirm normality. Two-tailed tests were used as standard unless a specific hypothesis was being tested. Sample sizes are provided in respective sections.

### Example 11—ETS2-Driven Inflammatory Macrophages are Present in all of the Chr21q22-Associated Autoinflammatory Diseases

The inventors have shown that overexpressing ETS2 in resting macrophages produced a transcriptional state that closely resembled the phenotype of intestinal macrophages in Crohn's disease. The inventors also broadened this analysis to show that this also applies to macrophages from other chr21q22-associated diseases as well. To do this, publicly reported macrophage signatures from a range of different diseases were identified. Importantly, these represented true disease signatures derived from human macrophages from patients with the respective conditions (rather than responses to in vitro stimulation). The signatures are shown in Table 1.

TABLE 1
Macrophage-derived disease signatures. DEGs (up), differentially-expressed genes
(upregulat-ed); TAMs, tumour-associated macrophages, BAL, bronchoalveolar lavage.

| | Tissue | | | | |
|---|---|---|---|---|---|
| Disease | source | PMID | Source (in paper) | Method | Category |
| Crohn's disease | Intestine | 30670762 | Cluster E and F | scRNAseq | Inflammatory |
| | | | markers | | disease |
| | | | | | (chr21q22- |
| | | | | | associated) |
| Takayasu | Blood | 34671607 | DEGs (up) in | scRNAseq | Inflammatory |
| arteritis | | | CD14+ monocytes | | disease |
| | | | (vs controls) | | (chr21q22- |
| | | | | | associated) |
| Ankylosing | Blood | 37388915 | DEGs (up) in | RNAseq | Inflammatory |
| spondylitis | | | CD14+ monocytes | (bulk) | disease |
| | | | | | (chr21q22- |
| | | | | | associated) |
| Ulcerative colitis | Intestine | 31348891 | DEGs (up) in | scRNAseq | Inflammatory |
| | | | m. monocyte | | disease |
| | | | subgroups (vs | | (chr21q22- |
| | | | controls) | | associated) |
| Influenza A | Upper airway | 34618691 | DEGs (up) in | scRNAseq | Viral infection |
| | | | macrophages (vs | | |
| | | | controls) | | |
| COVID-19 | Upper airway | 34618691 | DEGs (up) in | scRNAseq | Viral infection |
| | | | macrophages (vs | | |
| | | | controls) | | |
| Tuberculosis | Broncho- | 37470432 | DEGs (up) in | scRNAseq | Bacterial infection |
| | alveolar | | macrophages | | |
| | lavage | | (active TB vs | | |
| | | | controls,) | | |
| Bacterial sepsis | Blood | 32066974 | DEGs (up) in MS1 | scRNAseq | Bacterial infection |
| | | | vs all monocytes | | |
| Tumour- | Tumour | 34331874 | DEGs (up) in | scRNAseq | Cancer |
| associated | (lung, colon, | | IL4I1+ TAMs | | |
| (multiple | liver, breast, | | (cluster #6) | | |
| cancers) | stomach, and | | | | |
| | pancreas) | | | | |
| Tumour- | Tumour | 30930117 | DEGs (up) in | scRNAseq | Cancer |
| associated | | | endometrial TAMs | | |
| (endometrial) | | | vs endometrial | | |
| | | | tissue resident | | |
| | | | macs | | |
| Atherosclerosis | Atherosclerotic | 36190844 | Foamy | scRNAseq | Atherosclerosis |
| | plaques | | macrophage | | |
| | | | marker genes | | |

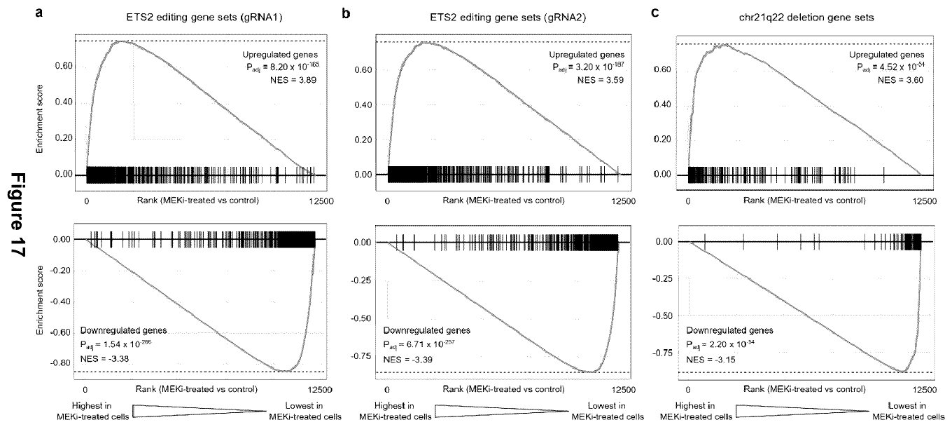

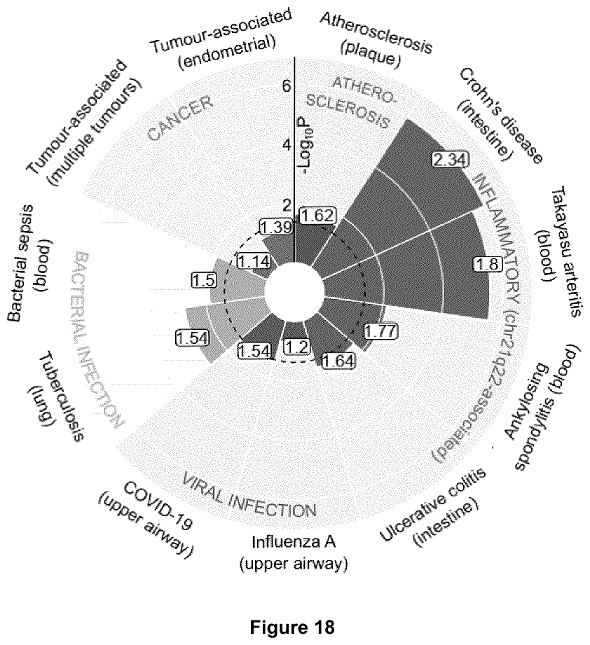

These signatures were used as gene sets for fGSEA to assess whether ETS2 overexpression induced similar enrichment across all diseases. The results are shown FIG. 18. They demonstrate that ETS2 overexpression does not simply cause baseline activation, but rather induces genes that are characteristic of chronic inflammatory disease and, to a lesser extent, responses to bacterial infection. In contrast, no significant enrichment was observed for tumour-associated macrophage signatures or macrophages during certain viral infections (e.g. influenza A) (FIG. 18).

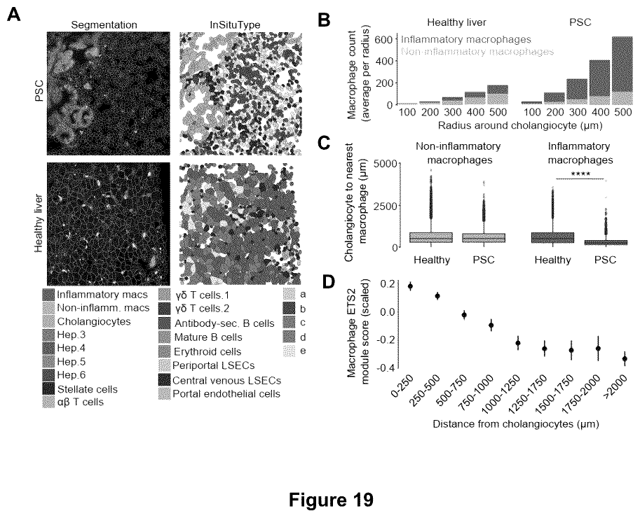

The one missing chr21q22-associated disease from this analysis was primary sclerosing cholangitis (PSC) since no suitable datasets were available. To address this, the inventors performed spatial transcriptomics on fixed liver tissue from PSC and unaffected controls revealed increased numbers of inflammatory macrophages in the liver in PSC, which were in close proximity to cholangiocytes—the principal target of pathology (FIG. 18). Moreover, ETS2-regulated genes were most highly expressed in the macrophages nearest to PSC cholangiocytes-consistent with a role in disease pathogenesis (FIG. 19).

Together, the data demonstrates that ETS2-driven inflammatory macrophages are present in all of the chr21q22-associated autoinflammatory diseases, and therefore that this provides a novel therapeutic target.

### Methods

Spatial transcriptomics: 5 μm FFPE sections were cut from two PSC liver explants and two controls (healthy liver adjacent to tumour metastases), baked overnight at 60° C. and prepared for CosMx according to manufacturer's instructions using 15 min target retrieval and 30 min protease digestion. Tissue samples were obtained via Tissue Access for Patient Benefit (TAP-B, part of the UCL-RFH Biobank) under research ethics approval: 16/WA/0289 (Wales Research Ethics Committee 4). One case and one control were included on each slide. The Human Universal Cell Characterization core panel (960 genes) was used, supplemented with 8 additional genes to improve identification of cells of interest: CD1D, EREG, ETS2, FCN1, GOS2, LYVE1, MAP2K1, MT1G. Segmentation was performed using the CosMx Human Universal Cell Segmentation Kit (RNA), Human IO PanCK/CD45 Kit (RNA) and Human CD68 Marker, Ch5 (RNA). FOVs were tiled across all available regions (221 control, 378 PSC) and cyclic fluorescent in situ hybridization (FISH) was performed using the CosMx SMI (Nanostring). Data were pre-processed on the AtoMx Spatial Informatics Platform—with images segmented to obtain cell boundaries, transcripts assigned to single cells, and a transcript by cell count matrix obtained. Expression matrices, transcript coordinates, polygon coordinates, FOV coordinates, and cell metadata were exported, and QC, normalisation and cell-typing were performed using InsituType—an R package developed to extract all the information available in every cell's expression profile. A semi-supervised strategy was used to phenotype cells, incorporating the Liver Human Cell Atlas reference matrix. Spatial analysis of macrophage phenotypes was performed according to proximity from cholangiocytes (anchor cell type). Radius and Nearest Neighbour analysis were performed using PhenoptR (https://akoyabio.github.io/phenoptr/) with macrophage distribution from cholangiocytes binned in 100 μm increments up to 500 μm. Nearest neighbour analysis was performed to determine the distance from cholangiocytes to the nearest inflammatory and non-inflammatory macrophage and vice versa.

Gene set enrichment analysis (GSEA): GSEA was performed using fGSEA in R with differentially-expressed gene lists ranked by t-statistic. Gene sets were obtained from Gene Ontology Biological Pathways (MSigDB), experimentally-derived based on differential expression analysis, or sourced from published literature. Specific details of disease macrophage signatures are listed in Table 1.

### SEQUENCES

| Sequence number | Description/Sequence |
|---|---|
| SEQ ID NO: 1 | chr21q22 enhancer sequence, hg19: chr21: 40466236-40466677 |
| SEQ ID NO: 2 | ETS2 mRNA sequence (NM005239.6) |
| SEQ ID NO: 3 | ETS2 mRNA sequence (NM_001256295.2) |
| SEQ ID NO: 4 | ETS2 amino acid sequence (Uniprot: P15036) |
| SEQ ID NO: 5 | ETS2 genomic nucleic acid sequence, chr21: 40176507-40197479 |
| SEQ ID NO: 6 | chr21q22 5′ gRNA, CCUGGCUGCCUCGCGUUUCC |
| SEQ ID NO: 7 | chr21q22 3′ gRNA, CCUCGUCCAACAGAGAGCAA |
| SEQ ID NO: 8 | ETS2 gRNA1, CAGACACAGAAUUACCCCAA |
| SEQ ID NO: 9 | ETS2 gRNA2, UUGCUGCACGGGGUUAACAA |
| SEQ ID NO: 10 | chr21q22_Fw primer, GGTGGGGAGAGTTCCAAAGG |
| SEQ ID NO: 11 | chr21q22_Rv primer, TCACCCTTCACCTCTTTGCT |
| SEQ ID NO: 12 | ETS2_g1_Fw, TCCTGAAGGTCCCATGAAAG |
| SEQ ID NO: 13 | ETS2_g1_Rv, TCATTATGGCTCTGGGGTTC |
| SEQ ID NO: 14 | ETS2_g2_Fw, GCGGCACATTCATATCACAC |
| SEQ ID NO: 15 | ETS2_g2_Rv, GCAGAATACCCCAAGCAAAA |
| SEQ ID NO: 16 | 16-nt universal primer site ACTGGCCGCTTCACTG |
| SEQ ID NO: 17 | KpnI and XbaI restriction sites (TGGACCTCTAGA) |
| SEQ ID NO: 18 | 17-nt universal primer site AGATCGGAAGAGCGTCG |
| SEQ ID NO: 19 | ETS2 Fw primer: |
| | GCTAATACGACTCACTATAAGGACAGGCCACCATGAATGATTTOGGAATO |
| SEQ ID NO: 20 | ETS2 Rv primer: GCCACCATGAATGATTTCGGAATC |
| SEQ ID NO: 21 | GFP Fw primer: |
| | GCTAATACGACTCACTATAAGGACAGGCCACCATGGTGAGCAAGGGCGAG |
| SEQ ID NO: 22 | GFP Rv primer: TTACTTGTACAGCTCGTCCATGC |

### EQUIVALENTS AND SCOPE

Those skilled in the art will appreciate that the present invention is defined by the appended claims and not by the Examples or other description of certain embodiments included herein.

Similarly, the singular forms “a”, “an”, and “the” include plural referents unless the context clearly dictates otherwise.

Unless defined otherwise above, all technical and scientific terms used herein have the same meaning as commonly understood by one of ordinary skill in the art to which this invention belongs. Any methods and materials similar or equivalent to those described herein can also be used in the practice or testing of the present invention. Generally, nomenclatures used in connection with, and techniques of, cell and tissue culture, molecular biology, immunology, genetics and protein and nucleic acid chemistry described herein are those well-known and commonly used in the art, or according to manufacturer's specifications.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

The invention is also described in the following numbered embodiments.
1. A method of treating or preventing a disease in a subject, the method comprising administering an Erythroblast Transformation Specific proto-oncogene 2 (ETS2) inhibitor to the subject.
2. A method of treating or preventing a disease in a subject, the method comprising administering an inhibitor of the chr21q22 enhancer (SEQ ID NO:1) to the subject.
3. A method of treating or preventing a disease in a subject, the method comprising modifying or deleting a portion of the chr21q22 enhancer (SEQ ID NO:1) to the subject, optionally wherein the entire chr21q22 enhancer (SEQ ID NO: 1) is deleted.
4. A method of reducing macrophage activation by contacting said macrophage with an inhibitor of Erythroblast Transformation Specific proto-oncogene 2 (ETS2).
5. A method of reducing macrophage activation by contacting said macrophage with an inhibitor of the chr21q22 enhancer (SEQ ID NO:1).
6. A method of reducing macrophage activation by modifying or deleting a portion of the chr21q22 enhancer (SEQ ID NO:1), optionally wherein the entire chr21q22 enhancer (SEQ ID NO:1) is deleted.
7. A method of reducing proinflammatory cytokine production by contacting a macrophage with an inhibitor of Erythroblast Transformation Specific proto-oncogene 2 (ETS2).
8. A method of reducing proinflammatory cytokine production by contacting a macrophage with an inhibitor of the chr21q22 enhancer (SEQ ID NO:1).
9. A method of reducing proinflammatory cytokine production by modifying or deleting a portion of the chr21q22 enhancer (SEQ ID NO: 1), optionally wherein the entire chr21q22 enhancer (SEQ ID NO: 1) is deleted.
10. A method of reducing reactive oxygen species (ROS) production by contacting a macrophage with an inhibitor of Erythroblast Transformation Specific proto-oncogene 2 (ETS2).
11. A method of reducing reactive oxygen species (ROS) production by contacting a macrophage with an inhibitor of the chr21q22 enhancer (SEQ ID NO:1).
12. A method of reducing reactive oxygen species (ROS) production by modifying or deleting a portion of the chr21q22 enhancer (SEQ ID NO:1), optionally wherein the entire chr21q22 enhancer (SEQ ID NO:1) is deleted.
13. A method of reducing macrophage phagocytosis by contacting said macrophage with an inhibitor of Erythroblast Transformation Specific proto-oncogene 2 (ETS2).
14. A method of reducing macrophage phagocytosis by contacting said macrophage with an inhibitor of the chr21q22 enhancer (SEQ ID NO:1).
15. A method of reducing macrophage phagocytosis by modifying or deleting a portion of the chr21q22 enhancer (SEQ ID NO:1), optionally wherein the entire chr21q22 enhancer (SEQ ID NO: 1) is deleted.
16. A method of reducing macrophage migration by contacting said macrophage with an inhibitor of Erythroblast Transformation Specific proto-oncogene 2 (ETS2).
17. A method of reducing macrophage migration by contacting said macrophage with an inhibitor of the chr21q22 enhancer (SEQ ID NO:1).
18. A method of reducing macrophage migration by modifying or deleting a portion of the chr21q22 enhancer (SEQ ID NO:1), optionally wherein the entire chr21q22 enhancer (SEQ ID NO:1) is deleted.
19. The method according to any one of embodiments 4 to 6, wherein a reduction in macrophage activation is indicated by one or more of a reduction in cellular activation, a reduction in proinflammatory cytokine production, metabolic reprogramming, a reduction in reactive oxygen species production, and/or a reduction in macrophage migration.
20. The method according to any one of embodiments 7 to 9, wherein a reduction in proinflammatory cytokine production comprises a reduction in the level of one or more of TNFα, IL-1, IL-6, IL-8, IL-12, IL-23 and/or IL-18.
21. The method according to any one of embodiments 10 to 12, wherein a reduction in reactive oxygen species (ROS) production comprises a reduction in the level of one or more of hydrogen peroxide, hydroxyl radical, superoxide anion and/or singlet oxygen.
22. The method according to any one of embodiments 13 to 15, wherein a reduction in macrophage phagocytosis is indicated by a reduction in uptake of particulate matter (for example bacteria) by macrophages, quantified by an in vitro or in vivo assay.
23. The method according to any one of embodiments 16 to 18, wherein a reduction in macrophage migration is indicated by and reduction in in vitro measures of macrophage motility (for example quantified by a chemotaxis assay) and/or a reduction in the total number of macrophages that accumulate at sites of inflammation in vivo.
24. A method of treating a disease in a subject in need thereof, the method comprising administering to the subject an Erythroblast Transformation Specific proto-oncogene 2 (ETS2) inhibitor, thereby reducing macrophage activation in the subject.
25. A method of treating a disease in a subject in need thereof, the method comprising administering to the subject a chr21q22 enhancer (SEQ ID NO:1) inhibitor, thereby reducing macrophage activation in the subject.
26. A method of treating a disease in a subject in need thereof, the method comprising modifying or deleting a portion of the chr21q22 enhancer (SEQ ID NO:1), thereby reducing macrophage activation in the subject.
27. The method of treating a disease in a subject according to any one of embodiments 1 to 3 or 24 to 26, wherein the subject has an inflammatory disease and/or autoimmune disease.
28. The method of treating a disease in a subject according to embodiment 27, wherein the inflammatory disease is selected from the list consisting of: Crohn's disease, ulcerative colitis, inflammatory bowel disease (Crohn's disease and/or ulcerative colitis), ankylosing spondylitis, primary sclerosing cholangitis, Takayasu arteritis and rheumatoid arthritis.
29. An Erythroblast Transformation Specific proto-oncogene 2 (ETS2) inhibitor for use in a method of treating or preventing a disease in a subject.
30. An inhibitor of the chr21q22 enhancer (SEQ ID NO:1) for use in a method of treating or preventing a disease in a subject.
31. An Erythroblast Transformation Specific proto-oncogene 2 (ETS2) inhibitor for use in a method of reducing macrophage activation by contacting said macrophage with an inhibitor of Erythroblast Transformation Specific proto-oncogene 2 (ETS2).
32. An inhibitor of the chr21q22 enhancer (SEQ ID NO:1) for use in a method of reducing macrophage activation by contacting said macrophage with an inhibitor of the chr21q22 enhancer (SEQ ID NO:1).
33. An Erythroblast Transformation Specific proto-oncogene 2 (ETS2) inhibitor for use in a method of reducing proinflammatory cytokine production by contacting a macrophage with an inhibitor of Erythroblast Transformation Specific proto-oncogene 2 (ETS2).
34. An inhibitor of the chr21q22 enhancer (SEQ ID NO:1) for use in a method of reducing proinflammatory cytokine production by contacting a macrophage with an inhibitor of the chr21q22 enhancer (SEQ ID NO:1).
35. An Erythroblast Transformation Specific proto-oncogene 2 (ETS2) inhibitor for use in a method of reducing reactive oxygen species (ROS) production by contacting a macrophage with an inhibitor of Erythroblast Transformation Specific proto-oncogene 2 (ETS2).
36. An inhibitor of the chr21q22 enhancer (SEQ ID NO:1) for use in a method of reducing reactive oxygen species (ROS) production by contacting a macrophage with an inhibitor of the chr21q22 enhancer (SEQ ID NO:1).
37. An Erythroblast Transformation Specific proto-oncogene 2 (ETS2) inhibitor for use in a method of reducing macrophage phagocytosis by contacting said macrophage with an inhibitor of Erythroblast Transformation Specific proto-oncogene 2 (ETS2).
38. An inhibitor of the chr21q22 enhancer (SEQ ID NO:1) for use in a method of reducing macrophage phagocytosis by contacting said macrophage with an inhibitor of the chr21q22 enhancer (SEQ ID NO:1).
39. An Erythroblast Transformation Specific proto-oncogene 2 (ETS2) inhibitor for use in a method of reducing macrophage migration by contacting said macrophage with an inhibitor of Erythroblast Transformation Specific proto-oncogene 2 (ETS2).
40. An inhibitor of the chr21q22 enhancer (SEQ ID NO:1) for use in a method of reducing macrophage migration by contacting said macrophage with an inhibitor of the chr21q22 enhancer (SEQ ID NO:1).
41. The Erythroblast Transformation Specific proto-oncogene 2 (ETS2) or chr21q22 enhancer inhibitor for use according to any one of embodiments 31 or 32, wherein a reduction in macrophage activation is indicated by one or more of a reduction in cellular activation, a reduction in proinflammatory cytokine production, metabolic reprogramming, a reduction in reactive oxygen species production, and/or a reduction in macrophage migration.
42. The Erythroblast Transformation Specific proto-oncogene 2 (ETS2) or chr21q22 enhancer inhibitor for use according to any one of embodiments 33 or 34, wherein a reduction in proinflammatory cytokine production comprises a reduction in the level of one or more of TNFα, IL-1, IL-6, IL-8, IL-12, IL-23 and/or IL-18.
43. The Erythroblast Transformation Specific proto-oncogene 2 (ETS2) or chr21q22 enhancer inhibitor for use according to any one of embodiments 35 or 36, wherein a reduction in reactive oxygen species (ROS) production comprises a reduction in the level of one or more of hydrogen peroxide, hydroxyl radical, superoxide anion and/or singlet oxygen.
44. The Erythroblast Transformation Specific proto-oncogene 2 (ETS2) or chr21q22 enhancer inhibitor for use according to any one of embodiments 37 or 38, wherein a reduction in macrophage phagocytosis is indicated by a reduction in uptake of particulate matter (for example bacteria) by macrophages, quantified by an in vitro or in vivo assay.
45. The Erythroblast Transformation Specific proto-oncogene 2 (ETS2) or chr21q22 enhancer inhibitor for use according to any one of embodiments 39 or 40, wherein a reduction in macrophage migration is indicated by a reduction in in vitro measures of macrophage motility (for example quantified by a chemotaxis assay) and/or a reduction in the total number of macrophages that accumulate at sites of inflammation in vivo.
46. An Erythroblast Transformation Specific proto-oncogene 2 (ETS2) inhibitor for use in a method of treating a disease in a subject in need thereof, the method comprising administering to the subject an Erythroblast Transformation Specific proto-oncogene 2 (ETS2) inhibitor, thereby reducing macrophage activation in the subject.
47. An inhibitor of the chr21q22 enhancer (SEQ ID NO:1) for use in a method of treating a disease in a subject in need thereof, the method comprising administering to the subject a chr21q22 enhancer (SEQ ID NO:1) inhibitor, thereby reducing macrophage activation in the subject.
48. The Erythroblast Transformation Specific proto-oncogene 2 (ETS2) or chr21q22 enhancer inhibitor for use according to any one of embodiments 29 to 47, wherein the subject has an inflammatory disease and/or autoimmune disease.
49. The Erythroblast Transformation Specific proto-oncogene 2 (ETS2) or chr21q22 enhancer inhibitor for use according to embodiment 48, wherein the inflammatory disease is selected from the list consisting of: Crohn's disease, ulcerative colitis, inflammatory bowel disease (Crohn's disease and/or ulcerative colitis), ankylosing spondylitis, primary sclerosing cholangitis, Takayasu arteritis and rheumatoid arthritis.
50. The method, Erythroblast Transformation Specific proto-oncogene 2 (ETS2) or chr21q22 enhancer inhibitor for use according to any one of embodiments 1 to 49, wherein the ETS2 inhibitor or chr21q22 enhancer inhibitor comprises an antisense nucleic acid molecule, a small interfering RNA (siRNA), or a short hairpin RNA (shRNA).
51. The method, Erythroblast Transformation Specific proto-oncogene 2 (ETS2) or chr21q22 enhancer inhibitor for use according to any one of embodiments 1 to 49, wherein the ETS2 inhibitor or chr21q22 enhancer inhibitor comprises a small molecule, inhibitory peptide, an antibody or a nanobody.
52. The method, Erythroblast Transformation Specific proto-oncogene 2 (ETS2) or chr21q22 enhancer inhibitor for use according to embodiment 52, wherein the antibody is a monoclonal antibody that targets surface markers specific to ETS2-positive macrophages to inhibit effector function and/or induce destruction.
53. The method, Erythroblast Transformation Specific proto-oncogene 2 (ETS2) or chr21q22 enhancer inhibitor for use according to embodiment 52, wherein the nanobody targets surface markers specific to ETS2-positive macrophages to inhibit effector function and/or induce destruction.
54. The method, Erythroblast Transformation Specific proto-oncogene 2 (ETS2) or chr21q22 enhancer inhibitor for use according to any one of embodiments 1 to 49, wherein the ETS2 inhibitor or chr21q22 enhancer inhibitor comprises a proteolysis-targeting chimera (PROTAC) that targets ETS2 for degradation, a molecular glue that targets ETS2 for degradation, an antibody-drug conjugate, an antibody-RNA conjugate, an esterase-sensitive motif-conjugated drug or a nanobody-drug conjugate.
55. The method, Erythroblast Transformation Specific proto-oncogene 2 (ETS2) or chr21q22 enhancer inhibitor for use according to embodiment 54, wherein the antibody-drug conjugate comprises an antigen binding molecule that specifically binds a macrophage marker, optionally wherein the antigen binding molecule is an antibody.
56. The method, Erythroblast Transformation Specific proto-oncogene 2 (ETS2) or chr21q22 enhancer inhibitor for use according to embodiment 54, wherein the antibody-drug conjugate comprises an anti-CD163 antigen binding molecule, optionally wherein the antigen binding molecule is an antibody.
57. The method, Erythroblast Transformation Specific proto-oncogene 2 (ETS2) or chr21q22 enhancer inhibitor for use according to embodiment 54, wherein the antibody-drug conjugate comprises an anti-CD209 (DC-SIGN) or an anti-CD206 (MRC1) antigen binding molecule, optionally wherein the antigen binding molecule is an antibody.
58. The method, Erythroblast Transformation Specific proto-oncogene 2 (ETS2) or chr21q22 enhancer inhibitor for use according to embodiment 54 to 57, wherein the antibody-drug conjugate comprises a MEK inhibitor, optionally wherein the MEK inhibitor is selected from the list consisting of Selumetinib, Trametinib and Cobimetinib, preferably wherein the MEK inhibitor is Selumetinib.
59. The method, Erythroblast Transformation Specific proto-oncogene 2 (ETS2) or chr21q22 enhancer inhibitor for use according to any one of embodiments 54 to 59, wherein the antibody-drug conjugate comprises a cleavable linker, optionally wherein the cleavable linker is glucuronide-based (e.g. GlyPro).
60. The method, Erythroblast Transformation Specific proto-oncogene 2 (ETS2) or chr21q22 enhancer inhibitor for use according to any one of embodiments 54 to 59, wherein the antibody-drug conjugate comprises a non-cleavable linker, optionally wherein the non-cleavable linker is maleimide-PEG3-based.
61. The method, Erythroblast Transformation Specific proto-oncogene 2 (ETS2) or chr21q22 enhancer inhibitor for use according to any one of embodiments 54 to 60, wherein the antibody-drug conjugate has an antibody: drug ratio of at least 1:2, at least 1:4, at least 1:6, at least 1:8 or at least 1:10.
62. The method, Erythroblast Transformation Specific proto-oncogene 2 (ETS2) or chr21q22 enhancer inhibitor for use according to embodiment 54, wherein the esterase-sensitive motif-conjugated drug comprises cyclopentyl L-leucinate or cyclopentyl(S)-2-amino-2-cyclohexylacetate.
63. The method, Erythroblast Transformation Specific proto-oncogene 2 (ETS2) or chr21q22 enhancer inhibitor for use according to embodiment 54 or 62, wherein the esterase-sensitive motif-conjugated drug comprises a MEK inhibitor, optionally wherein the MEK inhibitor is selected from the list consisting of Selumetinib, Trametinib and Cobimetinib.
64. The method, Erythroblast Transformation Specific proto-oncogene 2 (ETS2) or chr21q22 enhancer inhibitor for use according to embodiment 54 or 62 to 63, wherein the esterase-sensitive motif-conjugated drug comprises a linker.
65. The method, Erythroblast Transformation Specific proto-oncogene 2 (ETS2) or chr21q22 enhancer inhibitor for use according to embodiment 64, wherein the linker is one or two carbons in length.
66. The method, Erythroblast Transformation Specific proto-oncogene 2 (ETS2) or chr21q22 enhancer inhibitor for use according to any one of embodiments 1 to 49, wherein the ETS2 inhibitor or chr21q22 enhancer inhibitor comprises an agent that disrupts the function of co-transcriptional activators (e.g. BRD4 or CDKs).
67. The method, Erythroblast Transformation Specific proto-oncogene 2 (ETS2) or chr21q22 enhancer inhibitor for use according to any one of embodiments 1 to 49, wherein the ETS2 inhibitor or chr21q22 enhancer inhibitor comprises an agent that targets chromatin regulators (e.g. readers, writers and erasers of chromatin modifications).
68. The method, Erythroblast Transformation Specific proto-oncogene 2 (ETS2) or chr21q22 enhancer inhibitor for use according to any one of embodiments 1 to 49, wherein the ETS2 inhibitor or chr21q22 enhancer inhibitor comprises an agent that targets non-coding RNA at the ETS2 locus or at the chr21q22 enhancer locus.
69. The method or Erythroblast Transformation Specific proto-oncogene 2 (ETS2) for use according to any one of embodiments 1 to 49, wherein the ETS2 inhibitor comprises a CRISPR nuclease system comprising a CRISPR-associated protein (Cas protein) and a guide RNA (gRNA) that specifically binds to an ETS2 genomic nucleic acid sequence, optionally wherein the ETS2 genomic nucleic acid sequence comprises the sequence of SEQ ID NO:5.
70. The method or Erythroblast Transformation Specific proto-oncogene 2 (ETS2) for use according to any one of embodiments 1 to 49 or 69, wherein the ETS2 inhibitor comprises a CRISPR nuclease system comprising a CRISPR-associated protein (Cas protein) and a guide RNA (gRNA) comprising a sequence selected from: SEQ ID NO:8 and SEQ ID NO:9.
71. The method or chr21q22 enhancer inhibitor for use according to any one of embodiments 1 to 49, wherein the chr21q22 enhancer inhibitor comprises a CRISPR nuclease system comprising a CRISPR-associated protein (Cas protein) and a guide RNA (gRNA) that specifically binds to the chr21q22 enhancer nucleic acid sequence, optionally wherein the chr21q22 enhancer nucleic acid sequence comprises the sequence of SEQ ID NO: 1.
72. The method or chr21q22 enhancer inhibitor for use according to any one of embodiments 1 to 49 or 71, wherein the chr21q22 enhancer inhibitor comprises a CRISPR nuclease system comprising a CRISPR-associated protein (Cas protein) and a guide RNA (gRNA) comprising a sequence selected from: SEQ ID NO:6 or SEQ ID NO:7.
73. The method or chr21q22 enhancer inhibitor for use according to any one of embodiments 1 to 49, wherein the chr21q22 enhancer inhibitor comprises a genetic editing technique selected from the list consisting of: gene/base/PRIME editing strategies (for example CRISPR-based genome-targeting tools), gene therapies (for example Adeno-Associated Viruses (AAV), retro- or lenti-viral vectors) and RNA therapies (including, but not limited to, antisense oligonucleotides).
74. The method or Erythroblast Transformation Specific proto-oncogene 2 (ETS2) inhibitor for use according to embodiment 50, wherein the ETS2 inhibitor comprises a small interfering RNA (siRNA) molecule comprising a sense strand.
75. The method or Erythroblast Transformation Specific proto-oncogene 2 (ETS2) inhibitor for use according to embodiment 74, wherein the sense strand consists of 15 to 30 linked nucleosides.
76. The method or Erythroblast Transformation Specific proto-oncogene 2 (ETS2) inhibitor for use according to any one of embodiments 74 to 75, wherein the sense strand comprises a sequence having at least 95% identity to an equal length portion of a pregenomic RNA and/or an mRNA encoding Erythroblast Transformation Specific proto-oncogene 2 (ETS2).
77. The method or Erythroblast Transformation Specific proto-oncogene 2 (ETS2) inhibitor for use according to any one of embodiments 74 to 76, wherein the sense strand comprises a sequence having 100% identity to an equal length portion of a pregenomic RNA and/or an mRNA encoding Erythroblast Transformation Specific proto-oncogene 2 (ETS2).
78. The method or Erythroblast Transformation Specific proto-oncogene 2 (ETS2) inhibitor for use according to any one of embodiments 74 to 77, wherein the small interfering RNA (siRNA) molecule comprises an antisense strand.
79. The method or Erythroblast Transformation Specific proto-oncogene 2 (ETS2) inhibitor for use according to any one of embodiments 74 to 78, wherein the antisense strand is at least 80%, at least 90%, at least 95% or at least 99% complementary to the sense strand.
80. The method or Erythroblast Transformation Specific proto-oncogene 2 (ETS2) inhibitor for use according to any one of embodiments 74 to 79, wherein the antisense strand is fully complementary to the sense strand.
81. The method or Erythroblast Transformation Specific proto-oncogene 2 (ETS2) inhibitor for use according to any preceding embodiment, wherein the ETS2 inhibitor is capable of inhibiting the expression of ETS2 in vitro by at least 50%, at least 60%, at least 70%, at least 80%, at least 90%, at least 95% or at least 99%.
82. The method or chr21q22 enhancer inhibitor for use according to any one of embodiments 1 to 71, wherein the chr21q22 enhancer inhibitor is capable of inhibiting the expression of ETS2 in vitro by at least 50%, at least 60%, at least 70%, at least 80%, at least 90%, at least 95% or at least 99%.
83. The method or Erythroblast Transformation Specific proto-oncogene 2 (ETS2) inhibitor for use according to embodiment 50, wherein the ETS2 inhibitor comprises an antisense nucleic acid molecule.
84. The method or Erythroblast Transformation Specific proto-oncogene 2 (ETS2) inhibitor for use according to embodiment 83, wherein the antisense compound is an antisense oligonucleotide.
85. The method or Erythroblast Transformation Specific proto-oncogene 2 (ETS2) inhibitor for use according to embodiment 83 or 84, wherein the antisense compound specifically binds to an ETS2 mRNA sequence comprising SEQ ID NO:2 or SEQ ID NO:3, or an ETS2 genomic DNA sequence comprising SEQ ID NO:5.
86. The method or Erythroblast Transformation Specific proto-oncogene 2 (ETS2) inhibitor for use according to any one of embodiments 83 to 85, wherein the antisense compound specifically binds to an ETS2 mRNA sequence comprising SEQ ID NO:2.
87. The method or Erythroblast Transformation Specific proto-oncogene 2 (ETS2) inhibitor for use according to any one of embodiments 83 to 86, wherein the antisense oligonucleotide comprises at least one modified internucleoside linkage.
88. The method or Erythroblast Transformation Specific proto-oncogene 2 (ETS2) inhibitor for use according to embodiment 87, wherein the modified internucleoside linkage is a phosphorothioate linkage.
89. The method or Erythroblast Transformation Specific proto-oncogene 2 (ETS2) inhibitor for use according to any one of embodiments 83 to 88, wherein the antisense oligonucleotide comprises at least one modified sugar moiety.
90. The method or Erythroblast Transformation Specific proto-oncogene 2 (ETS2) inhibitor for use according to embodiment 89, wherein the modified sugar moiety is a 2′-O-methoxyethyl sugar moiety.
91. The method or Erythroblast Transformation Specific proto-oncogene 2 (ETS2) inhibitor for use according to any one of embodiments 83 to 90, wherein the antisense oligonucleotide comprises at least one modified nucleobase.
92. The method or Erythroblast Transformation Specific proto-oncogene 2 (ETS2) inhibitor for use according to embodiment 91, wherein the modified nucleobase is a 5-methylcytosine.
93. The method or Erythroblast Transformation Specific proto-oncogene 2 (ETS2) inhibitor for use according to any one of embodiments 83 to 92, wherein the antisense oligonucleotide is a chimeric oligonucleotide.
94. The method or Erythroblast Transformation Specific proto-oncogene 2 (ETS2) inhibitor for use according to any preceding embodiment, wherein the ETS2 inhibitor comprises a compound comprising an ETS2 inhibitor and a conjugate group.
95. The method or chr21q22 enhancer inhibitor for use according to any one of embodiments 1 to 71, 83 or 94, wherein the chr21q22 enhancer inhibitor comprises a compound comprising an chr21q22 enhancer inhibitor and a conjugate group.
96. The method, Erythroblast Transformation Specific proto-oncogene 2 (ETS2) or chr21q22 enhancer inhibitor for use according to any one of embodiments 94 or 95, wherein the conjugate group comprises one or more antibodies or antigen-binding portions thereof, for example a Fab fragment.
97. The method, Erythroblast Transformation Specific proto-oncogene 2 (ETS2) or chr21q22 enhancer inhibitor for use according to any one of embodiments 94 or 95, wherein the conjugate group comprises one or more carbohydrates.
98. The method, Erythroblast Transformation Specific proto-oncogene 2 (ETS2) or chr21q22 enhancer inhibitor for use according to any one of embodiments 94 to 97, wherein the conjugate group comprises one or more galactose moieties, one or more lactose moieties, one or more N-Acetyl-Galactosamine moieties, and/or one or more mannose moieties.
99. The method or Erythroblast Transformation Specific proto-oncogene 2 (ETS2) inhibitor for use according to any one of embodiments 94 to 98, wherein the conjugate group comprises one or more N-Acetyl-Galactosamine moieties.
100. The method, Erythroblast Transformation Specific proto-oncogene 2 (ETS2) or chr21q22 enhancer inhibitor for use according to any one of embodiments 94 or 95, wherein the conjugate group comprises a nanoparticle for delivery of the ETS2 or chr21q22 enhancer inhibitor to a macrophage.
101 The method, Erythroblast Transformation Specific proto-oncogene 2 (ETS2) or chr21q22 enhancer inhibitor for use according to any one of embodiments 94 to 100, wherein the conjugate group comprises a lipid carrier.
102. The method, Erythroblast Transformation Specific proto-oncogene 2 (ETS2) or chr21q22 enhancer inhibitor for use according to embodiment 101, wherein the lipid carrier comprises one or more components selected from the list consisting of: poly(lactic acid) (PLA) microspheres, poly(D,L-lactic-coglycolic-acid) (PLGA) microspheres, liposomes, lipid nanoparticles, micelles, inverse micelles, lipid cochleates, and lipid microtubules.
103. A method of providing a diagnosis or prognosis of an inflammatory or autoimmune disease in a subject based on the expression status of Erythroblast Transformation Specific proto-oncogene 2 (ETS2).
104. A method of providing a diagnosis or prognosis of an inflammatory or autoimmune disease in a subject based on the expression status of the chr21q22 enhancer (SEQ ID NO: 1).
105. The method of providing a diagnosis or prognosis according to any one of embodiments 103 or 104, wherein determining the expression status of ETS2 or the chr21q22 enhancer comprises determination of one or more statuses in a biological sample obtained from the subject selected from the list consisting of: ETS2 mRNA levels, ETS2 protein levels, ETS2 DNA methylation status, ETS2 epigenetic status (such as histone marking, RNA changes or conformation changes), chr21q22 enhancer DNA methylation status and chr21q22 enhancer epigenetic status (such as histone marking, RNA changes or conformation changes).
106. The method of providing a diagnosis or prognosis according to any one of embodiments 103 to 105, wherein determining the expression status of ETS2 comprises determination of ETS2 mRNA levels or ETS2 protein levels.
107. The method of providing a diagnosis or prognosis according to any one of embodiments 103 to 106, wherein determining the expression status of ETS2 comprises the step of quantifying the expression status of an RNA transcript or cDNA molecule and wherein the expression status of the RNA or cDNA is quantified using any one or more of the following techniques: microarray analysis, real-time quantitative PCR, DNA sequencing, RNA sequencing, Northern blot analysis, in situ hybridisation and/or detection and quantification of a specific binding molecule (e.g. an antibody).
108. The method of providing a diagnosis or prognosis according to any one of embodiments 103 to 107, further comprising the step of comparing or normalising the expression status of ETS2 with the expression status of a reference gene.
109. A method of treating or preventing a disease in a subject, the method comprising administering an inhibitor of a protein that activates Erythroblast Transformation Specific proto-oncogene 2 (ETS2) to the subject.
110. The method of treating or preventing a disease in a subject according to embodiment 109, wherein the protein that activates Erythroblast Transformation Specific proto-oncogene 2 (ETS2) is selected from the list consisting of: MEK, HSP90, RAF, SRC and ERK.
111. The method of treating or preventing a disease in a subject according to embodiment 110, wherein the MEK inhibitor is selected from the list consisting of Selumetinib, Trametinib and Cobimetinib, preferably wherein the MEK inhibitor is Selumetinib.
112. The method of treating a disease in a subject according to any one of embodiments 109 to 111, wherein the inhibitor of a protein that activates Erythroblast Transformation Specific proto-oncogene 2 (ETS2) is an antibody-drug conjugate, an antibody-RNA conjugate, an esterase-sensitive motif-conjugated drug or a nanobody-drug conjugate.
113. The method of treating a disease in a subject according to embodiment 112, wherein the antibody-drug conjugate comprises an anti-CD163 antigen binding molecule, optionally wherein the antigen binding molecule is an antibody.
114. The method of treating a disease in a subject according to any one of embodiments 112 or 113, wherein the antibody-drug conjugate comprises a cleavable linker, optionally wherein the cleavable linker is glucuronide-based (e.g. GlyPro).
115. The method of treating a disease in a subject according to any one of embodiments 112 or 113, wherein the antibody-drug conjugate comprises a non-cleavable linker, optionally wherein the non-cleavable linker is maleimide-PEG3-based.
116. The method of treating a disease in a subject according to embodiment 112, wherein the esterase-sensitive motif-conjugated drug comprises cyclopentyl L-leucinate or cyclopentyl(S)-2-amino-2-cyclohexylacetate.
117 The method of treating a disease in a subject according to embodiment 112 or 116, wherein the esterase-sensitive motif-conjugated drug comprises a linker.
118. The method of treating a disease in a subject according to embodiment 117, wherein the linker is one or two carbons in length.
119. The method of treating a disease in a subject according to any one of embodiments 109 to 118, wherein the subject has an inflammatory disease and/or autoimmune disease.
120. The method of treating a disease in a subject according to embodiment 119, wherein the inflammatory disease is selected from the list consisting of: Crohn's disease, ulcerative colitis, inflammatory bowel disease (Crohn's disease and/or ulcerative colitis), ankylosing spondylitis, primary sclerosing cholangitis, Takayasu arteritis and rheumatoid arthritis.
121. An inhibitor of a protein that activates Erythroblast Transformation Specific proto-oncogene 2 (ETS2) for use in a method of treating or preventing a disease in a subject, the method comprising administering an inhibitor of a protein that activates Erythroblast Transformation Specific proto-oncogene 2 (ETS2) to the subject.
122. The inhibitor of a protein that activates Erythroblast Transformation Specific proto-oncogene 2 (ETS2) for use according to embodiment 121, wherein the protein that activates Erythroblast Transformation Specific proto-oncogene 2 (ETS2) is selected from the list consisting of: MEK, HSP90, RAF, SRC and ERK.
123. The inhibitor of a protein that activates Erythroblast Transformation Specific proto-oncogene 2 (ETS2) for use according to embodiment 122, wherein the MEK inhibitor is selected from the list consisting of Selumetinib, Trametinib and Cobimetinib, preferably wherein the MEK inhibitor is Selumetinib.
124. The inhibitor of a protein that activates Erythroblast Transformation Specific proto-oncogene 2 (ETS2) for use according to any one of embodiments 121 to 123, wherein the inhibitor of a protein that activates Erythroblast Transformation Specific proto-oncogene 2 (ETS2) is an antibody-drug conjugate, an antibody-RNA conjugate, an esterase-sensitive motif-conjugated drug or a nanobody-drug conjugate.
125. The inhibitor of a protein that activates Erythroblast Transformation Specific proto-oncogene 2 (ETS2) for use according to embodiment 124, wherein the antibody-drug conjugate comprises an anti-CD163 antigen binding molecule, optionally wherein the antigen binding molecule is an antibody.
126. The inhibitor of a protein that activates Erythroblast Transformation Specific proto-oncogene 2 (ETS2) for use according to any one of embodiments 124 or 125, wherein the antibody-drug conjugate comprises a cleavable linker, optionally wherein the cleavable linker is glucuronide-based (e.g. GlyPro).
127. The inhibitor of a protein that activates Erythroblast Transformation Specific proto-oncogene 2 (ETS2) for use according to any one of embodiments 124 or 125, wherein the antibody-drug conjugate comprises a non-cleavable linker, optionally wherein the non-cleavable linker is maleimide-PEG3-based.
128. The inhibitor of a protein that activates Erythroblast Transformation Specific proto-oncogene 2 (ETS2) for use according to embodiment 124, wherein the esterase-sensitive motif-conjugated drug comprises cyclopentyl L-leucinate or cyclopentyl(S)-2-amino-2-cyclohexylacetate.
129. The inhibitor of a protein that activates Erythroblast Transformation Specific proto-oncogene 2 (ETS2) for use according to embodiment 124 or 128, wherein the esterase-sensitive motif-conjugated drug comprises a linker.
130. The inhibitor of a protein that activates Erythroblast Transformation Specific proto-oncogene 2 (ETS2) for use according to embodiment 129, wherein the linker is one or two carbons in length.
131 The inhibitor of a protein that activates Erythroblast Transformation Specific proto-oncogene 2 (ETS2) for use according to any one of embodiments 121 to 130, wherein the subject has an inflammatory disease and/or autoimmune disease.
132. The inhibitor of a protein that activates Erythroblast Transformation Specific proto-oncogene 2 (ETS2) for use according to embodiment 131, wherein the inflammatory disease is selected from the list consisting of: Crohn's disease, ulcerative colitis, inflammatory bowel disease (Crohn's disease and/or ulcerative colitis), ankylosing spondylitis, primary sclerosing cholangitis, Takayasu arteritis and rheumatoid arthritis.
133. A method of reducing macrophage activation by contacting said macrophage with an inhibitor of a protein that activates Erythroblast Transformation Specific proto-oncogene 2 (ETS2).
134. A method of reducing proinflammatory cytokine production by contacting a macrophage with an inhibitor of a protein that activates Erythroblast Transformation Specific proto-oncogene 2 (ETS2).
135. A method of reducing reactive oxygen species (ROS) production by contacting a macrophage with an inhibitor of a protein that activates Erythroblast Transformation Specific proto-oncogene 2 (ETS2).
136. A method of reducing macrophage phagocytosis by contacting said macrophage with an inhibitor of a protein that activates Erythroblast Transformation Specific proto-oncogene 2 (ETS2).
137. A method of reducing macrophage migration by contacting said macrophage with an inhibitor of a protein that activates Erythroblast Transformation Specific proto-oncogene 2 (ETS2).
138. An inhibitor of a protein that activates Erythroblast Transformation Specific proto-oncogene 2 (ETS2) for use in a method of reducing macrophage activation by contacting said macrophage with an inhibitor of a protein that activates Erythroblast Transformation Specific proto-oncogene 2 (ETS2).
139. An inhibitor of a protein that activates Erythroblast Transformation Specific proto-oncogene 2 (ETS2) for use in a method of reducing proinflammatory cytokine production by contacting a macrophage with an inhibitor of a protein that activates Erythroblast Transformation Specific proto-oncogene 2 (ETS2).
140. An inhibitor of a protein that activates Erythroblast Transformation Specific proto-oncogene 2 (ETS2) for use in a method of reducing reactive oxygen species (ROS) production by contacting a macrophage with an inhibitor of a protein that activates Erythroblast Transformation Specific proto-oncogene 2 (ETS2).
141. An inhibitor of a protein that activates Erythroblast Transformation Specific proto-oncogene 2 (ETS2) for use in a method of reducing macrophage phagocytosis by contacting said macrophage with an inhibitor of a protein that activates Erythroblast Transformation Specific proto-oncogene 2 (ETS2).
142. An inhibitor of a protein that activates Erythroblast Transformation Specific proto-oncogene 2 (ETS2) for use in a method of reducing macrophage migration by contacting said macrophage with an inhibitor of a protein that activates Erythroblast Transformation Specific proto-oncogene 2 (ETS2).
143. The method or inhibitor of a protein that activates Erythroblast Transformation Specific proto-oncogene 2 (ETS2) for use according to any one of embodiments 133 to 142, wherein the protein that activates Erythroblast Transformation Specific proto-oncogene 2 (ETS2) is selected from the list consisting of: MEK, HSP90, RAF, SRC and ERK.
144 The method or inhibitor of a protein that activates Erythroblast Transformation Specific proto-oncogene 2 (ETS2) for use according to embodiment 143, wherein the MEK inhibitor is selected from the list consisting of Selumetinib, Trametinib and Cobimetinib, preferably wherein the MEK inhibitor is Selumetinib.
145. A method of screening for agents that reduce macrophage activation, the method comprising:
   a. contacting a macrophage with a candidate agent; and
   b. determining the expression status of ETS2;
   wherein agents that reduce expression of ETS2 are identified as agents that reduce macrophage activation.
146. The method of screening for agents according to embodiment 145, wherein determining the expression status of ETS2 comprises determination of ETS2 mRNA levels or ETS2 protein levels.
147. A method of screening for candidate genes involved in macrophage activation, the method comprising:
   a. introducing to a macrophage a CRISPR nuclease system comprising a CRISPR-associated protein (Cas protein) and a guide RNA (gRNA) that specifically binds to an ETS2 genomic nucleic acid sequence;
   b. measuring the expression status of one or more candidate genes; and
   c. identifying genes as being associated with macrophage activation by comparing the expression status to a reference expression status of the same genes from a control cell without the CRISPR nuclease system, wherein genes that have increased or decreased expression relative to the reference expression status are associated with macrophage activation.
148. The method of screening for candidate genes involved in macrophage activation according to embodiment 147, wherein the ETS2 genomic nucleic acid sequence comprises the sequence of SEQ ID NO:5.
149. A method of screening for candidate genes involved in macrophage activation, the method comprising:
   a. introducing to a macrophage a CRISPR nuclease system comprising a CRISPR-associated protein (Cas protein) and a guide RNA (gRNA) that specifically binds to a chr21q22 enhancer nucleic acid sequence;
   b. measuring the expression status of one or more candidate genes; and
   c. identifying genes as being associated with macrophage activation by comparing the expression status to a reference expression status of the same genes from a control cell without the CRISPR nuclease system, wherein genes that have increased or decreased expression relative to the reference expression status are associated with macrophage activation.
150. The method of screening for candidate genes involved in macrophage activation according to embodiment 148, wherein the chr21q22 enhancer nucleic acid sequence comprises the sequence of SEQ ID NO: 1.
151. A method of screening for candidate genes involved in macrophage activation, the method comprising:
   a. introducing to a macrophage a CRISPR nuclease system comprising a CRISPR-associated protein (Cas protein) and a guide RNA (gRNA) that specifically binds to the genomic nucleic acid sequence a candidate gene;
   b. measuring the expression status of ETS2; and
   c. identifying genes as being associated with macrophage activation by comparing the expression status of ETS2 to a reference expression status of ETS2 from a control cell without the CRISPR nuclease system, wherein candidate genes that modulate ETS2 expression status are associated with macrophage activation.
152. The method of screening for candidate genes involved in macrophage activation according to any one of embodiments 147 to 151, wherein the expression status of the one or more candidate genes is quantified using any one or more of the following techniques: microarray analysis, real-time quantitative PCR, DNA sequencing, RNA sequencing, Northern blot analysis, in situ hybridisation and/or detection and quantification of a specific binding molecule (e.g. an antibody).
153. An inhibitor of a protein that activates Erythroblast Transformation Specific proto-oncogene 2 (ETS2) for use in a method of treating or preventing a disease in a subject, the method comprising administering an inhibitor of a protein that activates Erythroblast Transformation Specific proto-oncogene 2 (ETS2) to the subject, wherein the protein that activates Erythroblast Transformation Specific proto-oncogene 2 (ETS2) is selected from the list consisting of: MEK, HSP90, RAF, SRC and ERK, and wherein the disease is an inflammatory and/or autoimmune disease.
154. An inhibitor of a protein that activates Erythroblast Transformation Specific proto-oncogene 2 (ETS2) for use in a method of treating or preventing a disease in a subject, the method comprising administering an inhibitor of a protein that activates Erythroblast Transformation Specific proto-oncogene 2 (ETS2) to the subject, wherein the protein that activates Erythroblast Transformation Specific proto-oncogene 2 (ETS2) is selected from the list consisting of: MEK, HSP90, RAF, SRC and ERK, and wherein the disease is an autoinflammatory disease.
155. An inhibitor of a protein that activates Erythroblast Transformation Specific proto-oncogene 2 (ETS2) for use in a method of treating or preventing a disease in a subject, the method comprising administering an inhibitor of a protein that activates Erythroblast Transformation Specific proto-oncogene 2 (ETS2) to the subject, wherein the protein that activates Erythroblast Transformation Specific proto-oncogene 2 (ETS2) is selected from the list consisting of: MEK, HSP90, RAF, SRC and ERK, and wherein the disease is a chr21q22-associated disease.
156. The inhibitor of a protein that activates Erythroblast Transformation Specific proto-oncogene 2 (ETS2) for use according to clause 153, wherein the disease is an autoinflammatory disease.
157. The inhibitor of a protein that activates Erythroblast Transformation Specific proto-oncogene 2 (ETS2) for use according to clause 153, wherein the disease is a chr21q22-associated disease.
158 The inhibitor of a protein that activates Erythroblast Transformation Specific proto-oncogene 2 (ETS2) for use according to any of clauses 153-157, wherein the disease is selected from the list consisting of Crohn's disease, ulcerative colitis, primary sclerosing cholangitis, ankylosing spondylitis, and Takayasu arteritis.

### REFERENCES

[1] Dowden, H. & Munro, J. Trends in clinical success rates and therapeutic focus. Nat Rev Drug Discov 18, 495-496, doi: 10.1038/d41573-019-00074-z (2019).
[2] Harrison, R. K. Phase II and phase III failures: 2013-2015. Nat Rev Drug Discov 15, 817-818, doi: 10.1038/nrd.2016.184 (2016).
[3] Claussnitzer, M. et al. A brief history of human disease genetics. Nature 577, 179-189, doi: 10.1038/s41586-019-1879-7 (2020).
[4] Nelson, M. R. et al. The support of human genetic evidence for approved drug indications. Nat Genet 47, 856-860, doi: ng.3314 [pii] 10.1038/ng.3314 (2015).
[5] King, E. A., Davis, J. W. & Degner, J. F. Are drug targets with genetic support twice as likely to be approved? Revised estimates of the impact of genetic support for drug mechanisms on the probability of drug approval. PLOS Genet 15, e1008489, doi: 10.1371/journal.pgen. 1008489 (2019).
[6] Hnisz, D. et al. Super-enhancers in the control of cell identity and disease. Cell 155, 934-947, doi: S0092-8674 (13) 01227-0 [pii] 10.1016/j.cell.2013.09.053 (2013).
[7] de Lange, K. M. et al. Genome-wide association study implicates immune activation of multiple integrin genes in inflammatory bowel disease. Nat Genet 49, 256-261, doi: 10.1038/ng.3760 (2017).
[8] International Genetics of Ankylosing Spondylitis, C. et al. Identification of multiple risk variants for ankylosing spondylitis through high-density genotyping of immune-related loci. Nat Genet 45, 730-738, doi: 10.1038/ng.2667 (2013).
[9] Liu, J. Z. et al. Dense genotyping of immune-related disease regions identifies nine new risk loci for primary sclerosing cholangitis. Nat Genet 45, 670-675, doi: 10.1038/ng.2616 (2013).
[10] Ortiz-Fernandez, L. et al. Identification of susceptibility loci for Takayasu arteritis through a large multi-ancestral genome-wide association study. Am J Hum Genet 108, 84-99, doi: 10.1016/j.ajhg.2020.11.014 (2021).
[11] Kugathasan, S. et al. Loci on 20q13 and 21q22 are associated with pediatric-onset inflammatory bowel disease. Nat Genet 40, 1211-1215, doi: 10.1038/ng.203 (2008).
[12] Park, M. D., Silvin, A., Ginhoux, F. & Merad, M. Macrophages in health and disease. Cell 185, 4259-4279, doi: 10.1016/j.cell.2022.10.007 (2022).
[13] Javierre, B. M. et al. Lineage-Specific Genome Architecture Links Enhancers and Non-coding Disease Variants to Target Gene Promoters. Cell 167, 1369-1384 e1319, doi: 10.1016/j.cell.2016.09.037 (2016).
[14] Fairfax, B. P. et al. Innate immune activity conditions the effect of regulatory variants upon monocyte gene expression. Science 343, 1246949, doi: 343/6175/1246949 [pii] 10.1126/science. 1246949 (2014).
[15] Xue, J. et al. Transcriptome-based network analysis reveals a spectrum model of human macrophage activation. Immunity 40, 274-288, doi: 10.1016/j.immuni.2014.01.006 (2014).
[16] Kuo, D. et al. HBEGF (+) macrophages in rheumatoid arthritis induce fibroblast invasiveness. Sci Transl Med 11, doi: 10.1126/scitranslmed.aau8587 (2019).
[17] Melnikov, A. et al. Systematic dissection and optimization of inducible enhancers in human cells using a massively parallel reporter assay. Nat Biotechnol 30, 271-277, doi: 10.1038/nbt.2137 (2012).
[18] Bourges, C. et al. Resolving mechanisms of immune-mediated disease in primary CD4 T cells. EMBO Mol Med 12, e12112, doi: 10.15252/emmm.202012112 (2020).
[19] Nerlov, C. & Graf, T. PU. 1 induces myeloid lineage commitment in multipotent hematopoietic progenitors. Genes Dev 12, 2403-2412, doi: 10.1101/gad. 12.15.2403 (1998).
[20] Ghisletti, S. et al. Identification and characterization of enhancers controlling the inflammatory gene expression program in macrophages. Immunity 32, 317-328, doi: 10.1016/j.immuni.2010.02.008 (2010).
[21] Heinz, S. et al. Simple combinations of lineage-determining transcription factors prime cis-regulatory elements required for macrophage and B cell identities. Mol Cell 38, 576-589, doi: 10.1016/j.molcel.2010.05.004 (2010).
[22] de Santiago, I. et al. BaaIChIP: Bayesian analysis of allele-specific transcription factor binding in cancer genomes. Genome Biol 18, 39, doi: 10.1186/s13059-017-1165-7 (2017).
[23] Martinez, L. A. Mutant p53 and ETS2, a Tale of Reciprocity. Front Oncol 6, 35, doi: 10.3389/fonc.2016.00035 (2016).
[24] Wei, G. et al. Activated Ets2 is required for persistent inflammatory responses in the motheaten viable model. J Immunol 173, 1374-1379, doi: 10.4049/jimmunol. 173.2. 1374 (2004).
[25] Zhao, J., Huang, K., Peng, H. Z. & Feng, J. F. Protein C-ets-2 epigenetically suppresses TLRs-induced interleukin 6 production in macrophages. Biochem Biophys Res Commun 522, 960-964, doi: 10.1016/j.bbrc.2019.11.123 (2020).
[26] Chung, S. W., Chen, Y. H. & Perrella, M. A. Role of Ets-2 in the regulation of heme oxygenase-1 by endotoxin. J Biol Chem 280, 4578-4584, doi: 10.1074/jbc. M409125200 (2005).
[27] Quinn, S. R. et al. The role of Ets2 transcription factor in the induction of microRNA-155 (miR-155) by lipopolysaccharide and its targeting by interleukin-10. J Biol Chem 289, 4316-4325, doi: 10.1074/jbc.M113.522730 (2014).
[28] Ma, X. et al. Ets2 suppresses inflammatory cytokines through MAPK/NF-kappaB signaling and directly binds to the IL-6 promoter in macrophages. Aging (Albany NY) 11, 10610-10625, doi: 10.18632/aging. 102480 (2019).
[29] Aperlo, C., Pognonec, P., Stanley, E. R. & Boulukos, K. E. Constitutive c-ets2 expression in M1D+ myeloblast leukemic cells induces their differentiation to macrophages. Mol Cell Biol 16, 6851-6858, doi: 10.1128/MCB. 16.12.6851 (1996).
[30] Henkel, G. W. et al. PU.1 but not ets-2 is essential for macrophage development from embryonic stem cells. Blood 88, 2917-2926 (1996).
[31] Mittal, M., Siddiqui, M. R., Tran, K., Reddy, S. P. & Malik, A. B. Reactive oxygen species in inflammation and tissue injury. Antioxid Redox Signal 20, 1126-1167, doi: 10.1089/ars.2012.5149 (2014).
[32] Kelly, B. & O'Neill, L. A. Metabolic reprogramming in macrophages and dendritic cells in innate immunity. Cell Res 25, 771-784, doi: 10.1038/cr.2015.68 (2015).
[33] Martin, J. C. et al. Single-Cell Analysis of Crohn's Disease Lesions Identifies a Pathogenic Cellular Module Associated with Resistance to Anti-TNF Therapy. Cell 178, 1493-1508 e1420, doi: 10.1016/j.cell.2019.08.008 (2019).
[34] Kaser, A. & Blumberg, R. S. Autophagy, microbial sensing, endoplasmic reticulum stress, and epithelial function in inflammatory bowel disease. Gastroenterology 140, 1738-1747, doi: S0016-5085 (11) 00202-2 [pii] 10.1053/j.gastro.2011.02.048 (2011).
[35] Sazonovs, A. et al. Large-scale sequencing identifies multiple genes and rare variants associated with Crohn's disease susceptibility. Nat Genet 54, 1275-1283, doi: 10.1038/s41588-022-01156-2 (2022).
[36] Slowikowski, K., Hu, X. & Raychaudhuri, S. SNPsea: an algorithm to identify cell types, tissues and pathways affected by risk loci. Bioinformatics 30, 2496-2497, doi: 10.1093/bioinformatics/btu326 (2014).
[37] Dunham, I. et al. An integrated encyclopedia of DNA elements in the human genome. Nature 489, 57-74, doi: nature11247 [pii] 10.1038/nature11247 (2012).
[38] Cramer, T. et al. HIF-1alpha is essential for myeloid cell-mediated inflammation. Cell 112, 645-657, doi: 10.1016/s0092-8674 (03) 00154-5 (2003).
[39] Tannahill, G. M. et al. Succinate is an inflammatory signal that induces IL-1beta through HIF-1alpha. Nature 496, 238-242, doi: 10.1038/nature11986 (2013).
[40] Shiratori, R. et al. Glycolytic suppression dramatically changes the intracellular metabolic profile of multiple cancer cell lines in a mitochondrial metabolism-dependent manner. Sci Rep 9, 18699, doi: 10.1038/s41598-019-55296-3 (2019).
[41] Skolik, R. A., Solocinski, J., Konkle, M. E., Chakraborty, N. & Menze, M. A. Global changes to HepG2 cell metabolism in response to galactose treatment. Am J Physiol Cell Physiol 320, C778-C793, doi: 10.1152/ajpcell.00460.2020 (2021).
[42] Landt, S. G. et al. ChIP-seq guidelines and practices of the ENCODE and modENCODE consortia. Genome Res 22, 1813-1831, doi: 10.1101/gr. 136184.111 (2012).
[43] Sevilla, L. et al. Bcl-XL expression correlates with primary macrophage differentiation, activation of functional competence, and survival and results from synergistic transcriptional activation by Ets2 and PU.1. J Biol Chem 276, 17800-17807, doi: 10.1074/jbc. M008270200 (2001)
[44] Newman, J. A., Cooper, C. D., Aitkenhead, H. & Gileadi, O. Structural insights into the autoregulation and cooperativity of the human transcription factor Ets-2. J Biol Chem 290, 8539-8549, doi: 10.1074/jbc. M114.619270 (2015).
[45] Stathias, V. et al. LINCS Data Portal 2.0: next generation access point for perturbation-response signatures. Nucleic Acids Res 48, D431-D439, doi: 10.1093/nar/gkz1023 (2020).
[46] Koboziev, I., Karlsson, F., Zhang, S. & Grisham, M. B. Pharmacological intervention studies using mouse models of the inflammatory bowel diseases: translating preclinical data into new drug therapies. Inflamm Bowel Dis 17, 1229-1245, doi: 10.1002/ibd.21557 (2011).
[47] Vadstrup, K. et al. Validation and Optimization of an Ex Vivo Assay of Intestinal Mucosal Biopsies in Crohn's Disease: Reflects Inflammation and Drug Effects. PLOS One 11, e0155335, doi: 10.1371/journal.pone.0155335 (2016).
[48] Argmann, C. et al. Biopsy and blood-based molecular biomarker of inflammation in IBD. Gut, doi: 10.1136/gutjnl-2021-326451 (2022).
[49] Zou, Y., Carbonetto, P., Wang, G. & Stephens, M. Fine-mapping from summary data with the “Sum of Single Effects” model. PLOS Genet 18, e1010299, doi: 10.1371/journal.pgen. 1010299 (2022).
[50] Wallace, C. A more accurate method for colocalisation analysis allowing for multiple causal variants. PLoS Genet 17, e1009440, doi: 10.1371/journal. pgen. 1009440 (2021).
[51] Bourges, C. et al. Resolving mechanisms of immune-mediated disease in primary CD4 T cells. EMBO Mol Med 12, e12112, doi: 10.15252/emmm.202012112 (2020).
[52] Hanzelmann, S., Castelo, R. & Guinney, J. GSVA: gene set variation analysis for microarray and RNA-seq data. BMC Bioinformatics 14, 7, doi: 10.1186/1471-2105-14-7 (2013).
[53] Peters, J. E. et al. Insight into Genotype-Phenotype Associations through eQTL Mapping in Multiple Cell Types in Health and Immune-Mediated Disease. PLOS Genet 12, e1005908, doi: 10.1371/journal. pgen. 1005908 (2016).
[54] Conant, D. et al. Inference of CRISPR Edits from Sanger Trace Data. CRISPR J 5, 123-130, doi: 10.1089/crispr.2021.0113 (2022).
[55] Kalita, C. A. et al. QuASAR-MPRA: accurate allele-specific analysis for massively parallel reporter assays. Bioinformatics 34, 787-794, doi: 10.1093/bioinformatics/btx598 (2018).
[56] Randzavola, L. O. et al. EROS is a selective chaperone regulating the phagocyte NADPH oxidase and purinergic signalling. Elife 11, doi: 10.7554/eLife.76387 (2022).
[57] Kim, D., Paggi, J. M., Park, C., Bennett, C. & Salzberg, S. L. Graph-based genome alignment and genotyping with HISAT2 and HISAT-genotype. Nat Biotechnol 37, 907-915, doi: 10.1038/s41587-019-0201-4 (2019).
[58] Li, H. et al. The Sequence Alignment/Map format and SAMtools. Bioinformatics 25, 2078-2079, doi: 10.1093/bioinformatics/btp352 (2009).
[59] Liao, Y., Smyth, G. K. & Shi, W. featureCounts: an efficient general purpose program for assigning sequence reads to genomic features. Bioinformatics 30, 923-930, doi: 10.1093/bioinformatics/btt656 (2014).
[60] Ritchie, M. E. et al. limma powers differential expression analyses for RNA-sequencing and microarray studies. Nucleic Acids Res 43, e47, doi: 10.1093/nar/gkv007 (2015).
[61] Korotkevich, G. et al. Fast gene set enrichment analysis. bioRxiv, 060012, doi: 10.1101/060012 (2021).
[62] Bussi, C. et al. Lysosomal damage drives mitochondrial proteome remodelling and reprograms macrophage immunometabolism. Nat Commun 13, 7338, doi: 10.1038/s41467-022-34632-8 (2022).
[63] Behrends, V., Tredwell, G. D. & Bundy, J. G. A software complement to AMDIS for processing GC-MS metabolomic data. Anal Biochem 415, 206-208, doi: 10.1016/j.ab.2011.04.009 (2011).
[64] Meers, M. P., Bryson, T. D., Henikoff, J. G. & Henikoff, S. Improved CUT&RUN chromatin profiling tools. Elife 8, doi: 10.7554/eLife.46314 (2019).
[65] Leporcq, C. et al. TFmotifView: a webserver for the visualization of transcription factor motifs in genomic regions. Nucleic Acids Res 48, W208-W217, doi: 10.1093/nar/gkaa252 (2020).
[66] Corces, M. R. et al. An improved ATAC-seq protocol reduces background and enables interrogation of frozen tissues. Nat Methods 14, 959-962, doi: 10.1038/nmeth.4396 (2017).
[67] Calderon, D. et al. Landscape of stimulation-responsive chromatin across diverse human immune cells. Nat Genet 51, 1494-1505, doi: 10.1038/s41588-019-0505-9 (2019).

1. An inhibitor of a protein that activates Erythroblast Transformation Specific proto-oncogene 2 (ETS2) for use in a method of treating or preventing a disease in a subject, the method comprising administering an inhibitor of a protein that activates Erythroblast Transformation Specific proto-oncogene 2 (ETS2) to the subject, wherein the protein that activates Erythroblast Transformation Specific proto-oncogene 2 (ETS2) is selected from the list consisting of: MEK, HSP90, RAF, SRC and ERK, and wherein the disease is an inflammatory and/or autoimmune disease.

2. The inhibitor of a protein that activates Erythroblast Transformation Specific proto-oncogene 2 (ETS2) for use according to claim 1, wherein the MEK inhibitor is selected from the list consisting of Selumetinib, Trametinib and Cobimetinib.

3. The inhibitor of a protein that activates Erythroblast Transformation Specific proto-oncogene 2 (ETS2) for use according to claim 1 or 2, wherein the inhibitor of a protein that activates Erythroblast Transformation Specific proto-oncogene 2 (ETS2) is an antibody-drug conjugate, an antibody-RNA conjugate, an esterase-sensitive motif-conjugated drug or a nanobody-drug conjugate.

4. The inhibitor of a protein that activates Erythroblast Transformation Specific proto-oncogene 2 (ETS2) for use according to claim 3, wherein the antibody-drug conjugate comprises an anti-CD163 antigen binding molecule, optionally wherein the antigen binding molecule is an antibody.

5. The inhibitor of a protein that activates Erythroblast Transformation Specific proto-oncogene 2 (ETS2) for use according to any one of claim 3 or 4, wherein the antibody-drug conjugate comprises a cleavable linker, optionally wherein the cleavable linker is glucuronide-based (e.g. GlyPro).

6. The inhibitor of a protein that activates Erythroblast Transformation Specific proto-oncogene 2 (ETS2) for use according to any one of claim 3 or 4, wherein the antibody-drug conjugate comprises a non-cleavable linker, optionally wherein the non-cleavable linker is maleimide-PEG3-based.

7. The inhibitor of a protein that activates Erythroblast Transformation Specific proto-oncogene 2 (ETS2) for use according to claim 3, wherein the esterase-sensitive motif-conjugated drug comprises cyclopentyl L-leucinate or cyclopentyl(S)-2-amino-2-cyclohexylacetate.

8. The inhibitor of a protein that activates Erythroblast Transformation Specific proto-oncogene 2 (ETS2) for use according to claim 3 or 7, wherein the esterase-sensitive motif-conjugated drug comprises a linker.

9. The inhibitor of a protein that activates Erythroblast Transformation Specific proto-oncogene 2 (ETS2) for use according to claim 8, wherein the linker is one or two carbons in length.

10. The inhibitor of a protein that activates Erythroblast Transformation Specific proto-oncogene 2 (ETS2) for use according to claim 1, wherein the disease is an autoinflammatory disease.

11. The inhibitor of a protein that activates Erythroblast Transformation Specific proto-oncogene 2 (ETS2) for use according to claim 1, wherein the disease is a chr21q22-associated disease.

12. The inhibitor of a protein that activates Erythroblast Transformation Specific proto-oncogene 2 (ETS2) for use according to claim 1, wherein the inflammatory disease is selected from the list consisting of: Crohn's disease, ulcerative colitis, inflammatory bowel disease (Crohn's disease and/or ulcerative colitis), ankylosing spondylitis, primary sclerosing cholangitis, Takayasu arteritis and rheumatoid arthritis.

13. The inhibitor of a protein that activates Erythroblast Transformation Specific proto-oncogene 2 (ETS2) for use according to claim 1, wherein the disease is selected from the list consisting of Crohn's disease, ulcerative colitis, primary sclerosing cholangitis, ankylosing spondylitis, and Takayasu arteritis.

14. A method of screening for agents that reduce macrophage activation, the method comprising:
(a) contacting a macrophage with a candidate agent; and
(b) determining the expression status of ETS2;
wherein agents that reduce expression of ETS2 are identified as agents that reduce macrophage activation.

15. The method of screening for agents according to claim 15, wherein determining the expression status of ETS2 comprises determination of ETS2 mRNA levels or ETS2 protein levels.

16. A method of screening for candidate genes involved in macrophage activation, the method comprising:
(a) introducing to a macrophage a CRISPR nuclease system comprising a CRISPR-associated protein (Cas protein) and a guide RNA (gRNA) that specifically binds to the genomic nucleic acid sequence a candidate gene;
(b) measuring the expression status of ETS2; and
(c) identifying genes as being associated with macrophage activation by comparing the expression status of ETS2 to a reference expression status of ETS2 from a control cell without the CRISPR nuclease system, wherein candidate genes that modulate ETS2 expression status are associated with macrophage activation.

17. The method of screening for candidate genes involved in macrophage activation according to claim 16, wherein the expression status of the one or more candidate genes is quantified using any one or more of the following techniques: microarray analysis, real-time quantitative PCR, DNA sequencing, RNA sequencing, Northern blot analysis, in situ hybridisation and/or detection and quantification of a specific binding molecule (e.g. an antibody).


SEQUENCE LISTING (22 sequences)

SEQ ID NO: 1 - DNA, 442 bp, Homo sapiens
  source 1..442: mol_type = genomic DNA
caggccgttggcgagtgggccctccaaccgtgggccacagctcgtgaatctgtttgttggggagtcatgttcaaacccaaagaacaaggcaaaccctacccccaaggaacccagataactccttatttcccctttttattcttaaaaagtacttctcccaggttccggcggagtgctgttctcattttagaaggctggctaactgtgggtgcaagtcatttctcaaagcagtgtctgttcttttctttaatggaattgttttgttcatttgtttcacttcctctttggggttcccaaggaacatgagttccttttttcctggtattagaagtgcgtcagttcaagtatgagtacatgaaaagtgaagaataagaggctggctccattaggtgatgacatgtacctgctatgagatttgggaagccatggcctgtcattgttg

SEQ ID NO: 2 - RNA, 3668 nt, Homo sapiens
  source 1..3668: mol_type = mRNA
ggttacttcctccagagactgacgagtgcggtgtcgctccagctcagagctcccggagccgcccggccagcgtccggcctccctgatcgtctctggccggcgccctcgccctcgcccggcgcgcaccgagcagccgcgggcgccgagcagccaccgtcccgaccaagcgccggccctgcccgcagcggcaggatgaatgatttcggaatcaagaatatggaccaggtagcccctgtggctaacagttacagagggacactcaagcgccagccagcctttgacacctttgatgggtccctgtttgctgtttttccttctctaaatgaagagcaaacactgcaagaagtgccaacaggcttggattccatttctcatgactccgccaactgtgaattgcctttgttaaccccgtgcagcaaggctgtgatgagtcaagccttaaaagctaccttcagtggcttcaaaaaggaacagcggcgcctgggcattccaaagaacccctggctgtggagtgagcaacaggtatgccagtggcttctctgggccaccaatgagttcagtctggtgaacgtgaatctgcagaggttcggcatgaatggccagatgctgtgtaaccttggcaaggaacgctttctggagctggcacctgactttgtgggtgacattctctgggaacatctggagcaaatgatcaaagaaaaccaagaaaagacagaagatcaatatgaagaaaattcacacctcacctccgttcctcattggattaacagcaatacattaggttttggcacagagcaggcgccctatggaatgcagacacagaattaccccaaaggcggcctcctggacagcatgtgtccggcctccacacccagcgtactcagctctgagcaggagtttcagatgttccccaagtctcggctcagctccgtcagcgtcacctactgctctgtcagtcaggacttcccaggcagcaacttgaatttgctcaccaacaattctgggactcccaaagaccacgactcccctgagaacggtgcggacagcttcgagagctcagactccctcctccagtcctggaacagccagtcgtccttgctggatgtgcaacgggttccttccttcgagagcttcgaagatgactgcagccagtctctctgcctcaataagccaaccatgtctttcaaggattacatccaagagaggagtgacccagtggagcaaggcaaaccagttatacctgcagctgtgctggccggcttcacaggaagtggacctattcagctgtggcagtttctcctggagctgctatcagacaaatcctgccagtcattcatcagctggactggagacggatgggagtttaagctcgccgaccccgatgaggtggcccgccggtggggaaagaggaaaaataagcccaagatgaactacgagaagctgagccggggcttacgctactattacgacaagaacatcatccacaagacgtcggggaagcgctacgtgtaccgcttcgtgtgcgacctccagaacttgctggggttcacgcccgaggaactgcacgccatcctgggcgtccagcccgacacggaggactgaggtcgccgggaccaccctgagccggccccaggctcgtggactgagtgggaagcccatcctgaccagctgctccgaggacccaggaaaggcaggattgaaaatgtccaggaaagtggccaagaagcagtggccttattgcatcccaaaccacgcctcttgaccaggctgcctcccttgtggcagcaacggcacagctaattctactcacagtgcttttaagtgaaaatggtcgagaaagaggcaccaggaagccgtcctggcgcctggcagtccgtgggacgggatggttctggctgtttgagattctcaaaggagcgagcatgtcgtggacacacacagactatttttagattttcttttgccttttgcaaccaggaacagcaaatgcaaaaactctttgagagggtaggagggtgggaaggaaacaaccatgtcatttcagaagttagtttgtatatattattataatcttataattgttctcagaatcccttaacagttgtatttaacagaaattgtatattgtaatttaaaataattatataactgtatttgaaataagaattcagacatctgaggttttatttcatttttcaatagcacatatggaattttgcaaagatttaatctgccaagggccgactaagagaagttgtaaagtatgtattatttacatttaatagacttacagggataaggcctgtggggggtaatccctgctttttgtgtttttttgtttgtttgtttgtttgtttttggggggttttcttgccttggttgtctggcaaggactttgtacatttgggagtttttatgagaaacttaaatgttattatctgggcttatatctggcctctgctttctcctttaattgtaaagtaaaagctataaagcagtatttttcttgacaaatggcatatgttttccacttctttgcatgcgtttaagtcagtttatacacaaaatggattttattttttagtttaactgtgtttctccgacagctcacctctctctgaccacccagccatttccttcctgtgctccacgttcttctgtgtgattaaaataagaatattatttttggaaatatgcaactccttttcagagatcaggagggatttatgtagcagctatttttactgcaaaagtaattcactggaaaaaaaatgtaatttgtaagaaagctttatttttatctcagctctatgtaaagttaaagttactgtacagagctgaaggacggggggcggtaggggtcttgatgaaacctcttgaacgaagcacagtttgtcccatctttgttcactcgtgtgtctcaaccatcttaatagcatgctgctcctttttgctcagtgtccacagcaagatgacgtgattcttattttcttggacacagactattctgaggcacagagcggggacttaagatgggaaagagaaagcatcggagccattcattcggagaaaacgttttgatcaaaatggagacttttgtagtcgtttcaaaagagcacctgagtcatgtgtattcccggcctttataaatgacccggtcaagttggtttcaaagtccgacaggcttgtctgtttactagctgcgtggccttggacgggtggctgacatctgtaaagaatcctcctgtgatgaaactgaggaatcgggtggccgggcaagctgggaagagcaaagccagagctgcgctgcctcaatacccacaaaagaccattcccagtatacataagcacaggatgtttttctcaagagggatgtatttatcacttggacatctgtttataatataaacagacatgtgactgggaacatcttgctgccaaaagaatcctaggcagtggctcattgtatgtgaggttgaaccacgtgaaattgccaatattaggctggcttttatctacaaagaaggagtttcatggggttcagcctaacagttatggaaactacagtccttataaaccattggcatggtaataaacagatcttaagtataaaaattttgtaattgggcctttactctctcaataataaagtattttgtttatataaa

SEQ ID NO: 3 - RNA, 4060 nt, Homo sapiens
  source 1..4060: mol_type = mRNA
gccactcccgcggagcctgcgggatcggggcttcccgggagcagcgcgatcagcaccacgactcggggacacagccagggcccggtttctacaggaagcgcctcatttggagcctttttgtgatagaatgatcattagtcctaagcccattcagaggttcaagaatggggtcggctcaatttcagggccttattacccaagcccggctgcccttcggtgccaccagcaccactgctccgtcgctgcggaattccaaaggcaggtttggcgttagggccttggccccagagaggacgccgagcgctccacggaaagtctccgcccggctcccagggcgcacactcgcgcgcacgtggggccgaggccctgctcccggggcctcagggccagccggcgagggacccagccgagtgacagcaggaggcggagggaagctcagagctcccggagccgcccggccagcgtccggcctccctgatcgtctctggccggcgccctcgccctcgcccggcgcgcaccgagcagccgcgggcgccgagcagccaccgtcccgaccaagcgccggccctgcccgcagcggcaggatgaatgatttcggaatcaagaatatggaccaggtagcccctgtggctaacagttacagagggacactcaagcgccagccagcctttgacacctttgatgggtccctgtttgctgtttttccttctctaaatgaagagcaaacactgcaagaagtgccaacaggcttggattccatttctcatgactccgccaactgtgaattgcctttgttaaccccgtgcagcaaggctgtgatgagtcaagccttaaaagctaccttcagtggcttcaaaaaggaacagcggcgcctgggcattccaaagaacccctggctgtggagtgagcaacaggtatgccagtggcttctctgggccaccaatgagttcagtctggtgaacgtgaatctgcagaggttcggcatgaatggccagatgctgtgtaaccttggcaaggaacgctttctggagctggcacctgactttgtgggtgacattctctgggaacatctggagcaaatgatcaaagaaaaccaagaaaagacagaagatcaatatgaagaaaattcacacctcacctccgttcctcattggattaacagcaatacattaggttttggcacagagcaggcgccctatggaatgcagacacagaattaccccaaaggcggcctcctggacagcatgtgtccggcctccacacccagcgtactcagctctgagcaggagtttcagatgttccccaagtctcggctcagctccgtcagcgtcacctactgctctgtcagtcaggacttcccaggcagcaacttgaatttgctcaccaacaattctgggactcccaaagaccacgactcccctgagaacggtgcggacagcttcgagagctcagactccctcctccagtcctggaacagccagtcgtccttgctggatgtgcaacgggttccttccttcgagagcttcgaagatgactgcagccagtctctctgcctcaataagccaaccatgtctttcaaggattacatccaagagaggagtgacccagtggagcaaggcaaaccagttatacctgcagctgtgctggccggcttcacaggaagtggacctattcagctgtggcagtttctcctggagctgctatcagacaaatcctgccagtcattcatcagctggactggagacggatgggagtttaagctcgccgaccccgatgaggtggcccgccggtggggaaagaggaaaaataagcccaagatgaactacgagaagctgagccggggcttacgctactattacgacaagaacatcatccacaagacgtcggggaagcgctacgtgtaccgcttcgtgtgcgacctccagaacttgctggggttcacgcccgaggaactgcacgccatcctgggcgtccagcccgacacggaggactgaggtcgccgggaccaccctgagccggccccaggctcgtggactgagtgggaagcccatcctgaccagctgctccgaggacccaggaaaggcaggattgaaaatgtccaggaaagtggccaagaagcagtggccttattgcatcccaaaccacgcctcttgaccaggctgcctcccttgtggcagcaacggcacagctaattctactcacagtgcttttaagtgaaaatggtcgagaaagaggcaccaggaagccgtcctggcgcctggcagtccgtgggacgggatggttctggctgtttgagattctcaaaggagcgagcatgtcgtggacacacacagactatttttagattttcttttgccttttgcaaccaggaacagcaaatgcaaaaactctttgagagggtaggagggtgggaaggaaacaaccatgtcatttcagaagttagtttgtatatattattataatcttataattgttctcagaatcccttaacagttgtatttaacagaaattgtatattgtaatttaaaataattatataactgtatttgaaataagaattcagacatctgaggttttatttcatttttcaatagcacatatggaattttgcaaagatttaatctgccaagggccgactaagagaagttgtaaagtatgtattatttacatttaatagacttacagggataaggcctgtggggggtaatccctgctttttgtgtttttttgtttgtttgtttgtttgtttttggggggttttcttgccttggttgtctggcaaggactttgtacatttgggagtttttatgagaaacttaaatgttattatctgggcttatatctggcctctgctttctcctttaattgtaaagtaaaagctataaagcagtatttttcttgacaaatggcatatgttttccacttctttgcatgcgtttaagtcagtttatacacaaaatggattttattttttagtttaactgtgtttctccgacagctcacctctctctgaccacccagccatttccttcctgtgctccacgttcttctgtgtgattaaaataagaatattatttttggaaatatgcaactccttttcagagatcaggagggatttatgtagcagctatttttactgcaaaagtaattcactggaaaaaaaatgtaatttgtaagaaagctttatttttatctcagctctatgtaaagttaaagttactgtacagagctgaaggacggggggcggtaggggtcttgatgaaacctcttgaacgaagcacagtttgtcccatctttgttcactcgtgtgtctcaaccatcttaatagcatgctgctcctttttgctcagtgtccacagcaagatgacgtgattcttattttcttggacacagactattctgaggcacagagcggggacttaagatgggaaagagaaagcatcggagccattcattcggagaaaacgttttgatcaaaatggagacttttgtagtcgtttcaaaagagcacctgagtcatgtgtattcccggcctttataaatgacccggtcaagttggtttcaaagtccgacaggcttgtctgtttactagctgcgtggccttggacgggtggctgacatctgtaaagaatcctcctgtgatgaaactgaggaatcgggtggccgggcaagctgggaagagcaaagccagagctgcgctgcctcaatacccacaaaagaccattcccagtatacataagcacaggatgtttttctcaagagggatgtatttatcacttggacatctgtttataatataaacagacatgtgactgggaacatcttgctgccaaaagaatcctaggcagtggctcattgtatgtgaggttgaaccacgtgaaattgccaatattaggctggcttttatctacaaagaaggagtttcatggggttcagcctaacagttatggaaactacagtccttataaaccattggcatggtaataaacagatcttaagtataaaaattttgtaattgggcctttactctctcaataataaagtattttgtttatataaa

SEQ ID NO: 4 - protein, 469 aa, Homo sapiens
MNDFGIKNMDQVAPVANSYRGTLKRQPAFDTFDGSLFAVFPSLNEEQTLQEVPTGLDSISHDSANCELPLLTPCSKAVMSQALKATFSGFKKEQRRLGIPKNPWLWSEQQVCQWLLWATNEFSLVNVNLQRFGMNGQMLCNLGKERFLELAPDFVGDILWEHLEQMIKENQEKTEDQYEENSHLTSVPHWINSNTLGFGTEQAPYGMQTQNYPKGGLLDSMCPASTPSVLSSEQEFQMFPKSRLSSVSVTYCSVSQDFPGSNLNLLTNNSGTPKDHDSPENGADSFESSDSLLQSWNSQSSLLDVQRVPSFESFEDDCSQSLCLNKPTMSFKDYIQERSDPVEQGKPVIPAAVLAGFTGSGPIQLWQFLLELLSDKSCQSFISWTGDGWEFKLADPDEVARRWGKRKNKPKMNYEKLSRGLRYYYDKNIIHKTSGKRYVYRFVCDLQNLLGFTPEELHAILGVQPDTED

SEQ ID NO: 5 - DNA, 20973 bp, Homo sapiens
  source 1..20973: mol_type = genomic DNA; note = ETS2 genomic nucleic acid sequence, chr21:40176507-40197479
gtgattgtttcagccgtatagattctgactgtgactcatgccacggtgatacccggagaccttcggggacccagaaacattaaggtgcaccgatatctcaattatccggataactctagacctagagtcaccatggaaacctagaaccctgagtcacagagaagctgggccattcctctcccttcccggtggtgccgcagttcttgagatgtgagcagctttcagccccgtggcctcgcctaagctggggtttcctatttcatccacaacttcaggaatctaaactttcctgcaaaatattgagcatgtgacctgagtccaaacagccatggactcaagcaccttctgataaaaacggaaaccacagtgtagggggtggttttgaccagggtttcggagtcagataaactcaggtttccaccttgaatttctcatcaatctgacgtggcaggttattcatcctaaggttcagttcccacctgtgtaaagtgggagccgcaagtcctccgagagtgacgatgatgtgcgtggagtgcccagcccagattgaagcgcggccaaggcgggtcgctatctgggcaccgctcagctccagagggcgccactcccgcggagcctgcgggatcggggcttcccgggagcagcgcgatcagcaccacgactcggggacacagccagggcccggtttctacaggaagcgcctcatttggagcctttttgtgatagaatgatcattagtcctaagcccattcagaggttcaagaatggggtcggctcaatttcagggccttattacccaagcccggctgcccttcggtgccaccagcaccactgctccgtcgctgcggaattccaaaggcaggtttggcgttagggccttggccccagagaggacgccgagcgctccacggaaagtctccgcccggctcccagggcgcacactcgcgcgcacgtggggccgaggccctgctcccggggcctcagggccagccggcgagggacccagccgagtgacagcaggaggcggagggaaggttgggccggaaggtgtcagccccgccccgcgctccctcgccgcctccgccctcctcttctctcccctcgtgcgttccctctcctctccctccgctcccccaaccctcctgctgcccccttccctctcctcccgcttctccccatcctgcctacctcccttcccctcctcttctctctcctccccttccctcccctctctcttctctcctccctcgtttcctcccctcccctccactcggccgtccctccttcctcctccctcctccctcctcctcccgctcctgaagagcgcgccgcgtgggggacggcccggttacttcctccagagactgacgagtgcggtgtcgctccagctcagagctcccggagccgcccggccagcgtccggcctccctgatcgtctctggccggcgccctcgccctcgcccggcgcgcaccgagcagccgcgggcgccgagcagccaccgtcccgaccaagcgccggccctgcccgcagcggcagggtaagagctgggcccgcagagagcgcccggcgcgcggctccagtcccatggagggtcacccggggcctgggcgggggtcgcggggggcactgacacgcagatctcggggcgctgccgggggtgcaggtgggggtggcggctgctgcgaggactctaggggcgcgcgtctgagttccgcgccggctcgttttccggttatggagtggcctccggggctggcggggtcggccggggggttcctgcgtgctagggccgctgtcttcggggtcgcctagcggcgggcgcggccagggcgcgctggcttgtttcgctcgcttttgtttttaaaaggaaacgcaggcctggtagggggtcctgcccagtggatgtcccggcgaacatgatttcgcgaacgggagtgggggcacaggagagcgtgtccgaggtggcctggcgccccggctttgagggtgacttcctggagcggcgccgggcccggaggatctggggcgcccaagacacctgaaggctgcggcaccgcgggaacctgcggggcgcggggtgccatggtcacctgctcgccgcgtccagggcccgggctggggacccctcggtcgtgcgaggagagcgtggggaacctgtcggaaatgagatctggttgcgctgggctgcctttattttctcgttcctacagcatttgaatgaagaggtaactgagtgtttgcttgtgtgtgtttgggttgcgtgtgtgttatcctattttattttttacggcaggagaccttttatgttagcctgtacacaatttcagggtagcctaaaacagtagttgacgcgctagttatttagaaagtaaagaaatgagcagtcccctttggagacaggagtaatttattttaattcgccagtaagagattatatttgttccatacaatggaagcgcctgtgcgttatctgcctgcccacaccaccaccaatcagagagataaatctccacgttaagctttattaaaattctgagtagtagcgcagcacagtaaatgttagcaccgaccatccgcagggaaatgctcagatactcatttctgggattagtcattcatctctctaaactgatcgctttcaaagcgaaaaaagcaaactaccttcaaatgtgtgttacaaggttgtgttgtttcttttttttgtttgttttcaaatttatctaaataatcttacataagtgattacttgttccaataagggtactgttgtcaggcaaattctccttgtttttaaatgtaaccattctcagctgttacatttctgctctttatccttttttttttttttttttggtttaaaagcagcgatccatcagcaacaccaaacttgaaattgatttatgtggaaaaacttggcttgtctgccatctaacaagccctgttgagtaaaataagcaagcttaaatttgattagttgtgtgtctgcctgaaaccatttagtagagcactttaattctgcatggtttttacaaaactcattaaaagctggacaacaaaagaattctattctgtagctagtaattacagctctttatgtgggagtggtaggctgccttttctcctggtatttgtacacaaaagctggggagagcttttccactgccttccacattcattctctgtccatctcagagaagctgaaaaacagaatcaagtcagcaagcagtctgcaagcctaaggaagaaaaagagctgacccatccggctctggatggataactggcaacacccaagatgtctatgattttggccttctgtttgaggcttgtttatttctttaatcagcagctaccaggacttatttcatgcagagaaatgacataaagctctagaggaaggagcatttcacattggaaggagtagtgagaagggcctgatctgataatatcaggatgggtgggaagggtccattcctcatcattcatgttctcccggcacagaggactgtgtggaggactcaggatttaggaccatactcaactggggtttgatctccactttgccctgcggtggcccctctggtagctatgtggatcctgggcagattgtccatgttgctgggcctccgttgcttcactgtatgacgtctgcctctcagggatgcaggaagattcaattaggtaggagtggatggtcccccagcacggggcttagctcttagtaactaggcattctggcaatggcagccatcaataggagagactattgattgttgagtgaatataagtggcataatccaccaagtggagattctgagtgaatgtggccactttggacccaagactgctgcccctctctgtcctctcataggactattatttattgccagtgctgaccagcctttgttggctaccatgatcattacctgcactccgagtgttttaacagaattttctgatgcctctggcctcacctgctgccttttagccaagaggggtgtgtgtgtatgtgtgtgtgtgtgtgtgtgtgtgtgttgggtgtgtgttgggtagcagggttcttggaaggtctggtttagccataatgttgatcataaagagaaaacaagaaaataaatgcaaattaaatagttcggtgagcctttgttatttcgccacatccagtaagttctatcagttgaatgttgtctgaattacttaaaaagttctcagttgtaaacaggtaagagcacccagtggaggccatggagctaggcttagagctttgccaccagccttcagttccatagccatgaacctgggagggcaaggaatcaccctagaggtgaaggccattcatcggtgctaacccaaggtctctgaaaatgagttttagttactctattatctaagtatctttcttactgatatgtaacttacctggctgaacaattacattttagagctccaaataaattttcagcctattatgtcattgtccaattacaagcctcattgtaatagccatgttaaaaacaataaagccggtgaaattgatttcagtgtatttcaaattaacctaatatattcgaaatattatgtatgtataaaaattattgagcttttttcttttttccactaagtctttgagagctagtgtgtatgtgtggatttgtactagccactggttgagagctctgtagccacacatggcaagtgggtaccatattgcttagcccaggcctagtagaagattaagactcagtcagaggaagtaaggcagatccgtgcttggaatcagttcctcttgaagaagtgcacagccaagcaacacacaaagcagaaatattttgagctctgtattcaaagcaggaagatctgggaccatgtaagggggtttggtcttcagttctccctttgatcccttccctgggtgtgtgctttcttacagcctggagagtggaagggaagagagggggcttcctggactcctttcctgtctctagaaatgacccctgcagctgtcctgaagctctcaggttaagctgattgtgtccagagcagacaggaggcggcccactgccccactgtacccacccaagcactcacctaagctggccatggaaatgtgaatgaaaagcctgacccaggccctcttgccctgctaacccagcaaggaggctctgaccagccagccagcaagtgactttttctgtgacatctaagtggaattcagtgaggaatcagcattattattcaatcactgaaacagcatatcccattgtgatactgagaaatgctttcctcttgagacctttgggaggacagccatttaaacaagaattccctgcattcacttttgaattacctgttagctgtagaactcagccaggatcaagtcttgatgccaccaatgatgagtttagtgtacttaatcttttgcctctttgactttttttttttcttttttaagatgaatgatttcggaatcaagaatatggaccaggtagcccctgtggctaacagttacagagggacactcaaggtgagtgggcaagtcttaatttttttttttaattggaaaactcgatctctaggaggaaagaaaaaaaaggcctgggtcccagaaaactggttgtagccctagcttcttaatttattggccatgtgactatagactatttgcttacttgctctggtcctcagttttctggtctgcagaatgggtatcttgatgtgtgttgttcttctcctccacgatatgaaaccaaaggcctttatagcttgttttctgaggctccttccaggtgaaaatcctataactcccaataaaacagctggagaggctgtctctactgccttttctgtctacccaggagtttccttggttgatgggaagctgtttaagaactgaaatgagagagaagaggcagacagacccaaccgctacagctcctatcagaaagggcagcgcccgtggaattgtaggtagcctgttactggctcagagaacaagcaggcctacttatgttaaaccaaaaaccagatgaaaaatgaaagcgccctctctgctgcataaacattttatgtagcttctatcatcattccttttgggaacattctttagtagcacaaactggtaatctcccacttgtcttctccaaatatgacagcaagatttgattgttttcagtattatgtagtaaacatgtttcagaagcatgattttaaaattggcctcctcaaagtttagcgtcttgcataatgatgatgtacgtctctggcatattacattttcctttgtatatcattattgaggttatttgtctgatatgacccaaagaggcaaaactcagcacagtcctttctgcagtattctaaaggtcatcaaacttcagcctagtgagtctgcttgtttgatttggccggacattttaagcatggcagaagtggtacaagaaatcatggtattaagttgaaaccacaccccttagaaaaatccttctattaattcaaataatttgacgatgcttatgcggtttctgaaaagaagcagtcgttgctgaaattgatgtgttgaaataggaagcacagatttgtattgtcttttggcttcctggctttaaaaaaaaaaaaaaagaatttacggttaggaaaggcatttcttagtcacctgggcaatgcttgtgggaactttaaaagtttattggaggtttcaccaaaattagctctgctctaaagtagtttgtaaatccagatgagtaatttgcacttgcctgattgatcggttttcctcgtagatctcaggcagttggtcatcattgacttggatttttttgggctgggctccagcctctgcctgccctcttttgtaatgcgtcattcattgttctgcatttctaaaaagaaagcgggtaaccgattttatttgtaaatgtgttacacattttcttattgatgactcaacagatgggtcattgtattacacataacatgggatttccagcttatgttggaaaaatatagtctttcttcacgtgttcctcaacaatttaggcgatgatgaatccagatattatcggttactttcaaatgagtcttacattaaaaggtatttctttcataaatgttatgtaagatgctcagaaatcgagttagtgtaaggttttatctttctgtgtgtctcttcttttctgcagatagtttagtttaactttaaacctatttcctttggaaatctttccccttagtaggtgatttgggttttttttgttttgttttatttgttttttcgtttttggttttaagcaacagggtccttctctgttgcccaggcgggagtacagtggcttgatcgtagttcaccgcagacttgaactcctgggctcaagtgatcctctggcctcaacctcataaagcactggggttacaggcataagccaccccgcctggcctgggggcggggagggggttcatttttaagacactttacttgttataaaatgaaacgttaaggaattttgtcatgtattactattttccaaaccattggaattgatgtggaaaagcatctctagtttcagttaaattgtttcatggcaaacatttcaaaataaatgcttccaaagataaagtagtgtatttcattgtttgatttttcagtgtttatcgggtcatttgccttttaactgcattgtgctcagacggccttgtcaagggtcatttttcttacacacacagcctagttgaatttttctcaatgtaacaggagggtcaggtgtttggtaaaggggtctctgaggctcctctcgacaccattgtaagattctgttattttcagatttgtgtatttaatatattaacttaagtggattaggccactaaaacaaattcccagtcactccgactctgtgaggttacttaaataaatccacagtggccgggcaccgtggctcacgcctgtaatcccagcactttgggaggccaaggcgggtggattacacgaggtcaggagtttgagaccagcctggccaacatggtgaaaccctgtctgtactaaaaatgcaaaaattatccaggcgtggtggtgggcgcctgtagtcccagctactcgggaggctgaggcaggagaatcgcttgaacccaggagggggaggttgcagtgagccaagattgcaccactgcactctggcctgtgtgacagagtaagacctgtctcaaaataataataataataatccacagtaaaatacaatcatttcagtcactttgaccattttacgagtctgttttaaggactgactggttgattccatgtttgcttcaaaattgccagtttaatcagggactattggtaggattgcccacagaccacttttacaacaggtaattcaatcagtttaaatattgtatttccattttttttccatctgtttttgcagcgccagccagcctttgacacctttgatgggtccctgtttgctgtttttccttctctaaatgaagagcaaacactgcaagaagtgccaacaggcttggattccatttctcatggtaattggttcctcagacttgacaaattgtgcatgattttcctaagtagttcagttaataaagagatgacagttcctaagtgagaaatgttggtatctggaatctttgatatgaatagccagaagttctggacctgggagaaggcagtttttcatttgtatcaacttggtgattctgatgtggattctgatgtcttgagtgccagacgccccccagtgagccgctgttaggaaactgcaggggaacccctgagctgcccctcctggatgttttcaacattggcaagcaggtcctcatctccctgctctgccctggaaaacagacagaagagtagacattaaaacacacccaaacttgtaatttgggtttgcatcattagttaaatcatctcccctgccctgagttttggggcaacaaattgttctttgtaatccttacccatcggtagttctttctgtagttcttcgtgtcattgtttattcttcttaaaggaatagcacgtcagataatgttcatacaattactgtgacagagctattagttggaaacatgccagagatgactgtgtcatgtgaagtaggtgctgagaatacaggccagaaaattaattaaaacgcatacactggcgtaagcataaccctgcatggcagccatttatcattcttgaaattgctgagggtgtctggagaagattgtttttcaataaaaatggaaggtgtggttccattaccgtgctaagtgcggcacattcatatcacactcacttctcagctctacgcctagtttaatggaagtgacttccaaatgatacagtcccttcagccttttgaccagaaaccatgcaactgttttcattgcctacaattttaaaatagacttagcagttttccctacgcacaactgctttgtgacgctttgcaaaatagaatcaaatctgagttttattttagatttttttaataagtatagttctgagatatatattttctcaattatagtcagaaaagttttttgttaatagttacactgttttaaggaatcatgccaaggtttgagatcaaaattgttcttttccaaaaactaagatgtctctcctaaatctccacctgatatcaccaacttgaagtcctaatgtccccatggggggtttccttccagactccgccaactgtgaattgcctttgttaaccccgtgcagcaaggctgtgatgagtcaagccttaaaagctaccttcagtggcttcaaaaaggaacagcggcgcctgggcattccaaagagtaagtactgctttcctgagcctgcactgggtgagaagaaccaacttcagtgcagttgtttgatcttgacttgtttattaagcttttgcttggggtattctgcaaagagtagcatggatgtcgttaacctgagccagtttctgtttcaccccaatcaacccaagacttgatccagaactcattaaatatgtagtagaaggacgcattactggtgtcttgaaatgtgcctgggtcgtgtcagaatggtgacgtgtcatcatggtatcttgctcattcgtgggttctggtgtatgtcggtacttggtgcataaattagggatgacagtggtctcactacctttccactgtttttacctcatgtgttccattttttcttctctcctggtagacccctggctgtggagtgagcaacaggtatgccagtggcttctctgggccaccaatgagttcagtctggtgaacgtgaatctgcagaggttcggcatgaatggccagatgctgtgtaaccttggcaaggaacgctttctggagctggcacctgactttgtgggtgacattctctgggaacatctggagcaaatgatcaaaggtaccagctgaacgtcttacttctccttgtccaggatgagctgtggccgggaagactgattgggaaagtcacgtgggtgttcttcaaccttagggttgccacttgaaatgacatagagtaccttgcctcaaaatgccactcaagtgagtcagatatatggcagtgattatagatttttatcccactttatgtgagtgtgtgtgtgtgtgtgtgtgtgtgtgtatagcatcaagtatagccacaaggtagtagccttagtcactaaattgtttgctattgctggctgtattcatcacgggagtccatgttgactcagatatgtaggacagcaagaattcccacgtcttctgagctcaccttacagagctaagagatatggcagccttaatgaaaggggggagcctttactcacataaaccctgtgaattctactgtaaattttcctacatccaaatggaatgtattttggatgttgaggattgttgagggggcggggtctgtttttaaacaagtgagaaattgatatatatttacacgctatcatattgtacagaaactggccattgatcagtagaaatctcaccaaatacagagaaagtcttgattcaaaacttaaaatagcagggagaccaatgtttaaaacatgttgagttaaaaaagaaaagagggccaggtgcggtggctcacacctgtaatcccagcactttgggaggcggaggcgggcagattacttgaggttaggagttcaagaccagcctagccaatatggtgaaaccctgtctctactaaaaatacaaaaaataaaaatattagctgggcatagtggcacatgcctgtaatcccagatactcggaagcctgaggcaggagaatcccttgagcccgggaggcagaggttgcagtgagtcgagatcacaccactgcactccagcctgtgggagtgagactctgttgaaaggaaggaaggaaggaaggaaggaaggaaggaaggaaggaaggaaggaaggaaggaaggaaggaaggaaggaaggagggagggagggaaggaaggaaggaaagaaagagggagggagggaggggagggagggagggagggagggaggggagggagggagggagggagggagggaaagaaataaaatacagtagggattatttaggttaaaaaaatataagaaaataagcatgtttatagaattcagggaactagtcctcacctgactgattttaaacacactgaatttcagcatcaactgtagactcagaatctcgaaaacacctaggattcaacagctaactttttttagttacggcgtctgagggccagggaaactcatttttaaagaccctcatttcttctgaacttggacagggcttttctaccaagtttctggtgatctgtttattggcaaattgggcctgttttaccattcctcgggcctgtgagtttgtgtgtatttgggtttccgggtggacgtgcactttgtaatatattcgtagtaagtatgcatacacagtgcacaaaccttttccctttcaaagttcttgggcacatgtgttccccatcccagactcccacagccctggagacttcccacaccctggggaagcgtgcaggtttggaagcagtggaactgtaagcttcacgggggcagacaccatccattttattaatttcctgtatgcagcctctggtacagatggcatttcataaatacttaactgtttcttgaatgcatgaatgaattcaatacaccatctggaccttttgcgatagaaaatttagtgttgccaatgacatcattcattttggaaaatttattttagtaattggaaaaatgggttgctttctctcgttttttgaaaaagacaggcagggacgtggagcaccatgagattaaatgtgaagcactccgtgcatctcatcattctaaaattatcatctcacaggaattcagaaagtcaatttgttctgtgtagttttcacctacatctgccatcttacgtctcctgtgtctgcttttcagaaaaccaagaaaagacagaagatcaatatgaagaaaattcacacctcacctccgttcctcattggattaacagcaatacattaggtcagtccgattgattctgcccttaagaactttgtcttcagtcttcccagtagacttggaatctctctactgtaggctcctaaggggccacctagacctgtgtgtctcttatggtggccacacgtgggtgttgagctataagccattgaaatgtgactggtccacatcgagatgtgtaggaagagtatagtcatgcacaccagatttggaagtcttagtatgaaacagagaatataaaatgtctccctgataatttttaatgtattgattacatattgaaatgataattgctatatgaggccaagtaaaataagaaaattaacttcacctgtttctttttgcttttttaatatggccactagaactttcgcagttgcttaagtggctcacacgatcttccataggtcagcactgttagtgctagtccaggcaattcttagagcaaggagcagaacgaagtgagctgattaccgcagtcaccagtaatgttggtttagtgtagcaaacaagaggcggcaggcacagagtgcgtgaaaagcaatttgtaaactggctctgaacttgggagcttaaagagctgtcatggaatctgggtcctttcttctggttgctgatactggggagggcacagggtctggccaggccagcgagggaggggtcctcggaacagcaggcttgggtggtgttggtccggcccagtgttttctctctggccccagctccaccattcagacccggtgaagcccacctctctgccccactgggggcttcctgctcaggaggtctttctgaacatgacagtgggacaccctgtgcctgcctgaacctgaatcagagtcagagagcttccttccctctccagaaaataaattattctgcaatgaacagagacaggagcggtgtgatgaaggagcagaaagcagagtgggctgtgcctcagcacacagggttcatggcctggttctgcccttatgaatcacacagtacatgacacgtcacttggtctgtctgagccttactctcctcggaagctaaaagagccagtgagccctgtcttcctgaaggtcccatgaaagcactagggttgtagaaataggaacattgagtcctgcctgcagaggctgcagtcattttggcagagggctgctggttgagaagctgaatggtttgagaagaaggcctggtgacagacccccaccaaagcccgggtctccactgaggttctgcagagggctggagtgtgcggagtgctcacctgtcatttgctcgtaggggttagttactggggtaacactgactttaagagctctgccgtccgattgttctgttccaggttttggcacagagcaggcgccctatggaatgcagacacagaattaccccaaaggcggcctcctggacagcatgtgtccggcctccacacccagcgtactcagctctgagcaggagtttcagatgttccccaagtctcggctcagctccgtcagcgtcacctactgctctgtcagtcaggacttcccaggcagcaacttgaatttgctcaccaacaattctggtaagattggaagcatctttcaacaaggctgttgctttgattctgagaaccccagagccataatgaacctcttaataaatacttcctggattcagccattagagaagggggtcaaagcccatgttctaagtggatttccaacaagcatacccctaatatgtttcaggcactggcccaggtactgcaggcattgagtaaacaaaacagtccctgctcttacagccagatttctccacactttgggatatttatggaggcgatgcaggaatgtgacatttcttcttatggagatgtaacataaatacagtgcacaagtctcaggtcatgtggctggttggatttgtgtatgtatgtatacccatgtaataaccatccagatggagatagagaagtttccagtagcctggaagattccttcgtaccccttcccatcaataatgtccccccgacagaagggaacgattctgacttttatcattacggattaggttttgattttcttgaactttacatagatggaatcataaggcatgttttcttttgtgtttgtcttttactcatcatgtctgggattcatgcatgttgttggatgtagcagccatgtgttctttttcatcgctgtatagtattccattataagaattttattcattctactgttgatggaagataggatttgtgtagtatagttaccctaaggaataagcacctgagtagagagcattttgagaagtctgagtggttctacaccagcaggtgcatgattgcactggtagtggttggtgatgctatggtcgtgcccaagaccaactgtgtcctggaggaatcaaagtatgtgtttggttgtctttgccagggactcccaaagaccacgactcccctgagaacggtgcggacagcttcgagagctcagactccctcctccagtcctggaacagccagtcgtccttgctggatgtgcaacgggttccttccttcgagagcttcgaagatgactgcagccagtctctctgcctcaataagccaaccatgtctttcaaggattacatccaagagaggagtgacccagtggagcaaggcaaaccagttatacctgcagctgtgctggccggcttcacaggtgtgtgtggaactccgagagcctggccgcccagtctcctgggtcctgtcccttgcttcctttcgagccacagtaccacattcaccgagggtgtttctaagctaggtacaccagtgctctacactccatgttttatgcgtggcttgctgtatctctgaattcgacaaaagccacacttggaggaattttcatgtacacagagctccatggaatgagtgaaaattgacaggccactccctccacctgggacgttctgaggaatagttggagcaggggacatggcctgtgcagtcttcagccctgactcacccactccaagaatctcaaagaatgtaaactggcttagggaggggcagcagagccagtctcaaataatatgggcactgtttaccttttcttgcttctaaaatctatctgctcttatttttgaggtttgggtgagagctattctaggcagtggacaaatataaagctgtttttagaaacaggtatttccagtactttagcgaaaatactggggctttgttttcactgcttcgtgtatccttaaatgcgtttttgtttttgtttttttaaccatatggagccactgtggattgaaattacctaacttaaaaaaatctgataagtttaaaataaggatactgaggcattcattctttttctgtcatctaagaacaatcagccacgtaaacgtattaaaatacacggaaagagtgtgtgaaagccattatcggtttcattattgggagtttagggcctgagaagaataaacagccataacgttagtgtggttcattttaatagtaacataagtttctgtttattgactgccaactctgtagttaagtaggcatacagtctgtaggacagcctggcatgggggtggtatgaggccattttacagatggggaaattggctcacaggcagacagctatcggtgctcacacagctctggttctagaatgcctgtcccgtgctttacagcaaagctttattgataacggccattgcgtttctgaggaagagctatttggtagaaagaggttgggcttagggtcggaagatctaggctcttctcctgtttgctgtgatgggcgtgtgatccaggacaaggcacctggcctctgagccgcatcttatccctcctctttagaacagaaccacactgacacctgctttgcaaggtggtagttctaaatgtcaatcgaggacaggtggacgagaagctcttggcaaagcatgcttcaccagacagaggctgtgaggcaacgggtgtgaccccgcgtggctattgagtaaaccgggctcacattaagcaatttacacatggtcatttggcaggaaggtggcatgggatcccacgtatgacacgcctcattctgtgatgaaacaccccctcagccacccccatctctgccccactgggtgtttctagaaaaaaaccttgaaggtttttacataaggaacaattaaaccagatggggaattttaatgccaagagttggtggccctaaactttcttccagaaagaacaaacctagcaatcaaggggaagagtgtctcgcctagtgaccttattttctaggagtaggcagtgtggagcaggaactcacatttggtgccccgccccatcccgttaaagcacttagtactgtcaccaacaccttgagtgccaccctgagtgtaacatcggaaccccattcagagagttgggtctgcattcctaaatcagcatgtacaattaggatggttaaagacttgctgtattttacatctgtgaaagggtatgatccgtctccctccctctccccgcaggaagtggacctattcagctgtggcagtttctcctggagctgctatcagacaaatcctgccagtcattcatcagctggactggagacggatgggagtttaagctcgccgaccccgatgaggtatggccagagccctgggaaatctctgggcttgaaaacctgatttcctgcttgcattcaaaaactcagttctttgggcacaaaaaagggttcaccagtactgctgagaatctttccacgtgaggcatccttggctgttgggaaaatggaagtggagtcattgctttgttgataaacgtgtacagtgttttctgggtatgcttcatacaagggcgttgcacagatttcactgtcttgatcagttgtctgatcaagaggcccaagctgcaaactgaggttctctgctgaccatctgaaaatgccttaggaaggctgcccttgtccatcgggggaacaagcctcatgtggccccgagccagccttctgtccactccttagctgagcccagcagtgttgaccccagccctgcagagttagaaccaggagccctgtctaaaggagaaggccaagggcaggtgggataccaaccccgccagaaaagcgttagagagaagcccttggtctctggcactggggagcgtaatctgtcaccattcccaccccgcctccagttcccttgggagacaagcattgaggaatgagtatatcacacagctcagaatcccactcggcacctgttaacttcagtagttggaatatccagcctggagggtgggtgacaccacctttccctggtctcaggaccctcctggctctgaacccttggctcccaggaggtttcactgagctggggccagggagcagggacctcattccccagtggttctgccccttggggacacagtgcccctaccataggtactcaaaggtactcagaggtactcaaaaggtcctcctgcggaccttgtgtaggtgtcaagttctctctagagtgaacatgcctcagaatcataatcagggaggaatgtcattcactttttcttcattgacaaattgagtttaactcttttccatccatgttcaccaaaggtggcccgccggtggggaaagaggaaaaataagcccaagatgaactacgagaagctgagccggggcttacgctactattacgacaagaacatcatccacaagacgtcggggaagcgctacgtgtaccgcttcgtgtgcgacctccagaacttgctggggttcacgcccgaggaactgcacgccatcctgggcgtccagcccgacacggaggactgaggtcgccgggaccaccctgagccggccccaggctcgtggactgagtgggaagcccatcctgaccagctgctccgaggacccaggaaaggcaggattgaaaatgtccaggaaagtggccaagaagcagtggccttattgcatcccaaaccacgcctcttgaccaggctgcctcccttgtggcagcaacggcacagctaattctactcacagtgcttttaagtgaaaatggtcgagaaagaggcaccaggaagccgtcctggcgcctggcagtccgtgggacgggatggttctggctgtttgagattctcaaaggagcgagcatgtcgtggacacacacagactatttttagattttcttttgccttttgcaaccaggaacagcaaatgcaaaaactctttgagagggtaggagggtgggaaggaaacaaccatgtcatttcagaagttagtttgtatatattattataatcttataattgttctcagaatcccttaacagttgtatttaacagaaattgtatattgtaatttaaaataattatataactgtatttgaaataagaattcagacatctgaggttttatttcatttttcaatagcacatatggaattttgcaaagatttaatctgccaagggccgactaagagaagttgtaaagtatgtattatttacatttaatagacttacagggataaggcctgtggggggtaatccctgctttttgtgtttttttgtttgtttgtttgtttgtttttggggggttttcttgccttggttgtctggcaaggactttgtacatttgggagtttttatgagaaacttaaatgttattatctgggcttatatctggcctctgctttctcctttaattgtaaagtaaaagctataaagcagtatttttcttgacaaatggcatatgttttccacttctttgcatgcgtttaagtcagtttatacacaaaatggattttattttttagtttaactgtgtttctccgacagctcacctctctctgaccacccagccatttccttcctgtgctccacgttcttctgtgtgattaaaataagaatattatttttggaaatatgcaactccttttcagagatcaggagggatttatgtagcagctatttttactgcaaaagtaattcactggaaaaaaaatgtaatttgtaagaaagctttatttttatctcagctctatgtaaagttaaagttactgtacagagctgaaggacggggggcggtaggggtcttgatgaaacctcttgaacgaagcacagtttgtcccatctttgttcactcgtgtgtctcaaccatcttaatagcatgctgctcctttttgctcagtgtccacagcaagatgacgtgattcttattttcttggacacagactattctgaggcacagagcggggacttaagatgggaaagagaaagcatcggagccattcattcggagaaaacgttttgatcaaaatggagacttttgtagtcgtttcaaaagagcacctgagtcatgtgtattcccggcctttataaatgacccggtcaagttggtttcaaagtccgacaggcttgtctgtttactagctgcgtggccttggacgggtggctgacatctgtaaagaatcctcctgtgatgaaactgaggaatcgggtggccgggcaagctgggaagagcaaagccagagctgcgctgcctcaatacccacaaaagaccattcccagtatacataagcacaggatgtttttctcaagagggatgtatttatcacttggacatctgtttataatataaacagacatgtgactgggaacatcttgctgccaaaagaatcctaggcagtggctcattgtatgtgaggttgaaccacgtgaaattgccaatattaggctggcttttatctacaaagaaggagtttcatggggttcagcctaacagttatggaaactacagtccttataaaccattggcatggtaataaacagatcttaagtataaaaattttgtaattgggcctttactctctcaataataaagtattttgtttatataaattctttgtgatagtcctcgttcttcctctccacacccagcatgaaggagttggaggaaggatgttaaccccagatccattctctactcaaaacattccatcatcaagcggcaagtctctgtttaactggtttatacacaagtcacttagaaaccacaacccaaattggaatcaactttgagcccttcctaaaagaattcccaaaaagtgctctctttcaaaacaaaaaattcttttaagaaagtgttataatagaaagattcaaatgtctttctttgccagaagcttggcagagataacagaggagagattctggaatgtttatattaagtacattagaatttgaggtttaagtacttttggaactgaggccacaacactctgtcccctcagtggagtctgacattggtaaggtgatggtggtcttaggacacctgttttcaaatttgttctctttacatgctccaaaattagtgagtatgccaaaaagcttatattcattggtgcgctagagccagctcactgtgttctcccgagagctacatgtcccttcccatcatcgcatcagtaatggtagcttgagatggggcgtgatgagagt

SEQ ID NO: 6 - RNA, 20 nt, synthetic construct
  source 1..20: mol_type = other RNA; note = chr21q22 5 gRNA
cctggctgcctcgcgtttcc

SEQ ID NO: 7 - RNA, 20 nt, synthetic construct
  source 1..20: mol_type = other RNA; note = chr21q22 3 gRNA
cctcgtccaacagagagcaa

SEQ ID NO: 8 - RNA, 20 nt, synthetic construct
  source 1..20: mol_type = other RNA; note = ETS2 gRNA1
cagacacagaattaccccaa

SEQ ID NO: 9 - RNA, 20 nt, synthetic construct
  source 1..20: mol_type = other RNA; note = ETS2 gRNA2
ttgctgcacggggttaacaa

SEQ ID NO: 10 - DNA, 20 bp, synthetic construct
  source 1..20: mol_type = other DNA; note = chr21q22_Fw primer
ggtggggagagttccaaagg

SEQ ID NO: 11 - DNA, 20 bp, synthetic construct
  source 1..20: mol_type = other DNA; note = chr21q22_Rv primer
tcacccttcacctctttgct

SEQ ID NO: 12 - DNA, 20 bp, synthetic construct
  source 1..20: mol_type = other DNA; note = ETS2_g1_Fw
tcctgaaggtcccatgaaag

SEQ ID NO: 13 - DNA, 20 bp, synthetic construct
  source 1..20: mol_type = other DNA; note = ETS2_g1_Rv
tcattatggctctggggttc

SEQ ID NO: 14 - DNA, 20 bp, synthetic construct
  source 1..20: mol_type = other DNA; note = ETS2_g2_Fw
gcggcacattcatatcacac

SEQ ID NO: 15 - DNA, 20 bp, synthetic construct
  source 1..20: mol_type = other DNA; note = ETS2_g2_Rv
gcagaataccccaagcaaaa

SEQ ID NO: 16 - DNA, 16 bp, synthetic construct
  source 1..16: mol_type = other DNA; note = 16-nt universal primer site
actggccgcttcactg

SEQ ID NO: 17 - DNA, 12 bp, synthetic construct
  source 1..12: mol_type = other DNA; note = KpnI and XbaI restriction sites
tggacctctaga

SEQ ID NO: 18 - DNA, 17 bp, synthetic construct
  source 1..17: mol_type = other DNA; note = 17 nt universal primer site
agatcggaagagcgtcg

SEQ ID NO: 19 - DNA, 50 bp, synthetic construct
  source 1..50: mol_type = other DNA; note = ETS2 Fw primer
gctaatacgactcactataaggacaggccaccatgaatgatttcggaatc

SEQ ID NO: 20 - DNA, 24 bp, synthetic construct
  source 1..24: mol_type = other DNA; note = ETS2 Rv primer
gccaccatgaatgatttcggaatc

SEQ ID NO: 21 - DNA, 50 bp, synthetic construct
  source 1..50: mol_type = other DNA; note = GFP Fw primer
gctaatacgactcactataaggacaggccaccatggtgagcaagggcgag

SEQ ID NO: 22 - DNA, 23 bp, synthetic construct
  source 1..23: mol_type = other DNA; note = GFP Rv primer
ttacttgtacagctcgtccatgc